SEQUENCE LISTING (26 sequences)

SEQ ID NO: 1 - DNA, 19 bp, Artificial sequence
  Primer
acctccgattccgagcttt

SEQ ID NO: 2 - DNA, 19 bp, Artificial sequence
  Primer
acctccgattccgagcttt

SEQ ID NO: 3 - DNA, 20 bp, Artificial sequence
  Primer
cagagcagcagtacccccta

SEQ ID NO: 4 - DNA, 20 bp, Artificial sequence
  Primer
cctctcggccatacctgata

SEQ ID NO: 5 - DNA, 20 bp, Artificial sequence
  Primer
tgacgatgaggaagtcgatg

SEQ ID NO: 6 - DNA, 20 bp, Artificial sequence
  Primer
cttctccgacccaagagatg

SEQ ID NO: 7 - DNA, 20 bp, Artificial sequence
  Primer
cgagcgagccagagatagag

SEQ ID NO: 8 - DNA, 20 bp, Artificial sequence
  Primer
gggctcaccaggtattcaga

SEQ ID NO: 9 - DNA, 20 bp, Artificial sequence
  Primer
acgaattgcctgtgaggaag

SEQ ID NO: 10 - DNA, 22 bp, Artificial sequence
  Primer
tgggtctttctctttttcatcc

SEQ ID NO: 11 - DNA, 20 bp, Artificial sequence
  Primer
gacctgctcccttttcccta

SEQ ID NO: 12 - DNA, 21 bp, Artificial sequence
  Primer
agccagtctgcaaatatcacc

SEQ ID NO: 13 - DNA, 19 bp, Artificial sequence
  Primer
agcgggaagaggatggatt

SEQ ID NO: 14 - DNA, 20 bp, Artificial sequence
  Primer
cccagggaggtacctgaagt

SEQ ID NO: 15 - DNA, 20 bp, Artificial sequence
  Primer
tgaaaagggtcctggagaaa

SEQ ID NO: 16 - DNA, 20 bp, Artificial sequence
  Primer
cgtttgatcaccatgacctg

SEQ ID NO: 17 - DNA, 20 bp, Artificial sequence
  Primer
accactcagagtccccaaag

SEQ ID NO: 18 - DNA, 20 bp, Artificial sequence
  Primer
tctgggcatcttctcctctt

SEQ ID NO: 19 - DNA, 20 bp, Artificial sequence
  Primer
ccatttttaggccgaggagt

SEQ ID NO: 20 - DNA, 20 bp, Artificial sequence
  Primer
atggctacaactccgacagg

SEQ ID NO: 21 - DNA, 20 bp, Artificial sequence
  Primer
cccagagaagttcgagaagc

SEQ ID NO: 22 - DNA, 20 bp, Artificial sequence
  Primer
gccatgtagcactggttctg

SEQ ID NO: 23 - DNA, 20 bp, Artificial sequence
  Primer
gcttgcttgccttttgtttt

SEQ ID NO: 24 - DNA, 21 bp, Artificial sequence
  Primer
tctcaatcaccgtcctctgtt

SEQ ID NO: 25 - DNA, 21 bp, Homo sapiens
ctgggttgtttcaatctaaca

SEQ ID NO: 26 - DNA, 21 bp, Homo sapiens
acggtcaataagaccagataa

(19) United States
(12) Patent Application Publication
(10) Pub. No.: US 2009/0275633 A1
(43) Pub. Date: Nov. 5, 2009

(54) Novel Tumour Suppressor

(75) Inventor: Manel Esteller, Madrid (ES)

Correspondence Address: FOLEY AND LARDNER LLP;SUITE 500, 3000 K STREET NW, WASHINGTON, DC, 20007, US

(73) Assignee: ONCOMETHYLOME SCIENCES SA

(21) Appl. No.: 12/225,807

(22) PCT Filed: Apr. 13, 2007
(86) PCT No.: PCT/GB2007/001347

§ 371 Date: Dec. 12, 2008

Related U.S. Application Data
(60) Provisional application No. 60/744,800, filed on Apr. 13, 2006.

(30) Foreign Application Priority Data
Apr. 13, 2006 (EP) 06112667.8

(51) Int. Cl. A61K 48/00; C12Q 1/68; C12Q 1/34

(52) U.S. Cl. 514/44 A; 435/6; 435/18

(57) ABSTRACT

A method of diagnosing cancer includes the steps of, in a sample obtained from a subject, determining the level or activity of HDAC2. A reduced level or activity of HDAC2 is indicative of cancer. HDAC2 protein expression is preferably determined. Indirect determination of HDAC2 expression is also possible, preferably by looking at the expression of genes selected from NCOA4, CTSB, TBCD, PPP2R4 and CORO1C. Methods for predicting the probability of successful treatment of cancer using a hydroxamic acid based HDAC inhibitor and for selecting suitable cancer treatment regimens can also be based upon determining the level or activity of HDAC2. Methods of treatment of cancer may be based upon use of carboxylic acid based HDAC inhibitors or by reconstituting HDAC2 activity.

### TECHNICAL FIELD

The present invention relates to methods and products useful for diagnosing and treating cancer and is based around the unexpected finding of HDAC2 mutations which are associated with cancer.

### INTRODUCTION

Widespread changes in DNA methylation (1,2) and post-translational modifications of histones occur in cancer cells (3,4) and both marks have a crucial role in chromatin packaging and gene expression (1,2,5,6). We are largely ignorant of the mechanisms underlying the disruption of the epigenetic landscape in transformed cells.

Histone Deacetylases (HDACs) are well known targets for treating cancer. The rationale behind attempting to inhibit HDAC activity is that in many cancers expression of tumour suppressor genes may be down regulated due to the action of HDACs, such as HDAC2. HDACs cause deacetylation of histones located in the promoter regions of these genes. A range of HDAC inhibitors (HDACis) are in clinical trials (13) for treatment of various cancers. Thus, for example, in colorectal cancer, HDACis are capable of inhibiting tumour growth in cell lines (13, 14) and in APC (min) mice (15). HDACis include hydroxamic acids, such as trichostatin A and also carboxylic acids such as butyrate and valproate.

### DESCRIPTION OF THE INVENTION

The present invention is based around the surprising discovery that HDAC2 itself actually appears to fulfil a tumour suppressor role. A mutation in HDAC2 which leads to truncation of the protein and loss of HDAC2 function has been found to be associated with cancers, in particular those cancers displaying microsatellite instability. Recovery of HDAC2 function has also been shown to induce tumour-suppressor like features in these cells. Thus, in complete contrast to previous perceptions, loss of HDAC2 function may actually be indicative of a transformed cell. Furthermore, functional abrogation of HDAC2 in cancer cells also provides the cells with an altered sensitivity to certain cancer treatments. These discoveries have a number of applications which are expounded below in further detail.

### Diagnostic Methods of the Invention

The loss of HDAC2 function is an indicator of cancer. Accordingly, in a first aspect, the invention provides a method of diagnosing cancer comprising, in a sample obtained from a subject, determining the level or activity of HDAC2, wherein a reduced level or activity of HDAC2 is indicative of cancer. Preferably, a substantially total loss of protein expression or activity is determined. This is particularly relevant in the case of colon cancers. Partial loss of activity has also been shown to be relevant to cancer. In particular heterozygous mutations in the HDAC2 gene have been shown for the first time herein to be linked to the incidence of cancer. In a particular embodiment, the cancer linked to a heterozygous HDAC2 mutation comprises endometrial cancer.

Note that the name “HDAC2” is the standard nomenclature approved by the human genome organisation for this HDAC and its encoding gene, to ensure that each symbol is unique. The listed accession number for this gene is U31814 and the chromosomal location is 6q21. Further details can be found at www.gene.ucl.ac.uk/nomenclature.

In one preferred embodiment, the cancer which is being diagnosed is one which displays microsatellite instability (MSI). According to the present invention, as detailed in the experimental section below, a frameshift mutation in HDAC2 in cancer cell lines with MSI leads to a loss of HDAC2 expression. Thus, the loss of HDAC2 may be used as an indicator of this particular cancer type.

The method of this aspect of the invention may be utilised to diagnose cancer in general. In one particular embodiment, the method is utilised to diagnose any of colorectal, gastric and/or endometrial cancer. In one preferred embodiment, the method is used to diagnose hereditary nonpolyposis colon cancer and/or sporadic colorectal cancer. All of these cancer types may be MSI associated.

“Diagnosis” is defined herein to include monitoring the state and progression of the disease, checking for recurrence of disease following treatment and monitoring the success of a particular treatment. The tests may also have prognostic value, and this is included within the definition of the term “diagnosis”. The prognostic value of the tests may be used as a marker of potential susceptibility to cancer. Thus patients at risk may be identified before the disease has a chance to manifest itself in terms of symptoms identifiable in the patient.

The nature of the mutation which causes a decrease in the level or activity of HDAC2 is not limiting with respect to the invention. The most important aspect is that a loss of HDAC2 function has been shown for the first time herein to be linked to the incidence of cancer and also to the effectiveness of certain anti-cancer agents for treating these cancers. Thus, any type of mutation leading to functional abrogation of HDAC2 is included within the scope of the invention. In one preferred embodiment, the mutation occurs in a microsatellite repeat. In particular, the mutation may occur in the (A)9 microsatellite. In a further embodiment, the mutation occurs in a coding exon. In another embodiment, the mutation is a frameshift mutation, particularly a truncating mutation. Single nucleotide polymorphisms which lead to a reduction in the level or activity of HDAC2 may also be included within the scope of the invention. Mutations which cause deletion, substitution or addition of one or more amino acids to HDAC2 as compared to the wild type sequence are also included within the scope of the invention, as are point mutations, inversions and translocations, with the proviso that the mutation must be one which functionally abrogates HDAC2, thus contributing to, or representing an indicator of, cancer.

The method according to the first aspect of the invention is most preferably an ex vivo or in vitro method carried out on an isolated sample. In one embodiment the method may also include the step of obtaining the sample.

The test sample is most preferably a tissue sample, taken from the subject, which is suspected of being tumorigenic. In a preferred embodiment, the sample comprises a colon, rectal, endometrial or stomach sample. However, any other suitable test sample in which activity or levels of HDAC2 can be measured to indicate the presence of cancer are included within the scope of the invention. Test samples for diagnostic, prognostic, or personalized medicine uses can be obtained from surgical samples, such as biopsies or fine needle aspirates, from paraffin embedded tissues, or from a body fluid.

The decreased level of expression or activity of HDAC2 may, as necessary, be measured in order to determine if it is statistically significant in the sample. This helps to provide a reliable test for diagnosing cancer, in particular MSI cancers. Any method for determining whether the expression level or activity of HDAC2 is significantly reduced may be utilised. Such methods are well known in the art and routinely employed. For example, statistical analyses may be performed using an analysis of variance test. Typical P values for use in such a method would be P values of <0.05 or 0.01 or 0.001 when determining whether the relative expression or activity is statistically significant. A change in expression or activity may be deemed significant if there is at least a 100 decrease for example. The test may be made more selective by making the change at least 15%, 20%, 25%, 30%, 35%, 40% or 50%, for example, in order to be considered statistically significant.

In a preferred embodiment, the decreased level of expression or activity of HDAC2 is determined with reference to a control sample. This control sample is preferably taken from normal (i.e. non tumorigenic) tissue in the subject, where HDAC2 expression and activity is present. Additionally or alternatively control samples may also be utilised in which there is known to be a lack of HDAC2 activity and expression.

Suitable additional controls may also be included to ensure that the test is working properly, such as measuring levels of expression or activity of a suitable reference gene in both test and control samples.

In a most preferred embodiment, the subject is a human subject. Generally, the subject will be a patient wherein cancer is suspected or a potential cancer has been identified and the method may be used to determine if indeed there is a cancer present. The methods of the invention may be used in conjunction with known methods for detecting cancer.

By “level” is meant the level of expression of HDAC2. Such measurements may preferably be carried out at the protein level, but may also be carried out at the RNA level. Changes in the level of expression may be measured directly or indirectly. Indirect measurement may involve determining expression of genes whose expression is modified or at least partially determined by HDAC2 activity.

In one preferred embodiment, the diagnostic method of the invention is carried out by determining HDAC2 protein expression. In a most preferred embodiment, total loss of wild type HDAC2 protein expression is observed in the sample in order to conclude a diagnosis of cancer. However, partial loss of HDAC2 expression may also be relevant.

Levels of protein expression may be determined by a number of techniques, as are well known to one of skill in the art. Examples include western blots, immunohistochemical staining and immunolocalization, immunofluorescene, enzyme-linked immunosorbent assay (ELISA), immunoprecipitation assays, agglutination reactions, radioimmunoassay, flow cytometry and equilibrium dialysis. These methods generally depend upon a reagent specific for identification of HDAC2. The reagent is preferably an antibody and may comprise monoclonal or polyclonal antibodies. Fragments and derivatized antibodies may also be utilised, to include without limitation Fab fragments, ScFv, single domain antibodies, nanoantibodies, heavy chain antibodies etc which retain HDAC2 binding function. Any detection method may be employed in accordance with the invention. The nature of the reagent is not limited except that it must be capable of specifically identifying HDAC2.

Of course, in the case of a positive diagnostic of cancer, there will be reduced HDAC2 levels, and perhaps no HDAC2 at all. In one embodiment this will present a negative result, if the HDAC2 specific reagent is one which binds to the wild type or full length protein. In this case, use of suitable controls ensures that false diagnoses will not be made, for example caused by degraded or non-specific reagents. Thus, the same reagent can be tested on samples in which it is known that HDAC2 is expressed. A positive result in this control sample, combined with a negative result in the test sample would provide a confident diagnosis of cancer and removes any doubt over the quality of the reagent.

In one alternative embodiment, a reagent specific for the altered HDAC2 which is associated with cancer may be employed. Thus, for example, the truncated version of HDAC2 described herein is not recognised by reagents specific for full length and wild type HDAC2. Accordingly, this truncated version associated with cancer cells may fold differently and thus present different epitopes. As a result of this, reagents specific for truncated HDAC2 may be produced by known methods. A preferred reagent would comprise an antibody, or a truncated HDAC2 binding derivative thereof. Both monoclonal and polyclonal antibodies can be produced according to known methods and readily derivatized by one skilled in the art.

Use of such a reagent would allow a positive result to be used to diagnose cancer, since the reagent binds in the presence of the mutant HDAC2 which is indicative of a loss of wild type HDAC2 function and expression.

HDAC2 gene expression may also be monitored at the RNA level in one embodiment. Thus a decreased or abolished level of HDAC2 gene expression results in lower levels of functional HDAC2 protein and this is indicative of cancer.

Suitable methods for determining HDAC2 expression at the RNA level are well known in the art. Methods employing nucleic acid probe hybridization to the HDAC2 transcript may be employed for measuring the presence and/or level of HDAC2 mRNA. Such methods include use of nucleic acid probe arrays (microarray technology) and Northern blots. Advances in genomic technologies now permit the simultaneous analysis of thousands of genes, although many are based on the same concept of specific probe-target hybridization.

Sequencing-based methods are an alternative. These methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

In one embodiment, the levels of HDAC2 gene expression are determined using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

In a preferred embodiment, the RT-PCR is carried out in real time and in a quantitative manner. Real time quantitative RT-PCR has been thoroughly described in the literature (see Gibson et al for an early example of the technique) and a variety of techniques are possible. Examples include use of Taqman, Molecular Beacons, Lightcycler (Roche), Scorpion and Amplifluour systems. All of these systems are commercially available and well characterised, and may allow multiplexing (that is, the determination of expression of multiple genes in a single sample).

These techniques produce a fluorescent read-out that can be continuously monitored. Real-time techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the invention. This may be particularly important in the clinical setting of the present invention.

It should be noted that whilst PCR is a preferred amplification method, to include variants on the basic technique such as nested PCR, equivalents may also be included within the scope of the invention. Examples include isothermal amplification techniques such as NASBA, 3SR, TMA and triamplification, all of which are well known in the art and commercially available. Other suitable amplification methods include the ligase chain reaction (LCR) (Barringer et al, 1990), selective amplification of target polynucleotide sequences (U.S. Pat. No. 6,410,276), consensus sequence primed polymerase chain reaction (U.S. Pat. No. 4,437,975), arbitrarily primed polymerase chain reaction (WO90/06995) and nick displacement amplification (WO2004/067726).

The above referenced methods are also useful in embodiments of the methods of the invention in which the level or activity of HDAC2 is measured indirectly.

Thus, in one embodiment, the method of diagnosing cancer comprises determining the levels of gene expression of a panel of genes, comprising at least one gene, but preferably more than one gene selected from

(a) CARD6 (A_{—}23_P41854), TOP1MT (A_{—}23_P216241), SWAP70 (A_{—}23_P116533), KIAA1212 (A_{—}23_P17269), IRF5 (A_{—}23_P500271), CGI-90 (A_{—}23_P83492), UTS2 (A_{—}23_P63343), DJ462023.2 (A_{—}23_P46604), HHLA3 (A_{—}23_P200303), NFATC4 (A_{—}23_P140394), DKFZp686H1423 (A_{—}23_P392235), MGC17839 (A_{—}23_P329890), TSTA3 (A_{—}23_P94301), ZNF175 (A_{—}23_P332374), (A_{—}23_P119023), KIAA1212 (A_{—}23_P28664), LANCL1 (A_{—}23_P50887), FLJ38819 (A_{—}23_P385768), TAS2R9 (A_{—}23_P336506), ZNF22 (A_{—}23_P202458), SYT12 (A_{—}23_P424645), KIAA1826 (A_{—}23_P116166), ZHX1 (A_{—}23_P43150), BAIAP1 (A_{—}23_P96099), (A_{—}23_P150316), ZNF22 (A_{—}23_P384512), HMP19 (A_{—}23_P125375), M160 (A_{—}23_P61466), TFPI (A_{—}23_P17095), FJX1 (A_{—}23_P150693), (A_{—}23_P165598), EMP3 (A_{—}23_P119362), AP1S2 (A_{—}23_P217384), CCRL2 (A_{—}23_P69310), ZNF44 (A_{—}23_P119279), VAPB (A_{—}23_P91293), (A_{—}23_P28797), FLJ10458 (A_{—}23_P141315), FLJ10826 (A_{—}23_P3775), ZNF582 (A_{—}23_P395464), RBM9 (A_{—}23_P103091), C14orf168 (A_{—}23_P25913), KPNA4 (A_{—}23_P218835), FUNDC2 (A_{—}23_P171310), MGC29784 (A_{—}23_P255916), DEDD2 (A_{—}23_P141908), PHC1 (A_{—}23_P323094), CSTF1 (A_{—}23_P79882), SLIT2 (A_{—}23_P144348), EFHD2 (A_{—}23_P23443), MGC17986 (A_{—}23_P368777), ODF3L1 (A_{—}23_P357900), SGSH (A_{—}23_P254254), DEFB127 (A_{—}23_P385843), UCHL5 (A_{—}23_P148798), LTA4H (A_{—}23_P204593), MFN1 (A_{—}23_P253817), IKIP A_{—}23_P53467), LTA4H (A_{—}23_P388670), EIF3S6 (A_{—}23_P43141), GSDM1 (A_{—}23_P152605), ZZANK1 (A_{—}23_P72725), KPNA5 (A_{—}23_P156443), CHRAC1 (A_{—}23_P123544), FBXW2 (A_{—}23_P254645), PTGFRN (A_{—}23_P114968), DUSP19 (A_{—}23_P90938), PLEKHB2 (A_{—}23_P28642), TNFAIP6 (A_{—}23_P165624), (A_{—}23_P57836), C20orf45 (A_{—}23_P210658), PRPS1 (A_{—}23_P95764), PLSCR3 (A_{—}23_P49597), LOC92689 (A_{—}23_P132914), TRIM38 (A_{—}23_P93236), BRD4 (A_{—}23_P425104), KIAA0649 (A_{—}23_P146497), FBXO28 (A_{—}23_P137578), PPP2R4 (A_{—}23_P60458), IRAK1 (A_{—}23_P73780), SSH2 (A_{—}23_P118712), EIF5A (A_{—}23_P100925), C9orf103 (A_{—}23_P123732), EEF1D (A_{—}23_P31838), PRPS2 (A_{—}23_P96641), ABCC9 (A_{—}23_P368691), WDFY2 (A_{—}23_P218108), MGC75360 (A_{—}23_P163373), LTK (A_{—}23_P14853), (A_{—}23_P348015), ZNF571 (A_{—}23_P108342), FLJ12517 (A_{—}23_P115523), (A_{—}23_P90070), FLJ31951 (A_{—}23_P167556), MGC29875 (A_{—}23_P46337), KIAA0103 (A_{—}23_P60000), LARS (A_{—}23_P218967), PEX19 (A_{—}23_P160188), GPR83 (A_{—}23_P202740), ATAD2 (A_{—}23_P216068), ORSAK2 (A_{—}23_P1863), OR6Q1 (A_{—}23_P161891), ITR (A_{—}23_P88021), MGC41816 (A_{—}23_P95027), NDUFB9 (A_{—}23_P157669), HSF1 (A_{—}23_P253841), FAM48A (A_{—}23_P140050), RPL10L (A_{—}23_P99754), KIAA1223 (A_{—}23_P81012), RRM2B (A_{—}23_P20223), LOC90806 (A_{—}23_P97697), LY6E (A_{—}23_P169077), FLII (A_{—}23_P4425), IL3 (A_{—}23_P122012), FLJ21168 (A_{—}23_P85993), BCL2L2 (A_{—}23_P140219), PIG8 (A_{—}23_P86822), SMARCA1 (A_{—}23_P44244), SCUBE1 (A_{—}23_P211619), CDYL2 (A_{—}23_P371871), GDPD1 (A_{—}23_P96060), SERAC1 (A_{—}23_P145419), C11orf23 (A_{—}23_P202637), FKBP10 (A_{—}23_P15727), WDR35 (A_{—}23_P108463), PPM1K (A_{—}23_P167138), NUDT12 (A_{—}23_P259090), DIAPH1 (A_{—}23_P213344), SIGIRR (A_{—}23_P84344), SCGN (A_{—}23_P251412), CTNNAL1 (A_{—}23_P157795), DIPA (A_{—}23_P150249), SGPP1 (A_{—}23_P347048), LOC196549 (A_{—}23_P311039), KREMEN2 (A_{—}23_P77612), FMNL2 (A_{—}23_P332744),

(b) ACP6 (A_{—}23_P160237), C3orf17 (A_{—}23_P336670), SLC10A5 (A_{—}23_P157428), (A_{—}23_P60758), ALG6 (A_{—}23_P35168), MAP4K3 (A_{—}23_P154130), ITLN2 (A_{—}23_P201419), PPIL4 (A_{—}23_P42507), RAE1 (A_{—}23_P346206), DDX20 (A_{—}23_P63153), CMKLR1 (A_{—}23_P105461), GRM2 (A_{—}23_P252184), DC12 (A_{—}23_P60947), (A_{—}23_P113983), PTPLB (A_{—}23_P155197), KIAA1639 (A_{—}23_P103734), MAD2L2 (A_{—}23_P23206), FLJ35382 (A_{—}23_P304410), OTOR (A_{—}23_P403385), MRPL47 (A_{—}23_P502427), (A_{—}23_P73451), CASC1 (A_{—}23_P95231), LOC128387 (A_{—}23_P23522), SBBI54 (A_{—}23_P67323), EDEM1 (A_{—}23_P40975), YARS (A_{—}23_P85570), CSNK2A2 (A_{—}23_P14915), MC1R (A_{—}23_P66095), MDS025 (A_{—}23_P162127), C14orf118 (A_{—}23_P54198), NIFIE14 (A_{—}23_P79043), (A_{—}23_P44514), OR5T1 (A_{—}23_P47444), TMF1 (A_{—}23_P143867), KNS2 (A_{—}23_P2873), (A_{—}23_P256903), LLGL1 (A_{—}23_P130266), (A_{—}23_P159277), C10orf117 (A_{—}23_P202496), TRAPPC3 (A_{—}23_P200535), SERPINA12 (A_{—}23_P88177), ARPC1A (A_{—}23_P82664), GTF2A1 (A_{—}23_P65609), ANKRD16 (A_{—}23_P150069), PTN (A_{—}23_P303085), FBXL20 (A_{—}23_P141146), AMPD2 (A_{—}23_P201591), CHRNG (A_{—}23_P17115), FLJ20507 (A_{—}23_P100155), FLJ35036 (A_{—}23_P212025), ARHGAP22 (A_{—}23_P75310), SENP2 (A_{—}23_P257988), LOC401137 (A_{—}23_P133011), BBS4 (A_{—}23_P99961), ETEA (A_{—}23_P156109), ITPKB (A_{—}23_P372255), TBC1D1 (A_{—}23_P73023), (A_{—}23_P147154), HSRG1 (A_{—}23_P206400), ZNF568 (A_{—}23_P67737), HCRTR1 (A_{—}23_P74178), LIPT1 (A_{—}23_P501805), HOXB3 (A_{—}23_P100872), GP9 (A_{—}23_P33256), XRN1 (A_{—}23_P132820), SF3A1 (A_{—}23_P104680), SCO1 (A_{—}23_P15466), GRIN2B (A_{—}23_P151264), Ufc1 (A_{—}23_P103905), PHF17 (A_{—}23_P167256), SF4

(A_{—}23_P119618), PLAA (A_{—}23_P135157), NDUFA11 (A_{—}23_P320185), RPL10L (A_{—}23_P341325), F8 (A_{—}23_P217643), SAGE1 (A_{—}23_P33343), ZFP28 (A_{—}23_P107673), MGC21654 (A_{—}23_P334218), (A_{—}23_P119652), FLJ11305 (A_{—}23_P25644), TRIP3 (A_{—}23_P383435), RHOF (A_{—}23_P203983), OR5AR1 (A_{—}23_P13273), C5orf3 (A_{—}23_P41912), EIF2B4 (A_{—}23_P154056), CPSF3 (A_{—}23_P28683), FLJ10774 (A_{—}23_P87329), GLUD1 (A_{—}23_P138665), KBTBD4 (A_{—}23_P202693), FLJ33167 (A_{—}23_P358470), RAB8B (A_{—}23_P37535), PAX9 (A_{—}23_P426944), SURF5 (A_{—}23_P501795), (A_{—}23_P46708), OR12D3 (A_{—}23_P259555), C9orf80 (A_{—}23_P397899), MAFF (A_{—}23_P103110), RND1 (A_{—}23_P53370), NPPA (A_{—}23_P74059), C9orf77 (A_{—}23_P83149), ATP6VOD1 (A_{—}23_P54636), IKBKB (A_{—}23_P216188), GUK1 (A_{—}23_P201097), COH1 (A_{—}23_P20298), NFATC3 (A_{—}23_P77440), C20orf128 (A_{—}23_P409888), CHES1 (A_{—}23_P106236), (A_{—}23_P86369), KRTHB1 (A_{—}23_P363769), LEP (A_{—}23_P31426), (A_{—}23_P136857), SERPINB6 (A_{—}23_P70348), FLJ11004 (A_{—}23_P118042), TDRD7 (A_{—}23_P123672), OR1C1 (A_{—}23_P86350), FBXO11 (A_{—}23_P90814), ST13 (A_{—}23_P68949), PRKAG1 (A_{—}23_P36513), PHKA1 (A_{—}23_P258531), FLJ32731 (A_{—}23_P112061), (A_{—}23_P119788), PSG3 (A_{—}23_P56354), PTPRT (A_{—}23_P135576), MOCOS (A_{—}23_P67042), SDSL (A_{—}23_P53439), FLJ21816 (A_{—}23_P129569), KATNA1 (A_{—}23_P113803), BRDT (A_{—}23_P147976), FNTA (A_{—}23_P24926), RAB3B (A_{—}23_P62672), CTAG1B (A_{—}23_P148541), GLE1L (A_{—}23_P258367), ADCY7 (A_{—}23_P106949), ABI1 (A_{—}23_P126992), TCEA1 (A_{—}23_P132444), BTBD5 (A_{—}23_P205659), (A_{—}23_P149398), (A_{—}23_P158524), CDCA4 (A_{—}23_P205449), NUTF2 (A_{—}23_P118038), OR1B1 (A_{—}23_P146611), PDE6B (A_{—}23_P10743), HSC20 (A_{—}23_P40588), RNF8 (A_{—}23_P70384), (A_{—}23_P254382), ALDH9A1 (A_{—}23_P11884), BCL6B (A_{—}23_P130130), PPP1R12C (A_{—}23_P50575), GCM1 (A_{—}23_P133728), ARHGAP17 (A_{—}23_P3716), C9orf10 (A_{—}23_P32294), DR1 (A_{—}23_P63205), SPR (A_{—}23_P68208), RAB9P40 (A_{—}23_P123814), TTC19 (A_{—}23_P164421), PRKAA1 (A_{—}23_P110725), LOC127253 (A_{—}23_P103180), FANCE (A_{—}23_P42335), BMP15 (A_{—}23_P11107), HSPG2 (A_{—}23_P23191), POLD1 (A_{—}23_P50456), SLC3A1 (A_{—}23_P165325), PRUNE (A_{—}23_P169925), SF3B3 (A_{—}23_P135914), OXT (A_{—}23_P57133), EEF1G (A_{—}23_P13344), MGC2494 (A_{—}23_P54728), SUV39H2 (A_{—}23_P202392), WDR3 (A_{—}23_P23318), PLP2 (A_{—}23_P251089), GRM5 (A_{—}23_P24361), FLJ14007 (A_{—}23_P146050), GFER (A_{—}23_P206484), LASS4 (A_{—}23_P153867), C9orf16 (A_{—}23_P158048), ITIH1 (A_{—}23_P18223), CYP17A1 (A_{—}23_P75111), ACTN3 (A_{—}23_P138881), FLJ39378 (A_{—}23_P303293), GALNACT-2 (A_{—}23_P149887), COBRA1 (A_{—}23_P148150), COPS8 (A_{—}23_P144257), LOC1550.60 (A_{—}23_P254441), DGKI (A_{—}23_P111432), SLC22A6 (A_{—}23_P98616), SH3BP2 (A_{—}23_P110116), RTTN (A_{—}23_P337896), LOC51255 (A_{—}23_P108708), FLNA (A_{—}23_P96559), HSD17B12 (A_{—}23_P47377), EBAG9 (A_{—}23_P255226), PPP1R16A (A_{—}23_P157715), FIBP (A_{—}23_P13018), SH3GLB2 (A_{—}23_P216995), PHF20L1 (A_{—}23_P134786), C10orf9 (A_{—}23_P434-419), CEP1 (A_{—}23_P32183), NDUFV2 (A_{—}23_P130418), LOC147804 (A_{—}23_P55854), TOMM34 (A_{—}23_P57033), MSL3L1 (A_{—}23_P217776), TOPORS (A_{—}23_P32217), S100A13 (A_{—}23_P372874), GBA (A_{—}23_P201030), C21orf55 (A_{—}23_P68700), IFNGR2 (A_{—}23_P29034), SNAPC1 (A_{—}23_P37251), OR56B1 (A_{—}23_P139244), APOA1BP (A_{—}23_P126446), SUHW3 (A_{—}23_P314642), C2orf33 (A_{—}23_P405148), SSSCA1 (A_{—}23_P98261), CKLFSF7 (A_{—}23_P256413), NBEAL1 (A_{—}23_P217068), (A_{—}23_P49725), ZNF45 (A_{—}23_P426-472), LOC400986 (A_{—}23_P131322), CHURC1 (A_{—}23_P37237), GSS (A_{—}23_P210920), CD96 (A_{—}23_P32770), C21orf61 (A_{—}23_P57293), DERL1 (A_{—}23_P216043), ACAD8 (A_{—}23_P47426), MGC14595 (A_{—}23_P157679), ALDOA (A_{—}23_P88961), SLC39A4 (A_{—}23_P20502), JRK (A_{—}23_P8930), AAMP (A_{—}23_P56529), FERD3L (A_{—}23_P422849), PRKCSH (A_{—}23_P208615), TCTE1L (A_{—}23_P73577), EPHB1 (A_{—}23_P166994), FAM49B (A_{—}23_P43255), HSU79274 (A_{—}23_P65000), PRDX6 (A_{—}23_P983), LOC400986 (A_{—}23_P2.10134), ARL10C (A_{—}23_P212229), OSGEP (A_{—}23_P54078), LOR (A_{—}23_P22297), BCKDK (A_{—}23_P365149),

(c) PTPN1 (A_{—}23_P338890), RBKS (A_{—}23_P9523), DEFB119 (A_{—}23_P413089), PARP11 (A_{—}23_P343837), SLC12A1 (A_{—}23_P99879), CEP2 (A_{—}23_P102832), CREB5 (A_{—}23_P157117), GPX5 (A_{—}23_P214544), STATH (A_{—}23_P252253), MGC2474 (A_{—}23_P3819), AP2A1 (A_{—}23_P164889), KLF15 (A_{—}23_P40809), ZMYND19 (A_{—}23_P305173), EIF3S4 (A_{—}23_P38887), COL8A1 (A_{—}23_P80436), IFIT5 (A_{—}23_P63668), NCB50R (A_{—}23_P30995), DMRT2 (A_{—}23_P365694), (A_{—}23_P2180), TRY1 (A_{—}23_P82558), DVL1 (A_{—}23_P347432), CNDP1 (A_{—}23_P9869), ADA (A_{—}23_P210482), LENG1 (A_{—}23_P208520), (A_{—}23_P25534), STX4A (A_{—}23_P256375), FLJ11171 (A_{—}23_P106753), CAPG (A_{—}23_P165636), BNC2 (A_{—}23_P43684), LOC387758 (A_{—}23_P138885), NCE2 (A_{—}23_P39561), MDGA1 (A_{—}23_P310460), OTUB1 (A_{—}23_P24375), FLJ38348 (A_{—}23_P339633), ZBTB9 (A_{—}23_P8116), CAP2 (A_{—}23_P156417), OR1S1 (A_{—}23_P52957), CLCA3 (A_{—}23_P34382), (A_{—}23_P94020), IRAK2 (A_{—}23_P80635), CITED4 (A_{—}23_P74155), FLJ21369 (A_{—}23_P50597), POLE4 (A_{—}23_P154234), SH3GLB1 (A_{—}23_P22957), PROL5 (A_{—}23_P41365), DKFZp547E052 (A_{—}23_P102048), SLC4A7 (A_{—}23_P132468), LETM2 (A_{—}23_P348264), RABL3 (A_{—}23_P369393), OR51T1 (A_{—}23_P24856), DNCI2 (A_{—}23_P154108), FLJ20534 (A_{—}23_P60510), FLJ10979 (A_{—}23_P130285), (A_{—}23_P135946), WASF3 (A_{—}23_P151351), PSG6 (A_{—}23_P108170), CDY1 (A_{—}23_P62471), TNFRSF12A (A_{—}23_P49338), C10orf97 (A_{—}23_P23983), CPSF5 (A_{—}23_P129614), FLJ38944 (A_{—}23_P368259), Bles03 (A_{—}23_P150238), ADRB3 (A_{—}23_P168993), DNAJC8 (A_{—}23_P37137), SNX17 (A_{—}23_P28233), MSR1 (A_{—}23_P216176), ARL8 (A_{—}23_P378588), (A_{—}23_P71709), RRN3 (A_{—}23_P206877), SLN (A_{—}23_P150343), C1QTNF4 (A_{—}23_P52597), SCARF1 (A_{—}23_P15414), NUDT5 (A_{—}23_P1196), ELOVL6 (A_{—}23_P7361), RAD54B (A_{—}23_P94141), C3orf20 (A_{—}23_P361333), OLFML1 (A_{—}23_P147664), ST7L (A_{—}23_P310582), (A_{—}23_P169587), POLE4 (A_{—}23_P383385), PFDN2 (A_{—}23_P51906), FBXL6 (A_{—}23_P43296), MYC (A_{—}23_P215956), FMO1 (A_{—}23_P12386), KIAA1446 (A_{—}23_P253614), HSPA4L (A_{—}23_P363936), DCP2 (A_{—}23_P256868), THRAP6 (A_{—}23_P31866), (A_{—}23_P158699), DNAJB4 (A_{—}23_P51339), LECT2 (A_{—}23_P110777), ZNF45 (A_{—}23_P101721), KCNQ2 (A_{—}23_P210400), CALML5 (A_{—}23_P124095), RAD23A (A_{—}23_P55889), FLJ32784 (A_{—}23_P406-478), FLJ13220 (A_{—}23_P155959), FANCF (A_{—}23_P12896), (A_{—}23_P124703), GCDH (A_{—}23_P90089), CYP27A1 (A_{—}23_P131308), LIPG (A_{—}23_P78405), APBA2BP (A_{—}23_P68628), FBXL4 (A_{—}23_P214739), PSMD5 (A_{—}23_P43434), RGS11 (A_{—}23_P118124), LTB4DH (A_{—}23_P157809), FLJ20557 (A_{—}23_P55688), JARID1B (A_{—}23_P409866), (A_{—}23_P133049), QDPR (A_{—}23_P167212), NCOA4 (A_{—}23_P86424), ZNF25 (A_{—}23_P381577), FLJ21934 (A_{—}23_P213279), RP1 (A_{—}23_P71368), FLJ23790 (A_{—}23_P411215), TRIM3 (A_{—}23_P150619), C9orf27 (A_{—}23_P123702), C20orf43 (A_{—}23_P143258), PRPS1L1 (A_{—}23_P418516), MGC2655 (A_{—}23_P37949), OR1N1 (A_{—}23_P169376), EIF2C2 (A_{—}23_P112159), ALS2CR2 (A_{—}23_P90682), RBPMS (A_{—}23_P71316), RBMS2 (A_{—}23_P36432), (A_{—}23_P65843), LOC51236 (A_{—}23_P61268), (A_{—}23_P20372), EIF3S3 (A_{—}23_P9061), OGFRL1 (A_{—}23_P7791), FLJ31547 (A_{—}23_P118234), CPXM2 (A_{—}23_P138524), ZNF382 (A_{—}23_P16354), IRX2 (A_{—}23_P156025), DKFZP56400463 (A_{—}23_P215875), (A_{—}23_P340640), (A_{—}23_P80788), FLJ10178 (A_{—}23_P96369), (A_{—}23_P32966), SCHIP1 (A_{—}23_P257031), SLC6A6 (A_{—}23_P69206), C2orf3 (A_{—}23_P120064), OR1K1 (A_{—}23_P251528), CD207 (A_{—}23_P39790), THSD6 (A_{—}23_P5246), HIST1H2AI (A_{—}23_P168008), TAS2R16 (A_{—}23_P59825), SOAT1 (A_{—}23_P63319), NET-5 (A_{—}23_P151127), HPCAL1 (A_{—}23_P5831), C4BPA (A_{—}23_P97541), TBCD (A_{—}23_P100602), CTAG3 (A_{—}23_P361509), DEFB125 (A_{—}23_P320846), FLJ23588 (A_{—}23_P68978), CXorf1 (A_{—}23_P430747), DAPK1 (A_{—}23_P252163), SIAT2 (A_{—}23_P131455), (A_{—}23_P84475), DBCCR1L (A_{—}23_P103812), TNFRSF10D (A_{—}23_P95417), CORO1C (A_{—}23_P53456), OR11H1 (A_{—}23_P128866), FLJ32731 (A_{—}23_P147950), ERCC1 (A_{—}23_P107701), ZNF550 (A_{—}23_P354827), IQCG (A_{—}23_P124381), DYRK3 (A_{—}23_P12282),

(d) ASB11 (A_{—}23_P114259), (A_{—}23_P81273), OR52A1 (A_{—}23_P125286), LOXHD1 (A_{—}23_P107531), SPTBN2 (A_{—}23_P98282), CNNM2 (A_{—}23_P24044), ZNF71 (A_{—}23_P119068), PITPNB (A_{—}23_P17786), ANAPC2 (A_{—}23_P253062), SLC32A1 (A_{—}23_P154561), C20orf178 (A_{—}23_P28964), TSP-NY (A_{—}23_P2258), FLJ12525 (A_{—}23_P256021), ABO (A_{—}23_P112645), ADCK4 (A_{—}23_P208409), FLJ22405 (A_{—}23_P69229), (A_{—}23_P169341), PTHR2 (A_{—}23_P28181), LOC51058 (A_{—}23_P97221), (A_{—}23_P72359), RUTBC3 (A_{—}23_P166491), EXOSC7 (A_{—}23_P58102), FOXD3 (A_{—}23_P46560), ERF (A_{—}23_P4678), MCOLN1 (A_{—}23_P27571), FLJ38377 (A_{—}23_P313010), CD40 (A_{—}23_P57036), RPP38 (A_{—}23_P150080), NUP160 (A_{—}23_P43726), CLC (A_{—}23_P101684), RPP21 (A_{—}23_P214594), HSPA6 (A_{—}23_P114903), MAP3K11 (A_{—}23_P98273), TBX15 (A_{—}23_P62981), STAT5B (A_{—}23_P100788), (A_{—}23_P23673), EAP30 (A_{—}23_P130064), (A_{—}23_P133087), RYK (A_{—}23_P388081), RCP9 (A_{—}23_P361542), RYK (A_{—}23_P22001), SCN4B (A_{—}23_P303833), DUSP10 (A_{—}23_P51861), ACTL7B (A_{—}23_P9186), OLIG2 (A_{—}23_P211079), CT120 (A_{—}23_P50000), (A_{—}23_P33326), FARSLA (A_{—}23_P78682), (A_{—}23_P69129), CAPN14 (A_{—}23_P56450), PLA2G2E (A_{—}23_P114857), C21orf86 (A_{—}23_P417294), TTLL2 (A_{—}23_P7901), CYP2W1 (A_{—}23_P31437), CCR8 (A_{—}23_P211699), ZNFN1A5 (A_{—}23_P35667), FLJ35155 (A_{—}23_P345708), PPP2CB (A_{—}23_P308097), KIAA0100 (A_{—}23_P207614), (A_{—}23_P114895), DKFZP586A0522 (A_{—}23_P128432), MAK (A_{—}23_P214839), PS1D (A_{—}23_P96976), EMR4 (A_{—}23_P44380), SYTL4 (A_{—}23_P11136), DHODH (A_{—}23_P15202), LOC116441 (A_{—}23_P91885), DNAJC4 (A_{—}23_P202769), XPR1 (A_{—}23_P63534), POLR2C (A_{—}23_P3527), SFXN4 (A_{—}23_P387722), CRK7 (A_{—}23_P350093), KCNJ15 (A_{—}23_P17655), FBXO11 (A_{—}23_P79416), SLC22A9 (A_{—}23_P150590), CACNG5 (A_{—}23_P66497), IL1RAPL2 (A_{—}23_P255038), MRAS (A_{—}23_P377197), MGC16733 (A_{—}23_P203737), FLJ38716 (A_{—}23_P343366), TRA@ (A_{—}23_P106061), MGC11134 (A_{—}23_P98244), SEC10L1 (A_{—}23_P14464), SEZ6 (A_{—}23_P389569), GRPEL1 (A_{—}23_P92476), TOSO (A_{—}23_P138125), FBXL12 (A_{—}23_P78554), ACTA1 (A_{—}23_P1102), FGFR4 (A_{—}23_P92754), TCBA1 (A_{—}23_P170209), FLJ10246 (A_{—}23_P144704), DEAF1 (A_{—}23_P158533), (A_{—}23_P29296), SLC9A8 (A_{—}23_P252936), MS4A6E (A_{—}23_P416234), SELK (A_{—}23_P364517), SCGB1D4 (A_{—}23_P98598), TM9SF4 (A_{—}23_P210872), DHX32 (A_{—}23_P47004), PRPSAP2 (A_{—}23_P49646), HELB (A_{—}23_P2294), LTC4S (A_{—}23_P92802), PGRMC2 (A_{—}23_P155868), BLZF1 (A_{—}23_P23266), NDFIP1 (A_{—}23_P81247), SUPT6H (A_{—}23_P141479), CECR6 (A_{—}23_P259344), CNGA4 (A_{—}23_P2006), C14orf119 (A_{—}23_P117447), (A_{—}23_P17984), PAX7 (A_{—}23_P126225), DR1 (A_{—}23_P391725), XAB1 (A_{—}23_P17144), BMP8A (A_{—}23_P126508), C1orf26 (A_{—}23_P96931), TRPM1 (A_{—}23_P129225), ALG3 (A_{—}23_P7005), OR5D14 (A_{—}23_P36057), C2orf29 (A_{—}23_P108761), MGC26717 (A_{—}23_P44177), SHBG (A_{—}23_P207088), RAMP3 (A_{—}23_P111737), OR1J2 (A_{—}23_P157996), CLSPN (A_{—}23_P126207), MOV10 (A_{—}23_P161125), (A_{—}23_P155997), MS4A8B (A_{—}23_P139147), BCAR1 (A_{—}23_P77724), SSX4 (A_{—}23_P254202), FLJ14129 (A_{—}23_P60101), HDAC8 (A_{—}23_P84922), ORC3L (A_{—}23_P42045), GFRA2 (A_{—}23_P84084), AFG3L2 (A_{—}23_P135454), GPR45 (A_{—}23_P131534), PBOV1 (A_{—}23_P255942), PRKCE (A_{—}23_P250564), PPP2CZ (A_{—}23_P201939), DAP3 (A_{—}23_P63067), CTNS (A_{—}23_P427075), SMARCA5 (A_{—}23_P256716), FRS2 (A_{—}23_P430785), CROCC (A_{—}23_P104086), PLK4 (A_{—}23_P155968), CSIG (A_{—}23_P54597), SLC35F2 (A_{—}23_P339098), NT5C2L1 (A_{—}23_P382043), IL3 (A_{—}23_P348828), ZNF434 (A_{—}23_P218283), KIR2DS5 (A_{—}23_P381532), (A_{—}23_P72169), GML (A_{—}23_P59976), RBM7 (A_{—}23_P138975), SIGLEC7 (A_{—}23_P50182), SRPK2 (A_{—}23_P215594), B4GALT3 (A_{—}23_P103912), XRN2 (A_{—}23_P166135), PARP4 (A_{—}23_P117172), PTBP1 (A_{—}23_P208981), (A_{—}23_P33148), GPRC6A (A_{—}23_P81683), KIAA0063 (A_{—}23_P6479), WDR12 (A_{—}23_P28625), HIRIP5 (A_{—}23_P147296), ARHGAP27 (A_{—}23_P141336), DONSON (A_{—}23_P500390), PRDM9 (A_{—}23_P7612), ATP7B (A_{—}23_P205228), IL15RA (A_{—}23_P138680), (A_{—}23_P166910), FLJ35801 (A_{—}23_P433719), MAPK14 (A_{—}23_P214696), GALE (A_{—}23_P160148), OVCH1 (A_{—}23_P435974), (A_{—}23_P170925), LRCH2 (A_{—}23_P73809), HAL (A_{—}23_P61643), (A_{—}23_P164520), CNOT4 (A_{—}23_P156993), SIRT2 (A_{—}23_P142455), KIAA1984 (A_{—}23_P302410), ZNF497 (A_{—}23_P27414), (A_{—}23_P84555), CCNB3 (A_{—}23_P171107), SLC8A1 (A_{—}23_P119957), TUSC4 (A_{—}23_P18267), ZP1 (A_{—}23_P1912), AKR7A3 (A_{—}23_P103968), ZCWCC3 (A_{—}23_P325501), RAB7L1 (A_{—}23_P126939), GP6 (A_{—}23_P27784), ZNF394 (A_{—}23_P157416), RPS9 (A_{—}23_P208535), GTF2H1 (A_{—}23_P36183), GPR135 (A_{—}23_P205575), SLC6A16 (A_{—}23_P130735), HKE2 (A_{—}23_P122563), TUBG1 (A_{—}23_P152768), ARD1 (A_{—}23_P148546), STAMBP (A_{—}23_P28555), (A_{—}23_P154166), RPL10 (A_{—}23_P217666), ROPN1L (A_{—}23_P121885), PAK3 (A_{—}23_P346813), ZNF235 (A_{—}23_P208325), RBBP9 (A_{—}23_P57181), EXOSC9 (A_{—}23_P81121), STK25 (A_{—}23_P252653), (A_{—}23_P94711), ZNF485 (A_{—}23_P115861), WBSCR18 (A_{—}23_P157168), ENDOG (A_{—}23_P83266), RBL1 (A_{—}23_P28733), C21orf70 (A_{—}23_P61202), (A_{—}23_P210784), (A_{—}23_P98669), KCNJ6 (A_{—}23_P29058), USP32 (A_{—}23_P107154), (A_{—}23_P31829), C21orf129 (A_{—}23_P413696), SLC31A1 (A_{—}23_P253409), ZNF192 (A_{—}23_P214529), OR3A2 (A_{—}23_P55504), CLNS1A (A_{—}23_P127995), ZFP106 (A_{—}23_P77310), USP47 (A_{—}23_P148204), IDI2 (A_{—}23_P52543), PTPN3 (A_{—}23_P403898), AUP1 (A_{—}23_P253421), THG-1 (A_{—}23_P250865), MKLN1 (A_{—}23_P93613), (A_{—}23_P29866), LOC90353 (A_{—}23_P303320), FLJ20729 (A_{—}23_P201396), (A_{—}23_P16753), PGD (A_{—}23_P126623), TNRC15 (A_{—}23_P142950), SMYD5 (A_{—}23_P91015), EHBP1L1 (A_{—}23_P148114), PAF53 (A_{—}23_P9455), TCEB2 (A_{—}23_P312181), NSUN6 (A_{—}23_P61580), WNT7A (A_{—}23_P258410), P29 (A_{—}23_P45756), BTBD14A (A_{—}23_P113825), GALNT2 (A_{—}23_P806), CD2AP (A_{—}23_P156484), FLJ39378 (A_{—}23_P169934), SUV39H1 (A_{—}23_P422193), IL24 (A_{—}23_P51951), HOXC6 (A_{—}23_P150974), SLC17A2 (A_{—}23_P59048), GLUD2 (A_{—}23_P45361), CDCl₄A (A_{—}23_P201921), UBE2A (A_{—}23_P148446), RANGNRF (A_{—}23_P55136), RANBP3 (A_{—}23_P208973), TUFM (A_{—}23_P251209), NLGN3 (A_{—}23_P62303), PACSIN2 (A_{—}23_P80377), DGCR8 (A_{—}23_P211355), EMD (A_{—}23_P85171), HERC4 (A_{—}23_P202408), EEF2 (A_{—}23_P5027), KIAA1754 (A_{—}23_P340333), (A_{—}23_P112825), HEBP1 (A_{—}23_P117082), SNRPB (A_{—}23_P154675), BRUNOL5 (A_{—}23_P60766), C6orf89 (A_{—}23_P396842), JMJD1C A_{—}23_P86434), MGC42638 (A_{—}23_P367071), HMG20B (A_{—}23_P119543), C9orf98 (A_{—}23_P83200), (A_{—}23_P89012), SCRT1 (A_{—}23_P147782), PLXNA4 (A_{—}23_P111448), MYH13 (A_{—}23_P89334), GCET2 (A_{—}23_P253245), RNF20 (A_{—}23_P216850), ZDHHC13 (A_{—}23_P13065), PPP4C (A_{—}23_P100355), OR1L8 (A_{—}23_P157991), UBQLN3 (A_{—}23_P98830), SH3BGR (A_{—}23_P91520), DPAGT1 (A_{—}23_P1775), FLJ35740 (A_{—}23_P310552), LOC51333 (A_{—}23_P416836), COH1 (A_{—}23_P359173), ATOH1 (A_{—}23_P332246), ZG16 (A_{—}23_P49145), E2F7 (A_{—}23_P322426), STAM (A_{—}23_P1343), ATP2B2 (A_{—}23_P18153), HLA-DOA (A_{—}23_P30923), HT007 (A_{—}23_P2124), 1HPK1 (A_{—}23_P250537), SCIRP10 (A_{—}23_P372434), MYOZ1 (A_{—}23_P1320), SEZ6L (A_{—}23_P80242), LCMT1 (A_{—}23_P124213), C21orf61 (A_{—}23_P304554), ALPL (A_{—}23_P97043), OAZIN (A_{—}23_P157435), SLC34A2 (A_{—}23_P121646), ZNF505 (A_{—}23_P135826), AQP9 (A_{—}23_P106362), HTR3B (A_{—}23_P87007), RABGEF1 (A_{—}23_P250824), GPRC5D (A_{—}23_P105691), (A_{—}23_P305581), OR51A4 (A_{—}23_P218015), TRPC4 (A_{—}23_P105873), (A_{—}23_P113263), OR51B6 (A_{—}23_P47774), SLC36A1 (A_{—}23_P167640), AGTRAP (A_{—}23_P147215), C20orf30 (A_{—}23_P17482), (A_{—}23_P146679), DSC1 (A_{—}23_P38696), POLR3A (A_{—}23_P149826), FIS (A_{—}23_P408323), RUVBL2 (A_{—}23_P39110), ELAVL1 (A_{—}23_P388681), KCNQ3 (A_{—}23_P123393), GPR119 (A_{—}23_P21425), SEC10L1 (A_{—}23_P413826), TMEM7 (A_{—}23_P57910), C20orf116 (A_{—}23_P403968), GGN (A_{—}23_P333552), OR8S1 (A_{—}23_P53378), NEK3 (A_{—}23_P76718), FGF1 (A_{—}23_P213334), WDR1 (A_{—}23_P213001), MAFG (A_{—}23_P207759), PLB1 (A_{—}23_P56356), BAK1 (A_{—}23_P145357), KCNA10 (A_{—}23_P126528), HPRP8BP (A_{—}23_P45913), C6orf29A_{—}23_P93352), DBT (A_{—}23_P74022), NCOA61P (A_{—}23_P60899), MTNR1A (A_{—}23_P80987), PSMB2 (A_{—}23_P170058), ASAHL (A_{—}23_P155666), PSMD11 (A_{—}23_P207600), ANP32C (A_{—}23_P92524), RBM16 (A_{—}23_P111303), LOC126295 (A_{—}23_P39263), IMP4 (A_{—}23_P302094), ADAM2 (A_{—}23_P73441), EBNA1BP2 (A_{—}23_P103631), MYOZ3 (A_{—}23_P58686), LOC92912 (A_{—}23_P77274), UMP-CMPK A_{—}23_P115366), KIAA0748 (A_{—}23_P105423), CRYGB (A_{—}23_P414842), GDAP1L1 (A_{—}23_P17356), MUS81 (A_{—}23_P161634), FLJ30656 (A_{—}23_P307430), KCNN4 (A_{—}23_P67529), SLC16A1 (A_{—}23_P126825), DNAJC5B (A_{—}23_P8959), CGI-94 (A_{—}23_P11774), DPT (A_{—}23_P200741), RHPN1 (A_{—}23_P87438), CRIM1 (A_{—}23_P51105), SPP2 (A_{—}23_P90829), (A_{—}23_P73264), STAT6 (A_{—}23_P47879), IL22 (A_{—}23_P117115), LOC142937 (A_{—}23_P402164), SMYD3 (A_{—}23_P51414), FLJ10808 (A_{—}23_P218905), FLJ20184 (A_{—}23_P252276), WNT7B (A_{—}23_P147544), NARG1 (A_{—}23_P212825), MGC10067 (A_{—}23_P400344), SEC22L2 (A_{—}23_P259874), (A_{—}23_P81092), LOC285590 (A_{—}23_P122104), PROK1 (A_{—}23_P51745), TSSC4 (A_{—}23_P2023), EYA1 (A_{—}23_P502363), ARL2L1 (A_{—}23_P10081), TXNDC (A_{—}23_P309711), TNFSF8 (A_{—}23_P169257), BRAP (A_{—}23_P53614), SERPINH1 (A_{—}23_P75998), RASAL2 (A_{—}23_P86124), PRKDC (A_{—}23_P169612), PDE7B (A_{—}23_P59338), MRPL4 (A_{—}23_P164702), HTR2B (A_{—}23_P16953), NDUFB7 (A_{—}23_P50872), TUFM (A_{—}23_P9582), NIT2 (A_{—}23_P110099), PLK3 (A_{—}23_P51646), FLJ11267 (A_{—}23_P168965), LOC55971 (A_{—}23_P168470), CPSF6 (A_{—}23_P204039), PAQR4 (A_{—}23_P66213), MT (A_{—}23_P132358), EIF3S5 (A_{—}23_P142774), CRIPT (A_{—}23_P17219), APOBEC3B (A_{—}23_P369966), CBX6 (A_{—}23_P40677), ANKRD13 (A_{—}23_P36738), KIAA1900 (A_{—}23_P252521), AURKB (A_{—}23_P130182), (A_{—}23_P36439), RAB13 (A_{—}23_P46365), DUSP24 (A_{—}23_P111635), FLJ40137 (A_{—}23_P55196), SARS (A_{—}23_P126790), FLJ32830 (A_{—}23_P430788), N-PAC (A_{—}23_P396785), FLJ35700 (A_{—}23_P342165), MGC24039 (A_{—}23_P379746), TLK1 (A_{—}23_P39684), M1-ER1 (A_{—}23_P305723), PAQR3 (A_{—}23_P167276), ACTR2 (A_{—}23_P377376), PSFL (A_{—}23_P205997), CLIC4 (A_{—}23_P259189), CCRN4L (A_{—}23_P144338), IMPA1 (A_{—}23_P168818), FLJ20989 (A_{—}23_P33113), LOC124446 (A_{—}23_P77562), RPL28 (A_{—}23_P208356), BANF1 (A_{—}23_P47210), RAB37 (A_{—}23_P163972), (A_{—}23_P22639), TCBA1 (A_{—}23_P316501), HADHA (A_{—}23_P159510), WDR5 (A_{—}23_P32558), FLJ12442 (A_{—}23_P44836), DKFZP547N043 (A_{—}23_P433791), MPV17 (A_{—}23_P143084), KIAA0256 (A_{—}23_P37416), (A_{—}23_P158837), ADAMTS5 (A_{—}23_P40415), PTD008 (A_{—}23_P218486), UPK2 (A_{—}23_P64243), TERF1 (A_{—}23_P216149), STIP1 (A_{—}23_P124470), LASS5 (A_{—}23_P76515), OR5T3 (A_{—}23_P75710), TMED5 (A_{—}23_P86002), CCR4 (A_{—}23_P72989), (A_{—}23_P131887), (A_{—}23_P158605), AATF (A_{—}23_P89460), PME-1 (A_{—}23_P64567), (A_{—}23_P57626), RBM8A (A_{—}23_P104116), APG4B (A_{—}23_P9903), CHCHD6 (A_{—}23_P91870), ATF6 (A_{—}23_P62907), OR52N5 (A_{—}23_P64474), (A_{—}23_P154442), LPP (A_{—}23_P251118), GPR62 (A_{—}23_P434289), PFKM (A_{—}23_P170848), MGC13379 (A_{—}23_P203389), ATRX (A_{—}23_P136874), KIAA1324 (A_{—}23_P115392), TP53BP2 (A_{—}23_P12523), ARL8 (A_{—}23_P161407), GRB2 (A_{—}23_P418537), PDCD2 (A_{—}23_P145146), (A_{—}23_P74368), ZNF224 (A_{—}23_P22286), (A_{—}23_P206032), DKFZp566C0424 (A_{—}23_P115106), (A_{—}23_P252796), (A_{—}23_P81780), GFRA3 (A_{—}23_P41992), KIF13B (A_{—}23_P95441), MGC40214 (A_{—}23_P330486), ITLN1 (A_{—}23_P84388), FXYD2 (A_{—}23_P161769), MGC5391 (A_{—}23_P5566), PABPC3 (A_{—}23_P48314), TRIM25 (A_{—}23_P15326), HIAT1 (A_{—}23_P45851), IFNA17 (A_{—}23_P169307), PMS1 (A_{—}23_P40063), MGC4645 (A_{—}23_P159071), LOC65121 (A_{—}23_P86072), DRD5 (A_{—}23_P374129), PTPRC (A_{—}23_P12392), ACTN1 (A_{—}23_P105957), LOC221143 (A_{—}23_P151368), ANKRD12 (A_{—}23_P130293), (A_{—}23_P43630), MAN2B1 (A_{—}23_P27613), E2F5 (A_{—}23_P31713), ROBO4 (A_{—}23_P344421), ZNF70 (A_{—}23_P154981), HIST1H1T (A_{—}23_P133842), B3GALT6 (A_{—}23_P35021), SCRN3 (A_{—}23_P17015), UCK2 (A_{—}23_P487), TAS2R43 (A_{—}23_P371254), SHMT2 (A_{—}23_P158239), OR4D1 (A_{—}23_P66392), PPP4R2 (A_{—}23_P41159), SLC25A1 (A_{—}23_P120773), TAF2 (A_{—}23_P43248), LY6G5C (A_{—}23_P145323), NPY5R (A_{—}23_P251836), WDR18 (A_{—}23_P5115), NOD3 (A_{—}23_P329399), TRIP12 (A_{—}23_P147984), CDC37 (A_{—}23_P130527), GLP2R (A_{—}23_P141309), HTR6 (A_{—}23_P74766), APG7L (A_{—}23_P143992), PQBP1 (A_{—}23_P62136), CAMLG (A_{—}23_P213728), WDSAM1 (A_{—}23_P108657), SLC35B3 (A_{—}23_P145463), FLJ20457 (A_{—}23_P216564), DOC2A (A_{—}23_P340953), KIAA1972 (A_{—}23_P3784), (A_{—}23_P52517), FLJ00012 (A_{—}23_P36305), LOC51252 (A_{—}23_P258992), (A_{—}23_P70077), TRAR1 (A_{—}23_P93531), IL17D (A_{—}23_P345692), NOG (A_{—}23_P341938), FLJ36749 (A_{—}23_P387624), OR2B2 (A_{—}23_P111088), PLD3 (A_{—}23_P27515), ADAMTSL3 (A_{—}23_P43940), TPCN1 (A_{—}23_P423208), TMEM30A (A_{—}23_P70290), MC4R (A_{—}23_P50121), FKSG83 (A_{—}23_P300080), KRTAP4-4 (A_{—}23_P38603), MPP5 (A_{—}23_P99536), (A_{—}23_P85874), BCAP29

(A_{—}23_P412526), PABPC1 (A_{—}23_P82693), CBR4 (A_{—}23_P213237), PTPDC1 (A_{—}23_P20876), CR12 (A_{—}23_P365844), FLJ20530 (A_{—}23_P43079), ALG8 (A_{—}23_P13554), CNOT6L (A_{—}23_P110304), KIF5B (A_{—}23_P86403), AGPAT5 (A_{—}23_P216200), CDCA7 (A_{—}23_P251421), FLJ14100 (A_{—}23_P121), IPO4 (A_{—}23_P162970), TMLHE (A_{—}23_P217546), ZNF527 (A_{—}23_P101932), OXA1L (A_{—}23_P2998), (A_{—}23_P118530), SIAT8D (A_{—}23_P41693), PTER (A_{—}23_P323774), T3JAM (A_{—}23_P201227), OR4A5 (A_{—}23_P98699), ZNF189 (A_{—}23_P216869), COPS4 (A_{—}23_P43779), NYD-SP21 (A_{—}23_P98561), Sep-10 (A_{—}23_P91168), IFNG (A_{—}23_P151294), TBR1 (A_{—}23_P5686), C20orf52 (A_{—}23_P143417), P15RS (A_{—}23_P55641), HMGA1 (A_{—}23_P42331), NARG1L (A_{—}23_P14204), POFUT2 (A_{—}23_P211227), (A_{—}23_P30509), CLEC1 (A_{—}23_P204669), (A_{—}23_P209251), PPP1R15A (A_{—}23_P90172), TADA2L (A_{—}23_P66664), LDHD (A_{—}23_P54918), NMUR2 (A_{—}23_P122134), LOC51035 (A_{—}23_P98605), GM2A (A_{—}23_P144872), (A_{—}23_P69931), WDR20 (A_{—}23_P205256), EXOSC4 (A_{—}23_P32463), ARHGEF7 (A_{—}23_P105845), SCEL (A_{—}23_P500000), (A_{—}23_P95619), BAG2 (A_{—}23_P255363), BACH1 (A_{—}23_P211047), OR8K3 (A_{—}23_P36050), CFL2 (A_{—}23_P65401), STK32B (A_{—}23_P21519), C7orf2 (A_{—}23_P300728), APRIN (A_{—}23_P205098), SLC2A12 (A_{—}23_P8279), TWSG1 (A_{—}23_P27256), TEX264 (A_{—}23_P41005), TATDN1 (A_{—}23_P254974), FAM11B (A_{—}23_P131176), PAK2 (A_{—}23_P106441), BIVM (A_{—}23_P105833)

(e) LOC132671 (A_{—}23_P407112), PTPN13 (A_{—}23_P18493), F2R (A_{—}23_P213562), PPIC (A_{—}23_P84018), FAM46A (A_{—}23_P70660), LHX6 (A_{—}23_P32175), TTC3 (A_{—}23_P120703), C5orf13 (A_{—}23_P92794), TPST1 (A_{—}23_P145965), FLJ23577 (A_{—}23_P252388), PGM1 (A_{—}23_P52031), SERTAD4 (A_{—}23_P318904), PXMP2 (A_{—}23_P135517), EPHA1 (A_{—}23_P157330), MYO5C (A_{—}23_P140434), DNER (A_{—}23_P362148), POLR2A (A_{—}23_P141235), IF (A_{—}23_P7212), KTAA0182 (A_{—}23_P152415), EDG2 (A_{—}23_P216609), ASRGL1 (A_{—}23_P203391), PLEKHE1 (A_{—}23_P89762), CROP (A_{—}23_P207493), ZFP36 (A_{—}23_P39237), DNAJC1 (A_{—}23_P127128), PXMP2 (A_{—}23_P124122), IPO7 (A_{—}23_P150714), TRA1 (A_{—}23_P2601), APOC3 (A_{—}23_P203183), TUBB2 (A_{—}23_P19291), SLC22A18 (A_{—}23_P139260), PLEKHK1 (A_{—}23_P52410), CD24 (A_{—}23_P114457), ZIC2 (A_{—}23_P36972), FTHL17 (A_{—}23_P148410), DUSP23 (A_{—}23_P103232), DPP7 (A_{—}23_P21574), AGXT2L1 (A_{—}23_P257231), ROR2 (A_{—}23_P158318), MAN2A1 (A_{—}23_P360769), HOXB7 (A_{—}23_P55276), FLJ20315 (A_{—}23_P3934), PPP1R14C (A_{—}23_P45011), CGI-115 (A_{—}23_P23356), EPLIN (A_{—}23_P151267), DIRC1 (A_{—}23_P131139), DPP3 (A_{—}23_P116091), CACNB2 (A_{—}23_P326852), SPDEF (A_{—}23_P111194), HDAC2 (A_{—}23_P122304), MSH3 (A_{—}23_P122001), EGR2 (A_{—}23_P46936), IL28RA (A_{—}23_P74112), SMPDL3A (A_{—}23_P72117), CAV1 (A_{—}23_P134454), LGALS3BP (A_{—}23_P15353), HCP5 (A_{—}23_P111125), OSBPL6 (A_{—}23_P108823), HOXB5 (A_{—}23_P363316), CLTB (A_{—}23_P43742), FLJ20054 (A_{—}23_P320897), KLF9 (A_{—}23_P415-401), STYK1 (A_{—}23_P13822), NR4Al (A_{—}23_P128230), TACSTD1 (A_{—}23_P91081), ZNF608 (A_{—}23_P169978), FTH1 (A_{—}23_P87199), YPEL3 (A_{—}23_P15104), TM4SF9 (A_{—}23_P61886), HSPH1 (A_{—}23_P88119), ARL4A (A_{—}23_P145760), SLC1A1 (A_{—}23_P216468), CELSR1 (A_{—}23_P132378), RICS (A_{—}23_P161686), ITM2B (A_{—}23_P139934), PDE9A (A_{—}23_P29096), SHOX2 (A_{—}23_P112974), JAG1 (A_{—}23_P210763), DCP1A (A_{—}23_P166826), RIF1 (A_{—}23_P160689), EIF3S6 (A_{—}23_P154526), CLTB (A_{—}23_P252677), NNT (A_{—}23_P70148), PRKAR2B (A_{—}23_P42975), ZNF467 (A_{—}23_P59470), ID3 (A_{—}23_P137381), SEMA3C (A_{—}23_P256473), MLF1 (A_{—}23_P143906), NMU (A_{—}23_P69537), CHN1 (A_{—}23_P79482), TPD52L1 (A_{—}23_P31143), LAD1 (A_{—}23_P415510), DSC3 (A_{—}23_P208029), GJB2 (A_{—}23_P204947), PPP3CA (A_{—}23_P92623), SE57-1 (A_{—}23_P363255), VSNL1 (A_{—}23_P209978), PLOD2 (A_{—}23_P211910), EPM2AIP1 (A_{—}23_P415622), SCOC (A_{—}23_P167291), PDE8B (A_{—}23_P259065), ID2 (A_{—}23_P143143), BMP4 (A_{—}23_P54140), (A_{—}23_P253896), FGF2 (A_{—}23_P218918), RDX (A_{—}23_P203027),

(f) NR2F2 (A_{—}23_P88589), (A_{—}23_P101121), LOC129293 (A_{—}23_P255897), EML1 (A_{—}23_P205746), CDKN2A (A_{—}23_P250888), NCR1 (A_{—}23_P108042), ZNF586 (A_{—}23_P107693), (A_{—}23_P75585), FKBP9 (A_{—}23_P334709), GUCA1B (A_{—}23_P81825), PTGER4 (A_{—}23_P148047), CREB1 (A_{—}23_P79231), TFRC (A_{—}23_P212617), TMSB10 (A_{—}23_P68102), KLF7 (A_{—}23_P67977), C14orf145 (A_{—}23_P430201), NAG8 (A_{—}23_P250097), IL27 (A_{—}23_P315320), AFP (A_{—}23_P58201), DHX57 (A_{—}23_P28307), FBXW10 (A_{—}23_P218358), CRYAA (A_{—}23_P306755), HPCA (A_{—}23_P126162), CHST12 (A_{—}23_P310421), LOC340529 (A_{—}23_P159897), C14orf159 (A_{—}23_P48771), DHRS2 (A_{—}23_P48570), NFE2 (A_{—}23_P13753), PIK3C2B (A_{—}23_P200710), PSORS1C1 (A_{—}23_P133900), RAPGEF2 (A_{—}23_P133095), KIAA1797 (A_{—}23_P21673), EIF4EBP2 (A_{—}23_P115922), KLF6 (A_{—}23_P63798), S100A4 (A_{—}23_P94800), (A_{—}23_P164368), ZBTB10 (A_{—}23_P385114), SPRR2A (A_{—}23_P148949), TFAP2C (A_{—}23_P120472), C1orf24 (A_{—}23_P421326), KIAA1333 (A_{—}23_P99604), PPP1R14c (A_{—}23_P257617), MUM1 (A_{—}23_P400217), BMP2 (A_{—}23_P143324), PAPLN (A_{—}23_P140347), ARL61P (A_{—}23_P118142), ARID3A (A_{—}23_P16516), FANCA (A_{—}23_P206441), KIAA0040 (A_{—}23_P51327), PPIC (A_{—}23_P422724), FLJ23311 (A_{—}23_P35871), DYM (A_{—}23_P170518), BSCL2 (A_{—}23_P150566), ARGBP2 (A_{—}23_P121795), DSC2 (A_{—}23_P4494), DPP7 (A_{—}23_P72830), EFNA1 (A_{—}23_P254512), MGC10911 (A_{—}23_P168637), MFGE8 (A_{—}23_P48951), DLAT (A_{—}23_P203030), KIAA1468 (A_{—}23_P356125), KIAA0907 (A_{—}23_P74458), C11orf8 (A_{—}23_P52888), FLJ10156 (A_{—}23_P49878), MGC4308 (A_{—}23_P359174), KIAA1598 (A_{—}23_P202587), RANBP2L1 (A_{—}23_P384090), (A_{—}23_P200843), HMGB1 (A_{—}23_P99985), ANKHD1 (A_{—}23_P58443), ZNF92 (A_{—}23_P501080), PIB5PA (A_{—}23_P91669), ZIC3 (A_{—}23_P327910), BTBD11 (A_{—}23_P419712), DJ971N18.2 (A_{—}23_P166100), CHES1 (A_{—}23_P140405), THBD (A_{—}23_P91390), ASGR2 (A_{—}23_P130116), FLJ10858 (A_{—}23_P155711), NDOR1 (A_{—}23_P257407), CD164 (A_{—}23_P254756), GLTSCR1 (A_{—}23_P130773), RRBP1 (A_{—}23_P120566), RANBP2L1 (A_{—}23_P218637), DKFZp762A217 (A_{—}23_P382705), MYO9A (A_{—}23_P205841), CASP7 (A_{—}23_P12572), GPRC5B (A_{—}23_P324327), PP2447 (A_{—}23_P166553), FLRT1 (A_{—}23_P47168), SFRP5 (A_{—}23_P1355), CBX3 (A_{—}23_P31315), EIF5B (A_{—}23_P218608), GRIN2D (A_{—}23_P153549), HNRPH1 (A_{—}23_P252991), PPAP2A (A_{—}23_P70060), LOC284361 (A_{—}23_P208674), USP53 (A_{—}23_P167338), BICD2 (A_{—}23_P157751), UBE2E2 (A_{—}23_P10807), GLCC11 (A_{—}23_P82402), CGI-143 (A_{—}23_P46507), FECH (A_{—}23_P89789), MBNL3 (A_{—}23_P96205), PTPRG (A_{—}23_P41054), FRMPD2 (A_{—}23_P86710), LCE1D (A_{—}23_P375524), RNF34 (A_{—}23_P128396), ITGB5 (A_{—}23_P166627), (A_{—}23_P206741), BIRC5 (A_{—}23_P118815), LMNB1 (A_{—}23_P258493), (A_{—}23_P44235), TM4SF5 (A_{—}23_P27107), —PTEN (A_{—}23_P98085), ACAS2L (A_{—}23_P120594), HNRPH2 (A_{—}23_P11283), CALM3 (A_{—}23_P4944), H2AFV (A_{—}23_P145904), RACGAP1 (A_{—}23_P65110), FLJ20160 (A_{—}23_P28530), MGC13204 (A_{—}23_P105583), VPS35 (A_{—}23_P66149), FLJ37078 (A_{—}23_P331700), PPARBP (A_{—}23_P425704), ASAM (A_{—}23_P35995), NPC1L1 (A_{—}23_P20075), SUCNR1 (A_{—}23_P69171), EPHA8 (A_{—}23_P103199), (A_{—}23_P16562), C14orf24 (A_{—}23_P2935), SLC30A3 (A_{—}23_P302568), SGEF (A_{—}23_P69100), MIDN (A_{—}23_P153709), RPN1 (A_{—}23_P132803), PSPC1 (A_{—}23_P76610), MGC39633 (A_{—}23_P92765), ZFOC1 (A_{—}23_P359654), MAT2A (A_{—}23_P401568), (A_{—}23_P353541), FREM1 (A_{—}23_P43334), ZNF449 (A_{—}23_P309865), KLHL7 (A_{—}23_P324994), SRRM1 (A_{—}23_P23803), GYPA (A_{—}23_P212854), FLJ21827 (A_{—}23_P116202), THEA (A_{—}23_P417-415), NICN1 (A_{—}23_P110005), UNQ1912 (A_{—}23_P30283), LRRC1 (A_{—}23_P215024), PLSCR4 (A_{—}23_P91912), PP1665 (A_{—}23_P87401), ELF2 (A_{—}23_P132948), AZI2 (A_{—}23_P109682), CBLB (A_{—}23_P212715), IFNK (A_{—}23_P310372), GNA14 (A_{—}23_P415561), (A_{—}23_P341418), TNRC6C (A_{—}23_P392501), MCM3 (A_{—}23_P7873), NDE1 (A_{—}23_P206901), RHBG (A_{—}23_P51690), CABYR (A_{—}23_P314712), MFN2 (A_{—}23_P126135), PHF13 (A_{—}23_P384559), SCD4 (A_{—}23_P213288), (A_{—}23_P167432), ET (A_{—}23_P26704), (A_{—}23_P65040), IQCC (A_{—}23_P74162), LCN7 (A_{—}23_P85585), TBC1D16 (A_{—}23_P428326), DNM1DN8-2 (A_{—}23_P106472), METRNL (A_{—}23_P10591), FBXO17 (A_{—}23_P101875), FTO (A_{—}23_P113184), NDUFS4 (A_{—}23_P257198), (A_{—}23_P259103), TRIM40 (A_{—}23_P402778), C20orf179 (A_{—}23_P330618), ACRBP (A_{—}23_P151120), UGT2B7 (A_{—}23_P136671), ZNF131 (A_{—}23_P133315), FLJ22353 (A_{—}23_P85853), DIAPH2 (A_{—}23_P113172), NOLC1 (A_{—}23_P314151), NUSAP1 (A_{—}23_P206183), HT017 (A_{—}23_P40856), MKL2 (A_{—}23_P54556), TRA2A (A_{—}23_P31386), PRC1 (A_{—}23_P206059), GOSR1 (A_{—}23_P252641), WIRE (A_{—}23_P366454), LMLN (A_{—}23_P43786), ART5 (A_{—}23_P427119), HLA-DQB2 (A_{—}23_P19510), ADK (A_{—}23_P112596), RNF26 (A_{—}23_P64630), COPG (A_{—}23_P44617), (A_{—}23_P26311), BAT2 (A_{—}23_P30870), (A_{—}23_P141263), DSCR4 (A_{—}23_P40455), RLBP1 (A_{—}23_P163361), TFCP2 (A_{—}23_P347528), AEGP (A_{—}23_P71787), ZNF365 (A_{—}23_P86610), PHOX2B (A_{—}23_P213228), ARPP-19 (A_{—}23_P205768), WNT10B (A_{—}23_P162317), AGRP (A_{—}23_P502320), PPP1R9B (A_{—}23_P55242), P4HA1 (A_{—}23_P35521), HDAC1 (A_{—}23_P114656), CENTA2 (A_{—}23_P49816), PIN4 (A_{—}23_P315345), (A_{—}23_P72949), CHSY1 (A_{—}23_P37484), FTSJ2 (A_{—}23_P31253), RFP (A_{—}23_P42224), TAPBPL (A_{—}23_P36698), ADSSL1 (A_{—}23_P76823), FLJ31153 (A_{—}23_P390744), LRP1B (A_{—}23_P5342), BRD1 (A_{—}23_P166536), QTRTD1 (A_{—}23_P91930), (A_{—}23_P18023), FLJ25555 (A_{—}23_P380881), (A_{—}23_P70998), SYNJ2 (A_{—}23_P316974), (A_{—}23_P167624), ALS2CR3 (A_{—}23_P209426), FLJ34443 (A_{—}23_P431330), SOX4 (A_{—}23_P82176), VDR (A_{—}23_P162589), FBXL2 (A_{—}23_P17955), D4S234E (A_{—}23_P371106), CXorf6 (A_{—}23_P251075), KIF24 (A_{—}23_P43595), ETV7 (A_{—}23_P42347), FLJ14721 (A_{—}23_P128375), MGC13168 (A_{—}23_P150960), CAP350 (A_{—}23_P201816), BCAS1 (A_{—}23_P17420), IL4R (A_{—}23_P129556), CTAG2 (A_{—}23_P22693), CDV1 (A_{—}23_P72680), RBM15 (A_{—}23_P34879), MGC34923 (A_{—}23_P91850), ING2 (A_{—}23_P125687), BTBD11 (A_{—}23_P99349), SUV420H1 (A_{—}23_P217968), (A_{—}23_P158925), CTSD (A_{—}23_P52556), C1QTNF3 (A_{—}23_P122068), MTA1 (A_{—}23_P158129), TNK2 (A_{—}23_P259846), C21orf81 (A_{—}23_P392529), MTCP1 (A_{—}23_P11295), TDRD3 (A_{—}23_P2711), (A_{—}23_P253774), (A_{—}23_P139725), USP10 (A_{—}23_P100196), BCL3 (A_{—}23_P4662), GALNS (A_{—}23_P106562), DIRAS2 (A_{—}23_P379778), KIAA0467 (A_{—}23_P51639), RHOU (A_{—}23_P114814), (A_{—}23_P50297), (A_{—}23_P252175), FLJ37099 (A_{—}23_P46315), MFHAS1 (A_{—}23_P112078), GAGED4 (A_{—}23_P114343), PIK3CB (A_{—}23_P92253), ECT2 (A_{—}23_P9571), MAP3K71P1 (A_{—}23_P80345), (A_{—}23_P110557), ASB12 (A_{—}23_P96395), C1QTNF6 (A_{—}23_P57513), FGF12 (A_{—}23_P169553), CNTN3 (A_{—}23_P250173), LOC150678 (A_{—}23_P377212), SUPT3H (A_{—}23_P251621), C19orf14 (A_{—}23_P339705), PRKCZ (A_{—}23_P51187), SEMA5A (A_{—}23_P213415), TRIM6 (A_{—}23_P33675), CIDEA (A_{—}23_P258592), ATR (A_{—}23_P124664), HIST1H2AC (A_{—}23_P372860), DI02 (A_{—}23_P48740), FBXL18 (A_{—}23_P423957), DKFZP434HO115 (A_{—}23_P73150), CTSO (A_{—}23_P110175),

(g) CYorf15B (A_{—}23_P96652), MTUS1 (A_{—}23_P94358), RBPMS2 (A_{—}23_P100059), DKK1 (A_{—}23_P24129), KCNMB4 (A_{—}23_P64792), NALP2 (A_{—}23_P130824), EMILIN2 (A_{—}23_P27315), (A_{—}23_P14216), PRKG1 (A_{—}23_P136041), SERPINE2 (A_{—}23_P50919), FOXA1 (A_{—}23_P37127), SPINT2 (A_{—}23_P27795), PTPRK (A_{—}23_P254924), HSPC105 (A_{—}23_P129458), MCTP2 (A_{—}23_P65789), KBTBD7 (A_{—}23_P25605), DKFZp762K222 (A_{—}23_P251364), TBX1 (A_{—}23_P211345), COBLL1 (A_{—}23_P79289), DSP (A_{—}23_P31031), CCL28 (A_{—}23_P503072), TCF7L2 (A_{—}23_P127014), C3orf4 (A_{—}23_P155556), CTHRC1 (A_{—}23_P111888), ATP6V1C2 (A_{—}23_P250914), MTAC2D1 (A_{—}23_P413075), RBM24 (A_{—}23_P167812), FLJ20972 (A_{—}23_P366890), CLOCK (A_{—}23_P419038), KLRC3 (A_{—}23_P22232), MGC11242 (A_{—}23_P118894), HMMR (A_{—}23_P70007), FBLN5 (A_{—}23_P151805), SPINK2 (A_{—}23_P155688), RRAGD (A_{—}23_P133684), CCL2 (A_{—}23_P89431), PSCD1 (A_{—}23_P95184), PDZRN3 (A_{—}23_P21618), MYOM2 (A_{—}23_P258912), UGP2 (A_{—}23_P253046), IFITM1 (A_{—}23_P72737), PSTPIP2 (A_{—}23_P208119), CDH3 (A_{—}23_P49155), SASH1 (A_{—}23_P93442), HOXB6 (A_{—}23_P66682), PITX1 (A_{—}23_P58636), CYP27B1 (A_{—}23_P36397), STXBP6 (A_{—}23_P205713), PCK1 (A_{—}23_P408249), MYB (A_{—}23_P31073), DKFZP434P1750 (A_{—}23_P22382), TCF₇L2 (A_{—}23_P328501), GALNT13 (A_{—}23_P165369), ME1 (A_{—}23_P8196), FZD3 (A_{—}23_P347471), ADD3 (A_{—}23_P202435), FLJ13391 (A_{—}23_P108676), TGFBR3 (A_{—}23_P200780), CYB5 (A_{—}23_P101208), (A_{—}23_P58697), KIAA0882 (A_{—}23_P41487), DNCLI2 (A_{—}23_P9688), GSDML (A_{—}23_P66454), SYTL2 (A_{—}23_P53193), PHGDH (A_{—}23_P85780), FZD7 (A_{—}23_P209449), GSTP1 (A_{—}23_P202658), HEYL (A_{—}23_P46245), BSN (A_{—}23_P29735), PIR (A_{—}23_P137035), CNTNAP2 (A_{—}23_P95802), C2orf23 (A_{—}23_P96285), IFITM3 (A_{—}23_P87539), 13CDNA73 (A_{—}23_P105862), UGDH (A_{—}23_P167067), HOXB8 (A_{—}23_P370586), LRP11 (A_{—}23_P19652), DLX4 (A_{—}23_P164196), SYNCRIP (A_{—}23_P214798), NCOA1 (A_{—}23_P39594), CD9 (A_{—}23_P76364), C6orf60 (A_{—}23_P167818), FGFR3 (A_{—}23_P500501), (A_{—}23_P35546), ERBB3 (A_{—}23_P349416), MAP3K5 (A_{—}23_P134125), IFRG28 (A_{—}23_P166797), SGCE (A_{—}23_P254626), LOC148418 (A_{—}23_P61438), GSTA1 (A_{—}23_P135417), MTCH1 (A_{—}23_P145388), OVOL1 (A_{—}23_P202810), PAH (A_{—}23_P2502), RBP7 (A_{—}23_P97431), ASS (A_{—}23_P31922), CAMTA2 (A_{—}23_P365189), FLJ30525 (A_{—}23_P309361), TM4SF9 (A_{—}23_P323930), EFHD1 (A_{—}23_P154338), CDH1 (A_{—}23_P206359), (A_{—}23_P138635), LPL (A_{—}23_P146233), RUNX3 (A_{—}23_P51231), PAG (A_{—}23_P347070), SLC40A1 (A_{—}23_P102391), SLC43A3 (A_{—}23_P358545), SLC12A2 (A_{—}23_P133606), MTAC2D1 (A_{—}23_P88439), CLDN4 (A_{—}23_P19944), MAP3K12 (A_{—}23_P105405), EPHB6 (A_{—}23_P145935), PERP (A_{—}23_P214950), ID1 (A_{—}23_P252306), MID1 (A_{—}23_P32838), FLJ10901 (A_{—}23_P1043), STX6 (A_{—}23_P97725), GABARAPL1 (A_{—}23_P65817), PAPSS2 (A_{—}23_P104492), PERP (A_{—}23_P369298)_UBE1L (A_{—}23_P9809), EDG8 (A_{—}23_P107744), TGFB1 (A_{—}23_P412335), MIDI (A_{—}23_P170037), SMAD7 (A_{—}23_P55518), HYAL3 (A_{—}23_P212400), PRRX2 (A_{—}23_P83298), KITLG (A_{—}23_P204654), CPOX (A_{—}23_P144179), CARD15 (A_{—}23_P420863), AXIN2 (A_{—}23_P148014), ICK (A_{—}23_P214315), AXIN2 (A_{—}23_P159395), LIN7C (A_{—}23_P139277), GNAI1 (A_{—}23_P122976), LGALS3 (A_{—}23_P128918), ZFP36L2 (A_{—}23_P101960), LHX2 (A_{—}23_P32165), RECK (A_{—}23_P83028), CKB (A_{—}23_P25674), C6orf114 (A_{—}23_P134058), SNTB1 (A_{—}23_P95029), KLF5 (A_{—}23_P53891), DPP4 (A_{—}23_P39885), LOC146177 (A_{—}23_P129397), LAPTM4B (A_{—}23_P59926), (A_{—}23_P41664), PROM1 (A_{—}23_P258462), MPP1 (A_{—}23_P171296), KCNS3 (A_{—}23_P120105), PDGFC (A_{—}23_P58396), HEPH (A_{—}23_P22526), CHAT (A_{—}23_P46894), GAMT (A_{—}23_P108143), ELOVL5 (A_{—}23_P156498), KLHL13 (A_{—}23_P159974), MIDI (A_{—}23_P10031), FLJ11196 (A_{—}23_P117782), MAN1A1 (A_{—}23_P156431), MAOA (A_{—}23_P96410), DDX3Y (A_{—}23_P217797), SORL1 (A_{—}23_P87049), C1QL3 (A_{—}23_P1186), MTERF (A_{—}23_P111712),

(h) PLCE1 (A_{—}23_P35617), PTPRK (A_{—}23_P254242), KLRC1 (A_{—}23_P151046), PTPN12 (A_{—}23_P8763), ATP1B1 (A_{—}23_P62932), CGREF1 (A_{—}23_P210235), RHBDL6 (A_{—}23_P329870), BARD1 (A_{—}23_P67771), (A_{—}23_P61112), C14orf101 (A_{—}23_P205607), ZNF205 (A_{—}23_P3748), VPREB1 (A_{—}23_P29152), KIF12 (A_{—}23_P60550), CXCR4 (A_{—}23_P102000), CHST7 (A_{—}23_P319617), TARDBP (A_{—}23_P403955), CLDN23 (A_{—}23_P134854), MED12 (A_{—}23_P73699), IRF2BP2 (A_{—}23_P46588), CYP26A1 (A_{—}23_P138655), TJP2 (A_{—}23_P9293), SIPA1L2 (A_{—}23_P137470), GDF8 (A_{—}23_P165727), GPSM2 (A_{—}23_P63402), AES (A_{—}23_P165061), SLC6A14 (A_{—}23_P171038), FNBP1 (A_{—}23_P32249), KIAA0998 (A_{—}23_P151756), SQRDL (A_{—}23_P3221), PCDH7 (A_{—}23_P212888), PTMA (A_{—}23_P434305), NOLC1 (A_{—}23_P202140), CXXC4 (A_{—}23_P121676), IPO7 (A_{—}23_P331598), TTC13 (A_{—}23_P103864), THBS1 (A_{—}23_P206212), MIDN (A_{—}23_P324461), RDHE2 (A_{—}23_P257457), USP43 (A_{—}23_P152696), BCLP (A_{—}23_P62768), FLJ33996 (A_{—}23_P399880), CDKN3 (A_{—}23_P48669), SEC31L2 (A_{—}23_P35564), HIST1H3F (A_{—}23_P30796), PLEKHA6 (A_{—}23_P431268), ENC1 (A_{—}23_P213424), POR (A_{—}23_P19964), DPYSL5 (A_{—}23_P210224), MLH1 (A_{—}23_P69058), HIST1H2AK (A_{—}23_P42220), B7-H4 (A_{—}23_P518), DYRK4 (A_{—}23_P87899), GAB1 (A_{—}23_P335239), IGSF9 (A_{—}23_P85441), DPYSL3 (A_{—}23_P7528), GALNT12 (A_{—}23_P415652), C10orf78 (A_{—}23_P413193), CDW92 (A_{—}23_P216630), ACVR1C (A_{—}23_P67820), GABARAPL1 (A_{—}23_P162640), EGFL9 (A_{—}23_P133786), CCR2 (A_{—}23_P212354), MYOD1 (A_{—}23_P53033), CR2 (A_{—}23_P124542), DAZAP2 (A_{—}23_P40025), C6orf85 (A_{—}23_P7882), ERP70 (A_{—}23_P42800), DST (A_{—}23_P59388), SLC35E2 (A_{—}23_P61860), FZD3 (A_{—}23_P215922), SMCY (A_{—}23_P137238), MMP11 (A_{—}23_P57417), LCE1C (A_{—}23_P114607), MESDC1 (A_{—}23_P99891), (A_{—}23_P20168), HSD17B4 (A_{—}23_P92954), BDKRB2 (A_{—}23_P304897), GALNT12 (A_{—}23_P257731), ARK5 (A_{—}23_P348257), LUZP4 (A_{—}23_P96611), SYP (A_{—}23_P136935), LHFP (A_{—}23_P88069), CSTB (A_{—}23_P154889), NR2F2 (A_{—}23_P422703), CULS (A_{—}23_P203009), CLDN1 (A_{—}23_P57779), KIF20A (A_{—}23_P256956), UQCRC2 (A_{—}23_P118002), NDUFA9 (A_{—}23_P76499), GALNT1 (A_{—}23_P306105), DHRS1 (A_{—}23_P48747), MOSPD1 (A_{—}23_P73828), PMM2 (A_{—}23_P432360), EIF2AK1 (A_{—}23_P251173), LNX (A_{—}23_P213139), LTBP1 (A_{—}23_P43810), TM4SF2 (A_{—}23_P114185), CHST11 (A_{—}23_P139919), TIMP3 (A_{—}23_P211468), BMP7 (A_{—}23_P68488), (A_{—}23_P34007), HRIHFB2122 (A_{—}23_P17854), ENPEP (A_{—}23_P144596), C1S (A_{—}23_P2492), IDH2 (A_{—}23_P129204), RNASE4 (A_{—}23_P428738), SLC17A4 (A_{—}23_P357270), NRAP (A_{—}23_P402765), BTG3 (A_{—}23_P80068), MGC23401 (A_{—}23_P150950), C1orf34 (A_{—}23_P160214), ATP5G3 (A_{—}23_P56678), LOC92305 (A_{—}23_P354175), C6orf111 (A_{—}23_P122617), TBC1D8 (A_{—}23_P253281), MTMR8 (A_{—}23_P84995), (A_{—}23_P310900), UBA2 (A_{—}23_P209020), STK33 (A_{—}23_P127915), BCHE (A_{—}23_P212050), OGFR (A_{—}23_P28707), CITED2 (A_{—}23_P214969), KIAA1961 (A_{—}23_P144994), FLJ10707 (A_{—}23_P212204), (A_{—}23_P93311), GOSR1 (A_{—}23_P4373), PMAIP1 (A_{—}23_P207999), ADAM20 (A_{—}23_P48698), LAMB2 (A_{—}23_P21382), GTDC1 (A_{—}23_P153945), PDXK (A_{—}23_P68730), SGK (A_{—}23_P19673), LOC90637 (A_{—}23_P336992), SRP72 (A_{—}23_P337201), RAB31 (A_{—}23_P141688), SLC9A3R1 (A_{—}23_P152593), CTBP2 (A_{—}23_P63897), DLGAP3 (A_{—}23_P149707), SLC9A3R1 (A_{—}23_P308519), FLJ38973 (A_{—}23_P142916), RHOBTB1 (A_{—}23_P35634), ZCCHC2 (A_{—}23_P66958), KIAA1036 (A_{—}23_P76998), CTSL2 (A_{—}23_P146456), (A_{—}23_P111797), DBC1 (A_{—}23_P94517), MTMR1 (A_{—}23_P73530), C9orf55 (A_{—}23_P157726), SERPINF1 (A_{—}23_P100660), SH3KBP1 (A_{—}23_P374777), C9orf19 (A_{—}23_P71627), ANGPTL1 (A_{—}23_P126706), (A_{—}23_P64184), FKBP1B (A_{—}23_P142631), VAMP8 (A_{—}23_P28434), HOXD3 (A_{—}23_P79652), MBIP (A_{—}23_P2922), CD48 (A_{—}23_P74145), RHBDL1 (A_{—}23_P26468), MCC (A_{—}23_P377114), PMM2 (A_{—}23_P206937), FGD1 (A_{—}23_P73559), EIF4G2 (A_{—}23_P104892), TRADD (A_{—}23_P54649), CGN (A_{—}23_P149388), HCAP-G (A_{—}23_P155815), SMARCC2 (A_{—}23_P128073), CAPS2 (A_{—}23_P124773), NHLRC2 (A_{—}23_P46767), PC (A_{—}23_P161647), VRK2 (A_{—}23_P119992), OR3A1 (A_{—}23_P50031), GRP58 (A_{—}23_P99883), FXR1 (A_{—}23_P132784), MMRN2 (A_{—}23_P150057), PON1 (A_{—}23_P168598), MCMDC1 (A_{—}23_P397347), ZA20D2 (A_{—}23_P71752), KIRREL2 (A_{—}23_P16583), SFRS2 (A_{—}23_P77776), MRPS7 (A_{—}23_P3973), HS6ST1 (A_{—}23_P90579), (A_{—}23_P258144), ZF (A_{—}23_P203649), MARCH-IX (A_{—}23_P47777), PPARα (A_{—}23_P40724), SESN3 (A_{—}23_P361448), KPNA2 (A_{—}23_P125265), RGC32 (A_{—}23_P204937), SYT1 (A_{—}23_P36795), (A_{—}23_P150996), SS18 (A_{—}23_P141738), ITIH2 (A_{—}23_P202053), C7orf24 (A_{—}23_P42695), (A_{—}23_P169828), SP100 (A_{—}23_P349928), LOC90379 (A_{—}23_P50399), CAMK4 (A_{—}23_P250347), USP52 (A_{—}23_P64689), PTMA (A_{—}23_P210283), TUBB4Q (A_{—}23_P140884), (A_{—}23_P166280), KIAA1536 (A_{—}23_P98995), LRP2 (A_{—}23_P28295), A4GALT (A_{—}23_P57568), C13orf11 (A_{—}23_P205188), HIBADH (A_{—}23_P168507), (A_{—}23_P15226), USP8 (A_{—}23_P129053), VPS41 (A_{—}23_P215318), PCP4 (A_{—}23_P109322), PJA2 (A_{—}23_P133470), HMGB1 (A_{—}23_P162805), NOS3 (A_{—}23_P70849), ROCK2 (A_{—}23_P209689), NEBL (A_{—}23_P104522), ADORA1 (A_{—}23_P74299), HEXA (A_{—}23_P129096), ZNF573 (A_{—}23_P339079), ABCA5 (A_{—}23_P78018), (A_{—}23_P104781), NXN (A_{—}23_P61778), FLJ14166 (A_{—}23_P7684), RYR3 (A_{—}23_P94838), NJMU-R1 (A_{—}23_P15639), FLJ13213 (A_{—}23_P26094), ING1 (A_{—}23_P99437), C2orf15 (A_{—}23_P415511), NEXN (A_{—}23_P200001), TBC1D3 (A_{—}23_P78068), HLCS (A_{—}23_P304991), (A_{—}23_P256329), SAFB2 (A_{—}23_P27894), CACNB3 (A_{—}23_P204019), CNTN1 (A_{—}23_P204541), CRLF1 (A_{—}23_P56197), CPNE4 (A_{—}23_P327551), COPZ1 (A_{—}23_P116802), Sep-09 (A_{—}23_P106973), PDGFRL (A_{—}23_P60148), C14orf129 (A_{—}23_P205336), RALA (A_{—}23_P215302), LOC90799 (A_{—}23_P307400), TMEM29 (A_{—}23_P148519), FLJ10378 (A_{—}23_P7253), HOXB9 (A_{—}23_P27013), MARK1 (A_{—}23_P424), CSNK1G1 (A_{—}23_P83704), FLJ20674 (A_{—}23_P72058), MPHOSPH1 (A_{—}23_P75071), SMARCA3 (A_{—}23_P57676), PROCA1 (A_{—}23_P427148), DHRS8 (A_{—}23_P408271), FANCL (A_{—}23_P131383), DHRSX (A_{—}23_P251339), STXBP2 (A_{—}23_P130614), ACO2 (A_{—}23_P103149), FLJ33069 (A_{—}23_P321351), FGFRL1 (A_{—}23_P92349), GALNT1 (A_{—}23_P153137), ADH1B (A_{—}23_P213184), EPSTI1 (A_{—}23_P105794), PCNT2 (A_{—}23_P57347), RNF165 (A_{—}23_P371280), HBZ (A_{—}23_P3651), LRRC4 (A_{—}23_P8625), OR5M9 (A_{—}23_P104939), PLGL (A_{—}23_P359630), GEFT (A_{—}23_P98844), PRRX1 (A_{—}23_P502731), EZH2 (A_{—}23_P259641), CHD3 (A_{—}23_P21640), SERPINA10 (A_{—}23_P128759), CGI-111 (A_{—}23_P250982), KIAA1447 (A_{—}23_P49539), FLJ13231 (A_{—}23_P213877), (A_{—}23_P146744), C1orf38 (A_{—}23_P873), ETVS (A_{—}23_P9831), SUCLA2 (A_{—}23_P117157), RNF44 (A_{—}23_P213592), PPM1D (A_{—}23_P89349), HDHD2 (A_{—}23_P153098), ITIH3 (A_{—}23_P6821), PCP2 (A_{—}23_P355860), C6orf210 (A_{—}23_P134167), C22orf24 (A_{—}23_P166431), FNBP1L (A_{—}23_P417942), TYMS (A_{—}23_P50096), ZMPSTE24 (A_{—}23_P137427), BTNL2 (A_{—}23_P376686), PLVAP (A_{—}23_P56328), DMXL1 (A_{—}23_P250571), CHES1 (A_{—}23_P88434), SRGAP1 (A_{—}23_P162446), RPIP8 (A_{—}23_P66579), ATP2B1 (A_{—}23_P128319), FCGR2B (A_{—}23_P34650), RCN1 (A_{—}23_P203299), FLJ10330 (A_{—}23_P201565), TNFAIP3 (A_{—}23_P156898), FLJ23191 (A_{—}23_P110266), GLG1 (A_{—}23_P206510), CCDC7 (A_{—}23_P374294), NBEA (A_{—}23_P21128), PB1 (A_{—}23_P218863), PAG (A_{—}23_P215841), ENPEP (A_{—}23_P333605), FLJ14281 (A_{—}23_P213199), CTAGE5 (A_{—}23_P128773), FLJ23467 (A_{—}23_P46355), IL18R1 (A_{—}23_P39735), RBM19 (A_{—}23_P204119), IK (A_{—}23_P133245), SC5□L (A_{—}23_P372888), LOC115509 (A_{—}23_P129659), CYYR1 (A_{—}23_P17620), ZNF589 (A_{—}23_P110031), ELOVL2 (A_{—}23_P251606), KIF23 (A_{—}23_P48835), PDE6A (A_{—}23_P81588), NDP52 (A_{—}23_P4221), IRX3 (A_{—}23_P152237), COX7C (A_{—}23_P110811), APEG1 (A_{—}23_P338919), CHRDL2 (A_{—}23_P127990), MYST3 (A_{—}23_P407628), C11orf15 (A_{—}23_P162087), HMGB3 (A_{—}23_P217236), ANKRD25 (A_{—}23_P50426), (A_{—}23_P218523), BAZ1B (A_{—}23_P215449), FLJ23033 (A_{—}23_P97481), (A_{—}23_P61937), ASK (A_{—}23_P254612), AK5 (A_{—}23_P200015), LOC147650 (A_{—}23_P27392), C10orf3 (A_{—}23_P115872), FSTL3 (A_{—}23_P209160), ARMET (A_{—}23_P132793), C18orf22 (A_{—}23_P153086), C6orf62 (A_{—}23_P422222), MYH2 (A_{—}23_P38271), SEC61G (A_{—}23_P71241), ZNF31 (A_{—}23_P23102), H2AFY (A_{—}23_P70045), MESDC2 (A_{—}23_P88503), MBNL1 (A_{—}23_P357811), HCN3 (A_{—}23_P34827), (A_{—}23_P203228), (A_{—}23_P125602), DERP6 (A_{—}23_P164289), LUC7L2 (A_{—}23_P59790), MUC15 (A_{—}23_P24332), NDUFV1 (A_{—}23_P127353), YWHAH (A_{—}23_P103070), RNF103 (A_{—}23_P56709), MAN2A2 (A_{—}23_P37564), (A_{—}23_P41340), CLDN11 (A_{—}23_P29800), RNF133 (A_{—}23_P42963), UBASH3A (A_{—}23_P6293), PB1 (A_{—}23_P365874), CDK5RAP3 (A_{—}23_P66900), Sep-03 (A_{—}23_P211572), (A_{—}23_P71179), CALML5 (A_{—}23_P43841), NEK2 (A_{—}23_P35219), EME1 (A_{—}23_P368225), USMG5 (A_{—}23_P127095), BM039 (A_{—}23_P88740), LBX1 (A_{—}23_P86493), GNG4 (A_{—}23_P335329), (A_{—}23_P45712), DLG7 (A_{—}23_P88331), GPRC5C (A_{—}23_P38167), APOH (A_{—}23_P38244), APBB3A_{—}23_P110445), ATP6VOA2 (A_{—}23_P87513), (A_{—}23_P30055), LCE3E (A_{—}23_P340340), FLJ23506 (A_{—}23_P50217), CXXC5 (A_{—}23_P213680), RAB10 (A_{—}23_P165879), HOZFP (A_{—}23_P407090), RABGAP1L (A_{—}23_P377264), MARCO (A_{—}23_P101992), OIP5 (A_{—}23_P379614), PELP1 (A_{—}23_P49898), PPY (A_{—}23_P207336), EFNB2 (A_{—}23_P428139), PPIA (A_{—}23_P258340), C20orf100 (A_{—}23_P154566), POPDC3 (A_{—}23_P358599), PPFIA1 (A_{—}23_P75509), ZNF652 (A_{—}23_P55256), MGC10334 (A_{—}23_P63281), SC5DL (A_{—}23_P98446), OVOL1 (A_{—}23_P326700), THOC2 (A_{—}23_P11051), DKFZP566D1346 (A_{—}23_P114616), MOCS1 (A_{—}23_P58993), RAD17 (A_{—}23_P159053), MRPL48 (A_{—}23_P162106), (A_{—}23_P54406), SP110 (A_{—}23_P120002), DTX3L (A_{—}23_P347040), MYLC2PL (A_{—}23_P393015), DMPK (A_{—}23_P50535), KIAA1279 (A_{—}23_P380815), FLJ23754 (A_{—}23_P365117), DGKD (A_{—}23_P210253), GCLM (A_{—}23_P103996), SLC1A7 (A_{—}23_P325562), RBBP8 (A_{—}23_P252371), KCNJ14 (A_{—}23_P130764), PDPR (A_{—}23_P206677), FRMD1 (A_{—}23_P81717), NUCB2 (A_{—}23_P13364), SS18L2 (A_{—}23_P166698), FLJ31713 (A_{—}23_P354326), TDRD4 (A_{—}23_P99373), S100A8 (A_{—}23_P200288), (A_{—}23_P42514), APOL4 (A_{—}23_P211479), HECTD2 (A_{—}23_P355525), (A_{—}23_P133257), ZCCHC11 (A_{—}23_P34433), LMAN2 (A_{—}23_P169599), (A_{—}23_P131036), KIAA0372 (A_{—}23_P61854), OSBPL5 (A_{—}23_P53081), CBX1 (A_{—}23_P107509), MOGAT2 (A_{—}23_P203692), FASN (A_{—}23_P44132), DKFZp762E1312 (A_{—}23_P79429), PTPRA A_{—}23_P28869), RYR2 (A_{—}23_P137797), PDPK1 (A_{—}23_P66219), DAPP1 (A_{—}23_P255444), TNRC6A (A_{—}23_P349310), KRAS2 (A_{—}23_P45140), TRIM15 (A_{—}23_P214554), RCOR2 (A_{—}23_P24397), C2orf22 (A_{—}23_P131375), TFDP1 (A_{—}23_P14243), RFC5 (A_{—}23_P95302), STAT1 (A_{—}23_P56630), MYBPH (A_{—}23_P148737), PCCA (A_{—}23_P48358), PSG9 (A_{—}23_P39309), ASPM (A_{—}23_P52017), (A_{—}23_P109676), DEGS2 (A_{—}23_P88450), FLJ32112 (A_{—}23_P167738), FLJ25179 (A_{—}23_P372962), GP1BB (A_{—}23_P29124), PRPF39 (A_{—}23_P163107), COL4A1 (A_{—}23_P65240), C1QTNF2 (A_{—}23_P92899), TFCP2L2 (A_{—}23_P5882), MTA1 (A_{—}23_P9513), PHF20 (A_{—}23_P428835), FLJ23342 (A_{—}23_P64280), R3HDM (A_{—}23_P380998), REC8L1 (A_{—}23_P322550), ACCN4 (A_{—}23_P56508), AIM1L (A_{—}23_P360324), SLCO1B1 (A_{—}23_P128254), (A_{—}23_P135837), TAC3 (A_{—}23_P2283), (A_{—}23_P73820), KLRC4 (A_{—}23_P218058), EP400 (A_{—}23_P253154), FLJ25955 (A_{—}23_P28466), GIP (A_{—}23_P141459), PSMA3 (A_{—}23_P140301), PPP2R5E (A_{—}23_P3042), P53AIP1 (A_{—}23_P340171), FLJ40873 (A_{—}23_P367014), PTPRJ (A_{—}23_P405048), FLJ39739 (A_{—}23_P74993), MAD1L1 (A_{—}23_P42657), C9orf39 (A_{—}23_P157963), EIF2B2 (A_{—}23_P25926), PIP5K2B (A_{—}23_P54983), MAML1 (A_{—}23_P110604), NRN1 (A_{—}23_P82088), Cep164 (A_{—}23_P75609), FLRT3 (A_{—}23_P166109), PAI-RBP1 (A_{—}23_P200661), C13orf6 (A_{—}23_P205027), ARID4B (A_{—}23_P201951), MKI67 (A_{—}23_P202232), LOC388152 (A_{—}23_P129103), UNG (A_{—}23_P76388), (A_{—}23_P63644), C1GALT1 (A_{—}23_P252145), RAB2 (A_{—}23_P253949), SDF2L1 (A_{—}23_P6344), GHR (A_{—}23_P156087), IGLC2 (A_{—}23_P72271), C21orf2 (A_{—}23_P211167), LY6G6C (A_{—}23_P8083), (A_{—}23_P20573), HSPA2 (A_{—}23_P88303), NPB (A_{—}23_P38545), C21orf91 (A_{—}23_P211015), KIAA0355 (A_{—}23_P324490), SLC17A6 (A_{—}23_P24294), BDKRB2 (A_{—}23_P140142), CDC5L (A_{—}23_P156471), ATF71P2 (A_{—}23_P129466), ABCB10 (A_{—}23_P201918), NOSTRIN (A_{—}23_P79360), FLJ11000 (A_{—}23_P31176), LMTK2 (A_{—}23_P410746), LOC124773 (A_{—}23_P164100), (A_{—}23_P255091), SP192 (A_{—}23_P593), ZFR (A_{—}23_P41818), OR51E2 (A_{—}23_P139327), TP53TG3 (A_{—}23_P37994), EIF2B1 (A_{—}23_P105313), PTPRCAP (A_{—}23_P98173), LIMR (A_{—}23_P150931), ELAC1 (A_{—}23_P15942), YWHAG (A_{—}23_P123022), (A_{—}23_P42565), ADAM11 (A_{—}23_P502158), LYZ (A_{—}23_P76192), ADK (A_{—}23_P125333), MSCP (A_{—}23_P216004), OR6T1 (A_{—}23_P161815), TFDP3 (A_{—}23_P10513), (A_{—}23_P159693), PRSS21 (A_{—}23_P129602), RTP1 (A_{—}23_P155407), COX411 (A_{—}23_P141028), RPS6KA2 (A_{—}23_P335920), GGA2 (A_{—}23_P163732), C14orf58 (A_{—}23_P140364), IFNAR2 (A_{—}23_P211083), NPDO14 (A_{—}23_P34396), FLJ10154 (A_{—}23_P162776), ATP9A (A_{—}23_P102664), NKX2-8 (A_{—}23_P341547), OR8U1 (A_{—}23_P13244), KIAA1128 (A_{—}23_P404108), CCR1 (A_{—}23_P6849), FLJ10916 (A_{—}23_P259201), BAGE (A_{—}23_P388331), CDC6 (A_{—}23_P49972), THOC4 (A_{—}23_P152984), (A_{—}23_P104970), NCKIPSD (A_{—}23_P29634), MYOC (A_{—}23_P23783), (A_{—}23_P157835), (A_{—}23_P46649), PARP1 (A_{—}23_P114783), ZNF278 (A_{—}23_P211459), (A_{—}23_P51082), UNQ3045 (A_{—}23_P122600), RPL4 (A_{—}23_P58031), MYBL2 (A_{—}23_P143184), TM4SF6 (A_{—}23_P171143), KCNC3 (A_{—}23_P101516), C21orf105 (A_{—}23_P154915), LOC220070 (A_{—}23_P388932), DUSP22 (A_{—}23_P120252), MGC16385 (A_{—}23_P343843), APG10L (A_{—}23_P92824), ITSN2 (A_{—}23_P28201), MGC14816 (A_{—}23_P330581), UGT2B28 (A_{—}23_P212968), C13orf7 (A_{—}23_P25638), PPARGC1B (A_{—}23_P213959), FLJ14712 (A_{—}23_P381505), PCQAP (A_{—}23_P154954), SLC39A7 (A_{—}23_P70571), SPFH1 (A_{—}23_P202029), ITGAL (A_{—}23_P206806), RBX1 (A_{—}23_P211550), CART1 (A_{—}23_P95200), SURB7 (A_{—}23_P2262), MAP7 (A_{—}23_P122736), MGC20533 (A_{—}23_P141965), WDR10 (A_{—}23_P212440), GNL3L (A_{—}23_P22499), (A_{—}23_P426270), CCKBR (A_{—}23_P162007), RIT2 (A_{—}23_P170050), LOC144438 (A_{—}23_P98960), C14orf87 (A_{—}23_P65370), PLD2 (A_{—}23_P4308), CRYM (A_{—}23_P77731), (A_{—}23_P16408), DKFZp7621137 (A_{—}23_P157022), FGF14 (A_{—}23_P88033), (A_{—}23_P256260), GRIK4 (A_{—}23_P116249), CLDN5 (A_{—}23_P6321), KLHL10 (A_{—}23_P27128), COL3A1 (A_{—}23_P142527), RAGAP1L (A_{—}23_P200325), C1orf27 (A_{—}23_P282), KCNK3 (A_{—}23_P91104), ZBED1 (A_{—}23_P217508), (A_{—}23_P90732), Sep-06 (A_{—}23_P22613), GCM2 (A_{—}23_P59285), TH (A_{—}23_P258633), (A_{—}23_P46068), CPN1 (A_{—}23_P98147), VAMP1 (A_{—}23_P105545), NUPL2 (A_{—}23_P123039), NDUFC2 (A_{—}23_P2152), HYAL1 (A_{—}23_P69329), MGC57211 (A_{—}23_P420-431), QKI (A_{—}23_P81759), LOC51204 (A_{—}23_P61738), FLJ90650 (A_{—}23_P58557), EFNA5 (A_{—}23_P167497), VN1R4 (A_{—}23_P78656), TCF2 (A_{—}23_P207557), MRE11A (A_{—}23_P150189), (A_{—}23_P156811), FIBCD1 (A_{—}23_P112187), ZNRF1 (A_{—}23_P163858), GK001 (A_{—}23_P49616), CDADC1 (A_{—}23_P48299), FBXO15 (A_{—}23_P342709), FLJ21019 (A_{—}23_P152755), POLR3B (A_{—}23_P87730), (A_{—}23_P36205), (A_{—}23_P200699), ALLC (A_{—}23_P108734), ADCY8 (A_{—}23_P169989), TMEM41A (A_{—}23_P80831), KIAA1909 (A_{—}23_P81640), PTPN9 (A_{—}23_P124485), CAGLP (A_{—}23_P62588), RNF126 (A_{—}23_P314086), PLEKHG3 (A_{—}23_P76901), C10orf72 (A_{—}23_P304509), CXorf53 (A_{—}23_P217659), FLJ12892 (A_{—}23_P384056), (A_{—}23_P171007), EPN2 (A_{—}23_P89310), KLF11 (A_{—}23_P319512), PLA2G4B (A_{—}23_P403-424), KCNN1 (A_{—}23_P119578), DYRK1A (A_{—}23_P211126), R-spondin (A_{—}23_P22013), CACNG7 (A_{—}23_P4782), SLC22A8 (A_{—}23_P87219), HLA-DMA (A_{—}23_P42304), DEF6 (A_{—}23_P321913), C20orf36 (A_{—}23_P210827), MCM6 (A_{—}23_P90612), OVGP1 (A_{—}23_P103756), (A_{—}23_P217727), MARVELD3 (A_{—}23_P152428), (A_{—}23_P72434), FRMD3 (A_{—}23_P135123), KCND1 (A_{—}23_P217218), LOC134548 (A_{—}23_P400-406), (A_{—}23_P206598), MGC42090 (A_{—}23_P416821), GBF1 (A_{—}23_P161237), ROS1 (A_{—}23_P70278), PTPRH (A_{—}23_P101642), MYLK (A_{—}23_P132428), LOC93349 (A_{—}23_P337753), FMNL1 (A_{—}23_P89365), PIN1L (A_{—}23_P267), ZNF307 (A_{—}23_P133868), MGC4659 (A_{—}23_P61987), ATP11A (A_{—}23_P105908), ILF3 (A_{—}23_P435987), CCBE1 (A_{—}23_P55544), (A_{—}23_P136724), PHF12 (A_{—}23_P305761), ALOX15B (A_{—}23_P60627), WWOX (A_{—}23_P206413), CNOT1 (A_{—}23_P77371), ERBB2 (A_{—}23_P89249), C2GNT3 (A_{—}23_P122077), E2F2 (A_{—}23_P125990), DKFZP566N034 (A_{—}23_P39550), TGM1 (A_{—}23_P65617), SMAP-1 (A_{—}23_P218170), TNK2 (A_{—}23_P324931), FLJ13291 (A_{—}23_P3562), PTK6 (A_{—}23_P56978), PGM2L1 (A_{—}23_P396765), (A_{—}23_P250528), KRT6A (A_{—}23_P87653), LOC93109 (A_{—}23_P428840), SENP3 (A_{—}23_P163997), STK22B (A_{—}23_P40516), PDCD11 (A_{—}23_P161257), ABCA2 (A_{—}23_P43504), (A_{—}23_P155582), MYOM1 (A_{—}23_P96271), GDDR (A_{—}23_P61318), CROT (A_{—}23_P168669), PLEKHA1 (A_{—}23_P115792), (A_{—}23_P26674), MGC20410 (A_{—}23_P370682), CHRM3 (A_{—}23_P401-472), CHRNA10 (A_{—}23_P411188), LGR6 (A_{—}23_P23837), KIAA0514 (A_{—}23_P98115), ABCG4 (A_{—}23_P203231), (A_{—}23_P38978), KIF4A (A_{—}23_P148475), CRYBB1 (A_{—}23_P143621), SELE (A_{—}23_P97112), LOC199675 (A_{—}23_P330561), APOC4 (A_{—}23_P27450), AGTRL1 (A_{—}23_P318860), BACH₂ (A_{—}23_P30633), OR8A1 (A_{—}23_P75677), RNF13 (A_{—}23_P29378), TBX21 (A_{—}23_P141555), VCX2 (A_{—}23_P171188), TP531NP1 (A_{—}23_P168882), BIRC8 (A_{—}23_P90058), SRISNF2L (A_{—}23_P18202), MGC11349 (A_{—}23_P211829), MTM1 (A_{—}23_P62128), NAP1L2 (A_{—}23_P125705), IGSF4C (A_{—}23_P5070), (A_{—}23_P214511), GNB4 (A_{—}23_P18186), FN3KRP (A_{—}23_P77813), CD1A (A_{—}23_P402670), IPO13 (A_{—}23_P384600), (A_{—}23_P44054), SLC37A4 (A_{—}23_P35970), ATR (A_{—}23_P136054), GNAZ (A_{—}23_P416581), ZFPM2 (A_{—}23_P168909), PAPPA (A_{—}23_P351270), BVES (A_{—}23_P502782), MMP15 (A_{—}23_P100177), SFXN2 (A_{—}23_P161253), AQP6 (A_{—}23_P204712), OR6S1 (A_{—}23_P54075), C10orf94 (A_{—}23_P115805), SQSTM1 (A_{—}23_P81401), SEC3L1 (A_{—}23_P113972), (A_{—}23_P104867), OR52J3 (A_{—}23_P53119), POLD2 (A_{—}23_P71140), KIAA0363 (A_{—}23_P93937), INSL5 (A_{—}23_P51479), ZNF76 (A_{—}23_P8133), SOX18 (A_{—}23_P255418), C14orf27 (A_{—}23_P65388), (A_{—}23_P10091), BAPX1 (A_{—}23_P386254), ADRB2 (A_{—}23_P145024), TRIM49 (A_{—}23_P1575), ODZ1 (A_{—}23_P257263), BTD (A_{—}23_P155348), UMODL1 (A_{—}23_P315964), C9orf19 (A_{—}23_P414913), FLJ23834 (A_{—}23_P348253), TNRC5 (A_{—}23_P19352), (A_{—}23_P33429), DKFZP434H2010 (A_{—}23_P23617), LOC220929 (A_{—}23_P161156), PIGC (A_{—}23_P86250), (A_{—}23_P99498), DTNA (A_{—}23_P208158), GPR161 (A_{—}23_P354320), GNRH1 (A_{—}23_P254594), KRTAP3-3 (A_{—}23_P89649), (A_{—}23_P130496), RIP (A_{—}23_P66758),

wherein an increase in the level of expression of at least one gene selected from group (a) and/or (b) and/or (c) and/or (d) and/or wherein a decrease in the level of expression of at least one gene selected from group (e) and/or (f) and/or (g) and/or (h) indicates a reduced level or activity of HDAC2 and is therefore indicative of cancer.

Note that for all genes listed, the corresponding probe identifier as produced by Agilent is also listed in parentheses. This provides a representation of the actual probe utilised in each case. As discussed below (see the experimental section and FIG. 8), all of these genes show significantly altered expression between normal cells and cancer cells in which HDAC2 is truncated (p<0.05).

In one preferred embodiment, the genes assessed are taken from those genes listed in groups (a) and/or (b) and/or (e) and/or (f) above. For these groups of genes the changes in expression are calculated to be highly significant (p<0.01).

Preferably the panel comprises at least one, two, three, four, etc of the genes listed above, up to all genes. All permutations and combinations of the genes listed above are contemplated for gene panels within the scope of the present invention.

For larger panels, use of microarrays may be preferable. Thus, the invention provides, in a second aspect, a microarray for use in the methods of the invention which involve determining levels of HDAC2 indirectly by looking at expression of other genes, comprising probes immobilised on a solid support hybridizing with transcripts or parts thereof of at least one gene selected from those listed (in groups (a) to (h)) above. Preferably, the genes are selected from those genes listed in groups (a) and/or (b) and/or (e) and/or (f) above. For these groups of genes the changes in expression are calculated to be highly significant (p<0.01).

Preferably there are probes immobilised on a solid support hybridizing with transcripts or parts thereof of at least one, two, three, four, etc of the genes listed above, up to all of the genes. All permutations and combinations of the genes listed above are contemplated within the scope of the present invention, for the purposes of providing a microarray.

Microarrays and their means of manufacture are well known and can be manufactured to order by commercial entities such as Agilent and Affymetrix, for example.

The probes are the sequences which are immobilized onto the array, by known methods, and which represent selected sequences from the genes of interest, in this case HDAC2 and/or genes whose expression is affected by a loss or reduced activity or level of HDAC2 in a cell. Probe selection and array design lie at the heart of the reliability, sensitivity, specificity, and versatility of the microarrays of the invention. The methods for selecting suitable probes would be readily apparent for one of skill in the art and may involve optimization using data collected from multiple databases, bioinformatics tools, and experiment-trained computer models.

The key elements of probe selection and design are common to the production of all arrays, regardless of their intended application and as such would be well known to one of skill in the art. Strategies to optimize probe hybridization, for example, may be included in the process of probe selection. Hybridization under particular pH, salt, and temperature conditions can be optimized by taking into account melting temperatures and using empirical rules that correlate with desired hybridization behaviours.

The GeneChip arrays produced by Affymetrix involve a Perfect Match/Mismatch probe strategy. For each probe designed to be perfectly complementary to a target sequence, a partner probe is generated that is identical except for a single base mismatch in its centre. These probe pairs, called the “Perfect Match probe (PM)” and the “Mismatch probe (MM)”, allow the quantitation and subtraction of signals caused by non-specific cross-hybridization. The difference in hybridization signals between the partners, as well as their intensity ratios, serve as indicators of specific target abundance. Such an array design may be applicable to, and incorporated into, the arrays of the present invention.

In order to ensure specificity of the probes in terms of accurately representing the genes whose expression is investigated, the microarray preferably comprises at least 10 probes representing each gene on the array. However, other numbers of probes may be utilised provided that the expression of each gene which is selected to form part of the array can be accurately and specifically measured.

In a preferred embodiment, the array includes probes which represent each and every one of the genes listed. However, this may not be necessary in order to be able to accurately diagnose cancer. Probes representing only one or 2, 3, 4, 5, 6, 7, 8, 9, 10, etc all the way up to all of the genes may be utilised in the array. Accordingly, in one preferred embodiment, the microarray comprises probes representing transcripts of at least the NCOA4 and/or CTSB and/or TBCD and/or PPP2R4 and/or CORO1C gene.

Each probe is preferably at least about 20 nucleotides in length such that a probe of sufficient length to ensure sensitivity and specificity of hybridization is provided. However, any length of probe may be utilised within the scope of the invention, provided that accurate results are achieved in terms of detecting expression of the genes which are representative of HDAC2 levels and thus are useful in the diagnosis of cancer. Possible lengths for the probes include at least 10 nucleotides and up to 250 nucleotides and preferably between about 20 and about 50 nucleotides.

In cases where HDAC2 mRNA is present, reduction or loss of function of the protein encoded by this mRNA may be identified, in one embodiment, by transcribing and cloning its complementary DNA (cDNA). Reverse transcription is used to obtain cDNA from the mRNA, which is inserted into a vector and cloned into host cells in order to express the HDAC2 protein. Functionality of the expressed protein can then be tested using any suitable technique known in the art, including, but not limited to, the techniques described herein.

In an alternative embodiment, the level or activity of HDAC2 may be determined indirectly by assessing the recruitment of HDAC2 to one or more gene promoters. By recruitment is meant the binding of HDAC2 to the promoter region, which may be direct or indirect binding. Thus, if HDAC2 is not recruited to the relevant promoters, this is indicative of a reduced level or activity of HDAC2 and leads to a diagnosis of cancer. In one embodiment, a loss of recruitment of HDAC2 at one or more gene promoters indicates a reduced level or activity of HDAC2 and is therefore indicative of cancer.

In a preferred embodiment, recruitment at any one of the HDAC2, TBCD, PPP2R4 or COROC promoters is monitored. Monitoring of any one, two, three or all four of these promoters together is included within the scope of the invention.

Recruitment of HDAC2 to a particular gene promoter may be measured by any technique known in the art. In one embodiment, chromatin immunoprecipitation is utilised in order to determine whether HDAC2 is present and active and therefore is binding to a particular gene promoter. Chromatin immunoprecipitation is a well known technique in the art which relies upon cross-linking of the binding protein to the DNA, followed by isolation, shearing of the DNA, antibody detection, and isolation by precipitation. The isolated DNA is then released from the binding protein by reversing the cross-linking and is amplified by PCR to determine where the binding protein was bound. (Metivier, R. et al., Estrogen receptor-alpha directs ordered, cyclical, and combinatorial recruitment of cofactors on a natural target promoter, Cell 2003, 115(6) P751-63).

As an alternative or supplementary technique, hyperacetylated chromatin is also associated with a reduction or loss of functionality of HDAC2. This can be quantified by any suitable technique. For example, standard chromatin immunoprecipitation assays may be utilised.

Thus, in one embodiment, analysis of histone acetylation is carried out in order to determine the level or activity of HDAC2. Preferably, an increase in the acetylation levels of one or more histones indicates a reduced level or activity of HDAC2 and is therefore indicative of cancer. In a specific embodiment, the acetylation levels of histone H3 and/or histone H4 are determined. An increase in acetylation of histones H3 and H4 has been shown to be associated with cancer cells in which HDAC2 expression is lost (see FIG. 1g and supplementary FIG. 2d).

By “activity” is meant the enzymatic activity of HDAC2, namely its ability to deacetylate a substrate histone. Any suitable assay may be employed in order to determine the activity of HDAC2 in the sample under test.

To determine HDAC2 enzymatic activity in the sample, it may first be necessary or preferable to isolate HDAC2. Thus, for example, an immunoprecipitation step may be employed, as are well known in the art, using a suitable HDAC2 specific reagent, such as an antibody (see reference 4 of the methods section for example).

A scintillation assay may be employed for example in order to determine HDAC2 activity. This requires a suitably labelled substrate, such as [3H] labelled histones. Alternative labels and substrates may be utilised as appropriate. The sample, or immunoprecipitate may be incubated with the substrate at a suitable temperature, such as 37° C., for a suitable time period, such as 30 minutes, 1 hour, 2 hours etc. If room temperature incubations are carried out, longer reaction times may be required. If HDAC2 is present in the sample or immunoprecipitate, [3H] acetate will be released from the histones and this release may be measured accordingly. The released [3H] acetate may need to be suitably extracted by known means, such as by using ethyl acetate and centrifugation to produce a [3H] acetate containing supernatant, prior to scintillation counting.

For all techniques employed to determine the level or activity of HDAC2, there is preferably included a suitable control sample for comparison. Preferably, both negative and positive controls are included (as discussed in greater detail above).

If desired, it may be possible to identify the HDAC2 mutation at the level of the gene. Various techniques are well known in the art. For example, restriction enzyme digestion and electrophoresis techniques can be employed in combination. Thus, PCR is employed to amplify the HDAC2 gene which may carry the mutation. Genomic DNA can be isolated from a tissue sample from the subject by extraction techniques well known in the art, such as phenol-chloroform extraction, for example. Suitable primers specific for the HDAC2 gene are chosen. The PCR product is then treated with one or more restriction enzymes chosen so that the size of one or more fragments resulting from cleavage of the DNA amplified from mutant HDAC2 differs from the fragment size of DNA amplified from wild-type HDAC2. Fragment sizes may be determined by electrophoresis.

If the mutation does not lead to restriction fragments that can easily be distinguished by size, Southern blotting may be carried out using appropriate labelled DNA probes for example. Alternatively, the gene may be sequenced by techniques known in the art and the sequence compared with that of wild-type HDAC2 in order to identify whether HDAC2 is functionally affected.

### Pharmacogenetic Methods of the Invention

As expounded in the experimental section below, it has been shown that cancer cell lines lacking HDAC2 have altered resistance to the usual antiproliferative and proapoptotic effects of HDAC inhibitors. Specifically, in cancer cells where HDAC2 is deficient, the cells have an increased resistance to the effects of hydroxamic acid based HDAC inhibitors. Thus, the invention provides for the use of HDAC2 mutational status as a pharmacogenetic indicator.

Accordingly, the invention provides a method for predicting the probability of successful treatment of cancer with a hydroxamic acid based HDAC inhibitor comprising, in a sample obtained from a subject, determining the level or activity of HDAC2, wherein a reduced level or activity of HDAC2 is indicative of a low probability of successful treatment.

In particular, a reduced level or activity of HDAC2 is indicative of a low probability of successful treatment using trichostatin A. Additional hydroxamic acids which are contra-indicated according to the invention may include suberoyl hydroxamic acid (SBHA), 6-(3-chlorophenylureido)caproic hydroxamic acid (3-Cl-UCHA), m-carboxycinnamic acid bishydroxylamide (CBHA), suberoylanilide hydroxamic acid (SAHA), azelaic bishydroxamic acid (ABHA), pyroxamide, aromatic sulfonamides bearing a hydroxamic acid group and cyclic-hydroxamic-acid containing peptides.

Similarly, the invention also provides a method of selecting a suitable treatment regimen for cancer comprising, in a sample obtained from a subject, determining the level or activity of HDAC2, wherein a reduced level or activity of HDAC2 indicates that treatment using a hydroxamic acid based HDAC inhibitor is unsuitable.

In particular, a reduced level or activity of HDAC2 indicates that treatment using trichostatin A is unsuitable. Additional hydroxamic acids which are contra-indicated according to the invention may include suberoyl hydroxamic acid (SBHA), 6-(3-chlorophenylureido)caproic hydroxamic acid (3-Cl-UCHA), m-carboxycinnamic acid bishydroxylamide (CBHA), suberoylanilide hydroxamic acid (SAHA), azelaic bishydroxamic acid (ABHA), pyroxamide, aromatic sulfonamides bearing a hydroxamic acid group and cyclic-hydroxamic-acid containing peptides.

In stark contrast to the situation in respect of hydroxamic acid HDAC inhibitors, it has been found that HDAC2 deficient cancers remain susceptible to the action of other HDAC inhibitors, in particular carboxylic acid based HDAC inhibitors.

Accordingly, in one embodiment according to the method described above, a reduced level or activity of HDAC2 leads to treatment using a carboxylic acid based HDAC inhibitor being selected (in preference to use of a hydroxamic acid based HDAC inhibitor).

Thus, in a further aspect of the invention there is provided a method of selecting a suitable treatment regimen for cancer comprising, in a sample obtained from a subject, determining the level or activity of HDAC2, wherein a reduced level or activity of HDAC2 indicates that treatment using a carboxylic acid based HDAC inhibitor should be selected.

In particular, a reduced level or activity of HDAC2 indicates that treatment using valproate and/or butyrate should be selected.

The pharmacogenetic methods of the invention may incorporate any and all of the preferred aspects described in respect of the diagnostic methods described above. In particular, preferably a total loss of, or a significant reduction in, HDAC2 expression and/or activity is investigated. Thus, the absence of HDAC2 expression and/or activity is indicative of cancer and is also indicative that treatment using a carboxylic acid based HDAC inhibitor is positively indicated, whereas treatment using a hydroxamic acid based HDAC inhibitor is contra-indicated. Preferably, the diagnostic methods of the invention are carried out as a prelude to, or as an integral part of, the pharmacogenetic methods of the invention.

Thus, for example, the description of suitable methods for determining levels or activity of HDAC2, suitable test samples, preferred subjects and specific types of cancer which may be monitored all apply mutatis mutandis to these aspects of the invention and are not repeated here simply for reasons of conciseness. Accordingly, according to these aspects of the invention, in a preferred embodiment, the cancer which is to be treated is one which displays microsatellite instability (MSI). The methods of these aspects of the invention may be utilised to select suitable treatment regimens for, or determine the likelihood of successful treatment of, any of colorectal, gastric and/or endometrial cancer. In one preferred embodiment, the methods may be used to select suitable treatment regimens for, or determine the likelihood of successful treatment of, hereditary nonpolyposis colon cancer and/or sporadic colorectal cancer.

### Methods of Treatment and Medical Uses of the Invention

As aforementioned, it has been shown herein for the first time that cancer cell lines lacking HDAC2 have altered resistance to the usual antiproliferative and proapoptotic effects of HDAC inhibitors. Specifically, in cancer cells where HDAC2 is deficient, the cells have an increased resistance to the effects of hydroxamic acid HDAC inhibitors. This resistance is not seen for carboxylic acid based HDAC inhibitors.

In light of these surprising discoveries, the invention provides a method for treating cancer in a subject using carboxylic acid based HDAC inhibitors, the method comprising selecting a subject for treatment according to the diagnostic and/or pharmacogenetic methods of the invention.

In a related aspect, the invention provides for the use of carboxylic acid based HDAC inhibitors in the manufacture of a medicament for treating cancer in a subject, wherein the subject has been selected for treatment according to the diagnostic and/or pharmacogenetic methods of the invention.

Thus, the invention allows, through use of the diagnostic and pharmacogenetic methods of the invention described above, a new patient population which is resistant to treatment with hydroxamic acid based HDAC inhibitors to be selected. This patient population is thus given an alternative treatment, based upon use of carboxylic acid based HDAC inhibitors, which is much more likely to provide successful treatment of their cancer.

Preferably, the carboxylic acid based HDAC inhibitors comprise valproate and/or butyrate. It should be noted that there are a number of suitable HDAC inhibitors in clinical trials and accordingly, the skilled person is aware of suitable formulations etc which may be utilised.

In similar fashion, the invention also provides a method for treating cancer in a subject using hydroxamic acid based HDAC inhibitors, the method comprising selecting a subject for treatment according to the diagnostic and/or pharmacogenetic methods of the invention. Thus, if HDAC2 activity is present, treatment using a hydroxamic acid based HDAC inhibitor is possible.

In a related aspect, the invention provides for the use of hydroxamic acid based HDAC inhibitors in the manufacture of a medicament for treating cancer in a subject, wherein the subject has been selected for treatment according to the diagnostic and/or pharmacogenetic methods of the invention.

Thus, the invention allows, through use of the diagnostic and pharmacogenetic methods of the invention described above, a new patient population which remains susceptible to treatment with hydroxamic acid based HDAC inhibitors to be selected. This patient population may thus be recommended a treatment regiment based upon use of hydroxamic acid based HDAC inhibitors, which has the potential to provide successful treatment of their cancer.

In one embodiment, the presence of HDAC2 indicates that treatment using trichostatin A remains possible. Additional hydroxamic acids include suberoyl hydroxamic acid (SBHA), 6-(3-chlorophenylureido)caproic hydroxamic acid (3-Cl-UCHA), m-carboxycinnamic acid bishydroxylamide (CBHA), suberoylanilide hydroxamic acid (SAHA), azelaic bishydroxamic acid (ABHA), pyroxamide, aromatic sulfonamides bearing a hydroxamic acid group and cyclic-hydroxamic-acid containing peptides.

It should be noted that, for the purposes of the present invention, the designation of a particular HDAC inhibitor is considered to encompass all pharmaceutically acceptable forms of the active compound which are useful as HDAC inhibitors. Thus, stereoisomers, enantiomers, salts, esters etc are all encompassed within the scope of the invention as appropriate.

Thus, in a pharmaceutical composition incorporating a suitable HDAC inhibitor, preferred compositions include pharmaceutically acceptable carriers including, for example, non-toxic salts, sterile water or the like. A suitable buffer may also be present allowing the compositions to be lyophilized and stored in sterile conditions prior to reconstitution by the addition of sterile water for subsequent administration. The carrier may also contain other pharmaceutically acceptable excipients for modifying other conditions such as pH, osmolarity, viscosity, sterility, lipophilicity, somobility or the like. Pharmaceutical compositions which permit sustained or delayed release following administration may also be used.

Suitable pharmaceutical compositions for use in the treatment methods or medical uses of the invention may be used together with other standard chemotherapeutic treatments which target tumour cells directly. Non limiting examples include paclitaxel, cyclaphosphomide and 5-tumor-uracil (5-FU) and pharmaceutically acceptable derivatives thereof including salts, etc.

In one embodiment, the pharmaceutical composition, preferably comprising a carboxylic acid based HDAC inhibitor, for use in the treatment methods or medical uses of the invention is in a form suitable for metronomic dosing.

The therapeutic agent may, for example, be encapsulated and/or combined with suitable carriers in solid dosage forms for oral administration which would be well known to those of skill in the art or alternatively with suitable carriers for administration in an aerosol spray. Examples of oral dosage forms include tablets, capsules and liquids.

Alternatively, the therapeutic agent may be administered parenterally. Specific examples include intradermal injection, subcutaneous injection (which may advantageously give slower absorption of the therapeutic agent), intramuscular injection (which can provide more rapid absorption), intravenous delivery (meaning the drug does not need to be absorbed into the blood stream from elsewhere), sublingual delivery (for example by dissolving of a tablet under the tongue or by a sublingual spray), rectal delivery, vaginal delivery, topical delivery, transdermal delivery and inhalation.

Furthermore, as would be appreciated by the skilled practitioner, the specific dosage regime may be calculated according to the body surface area of the patient or the volume of body space to be occupied, dependent on the particular route of administration to be used. The amount of the composition actually administered will, however, be determined by a medical practitioner based on the circumstances pertaining to the disorder to be treated, such as the severity of the symptoms, the age, weight and response of the individual.

The methods of treatment and medical uses according to the invention described supra may incorporate any and all of the preferred aspects described in respect of the diagnostic and pharmacogenetic methods described above. Preferably, the diagnostic methods and/or the pharmacogenetic methods of the invention are carried out as a prelude to, or as an integral part of, the methods of treating cancer according to the invention.

Thus, for example, the description of suitable methods for determining levels or activity of HDAC2, suitable test samples, preferred subjects and specific types of cancer which may be monitored all apply mutatis mutandis to these aspects of the invention and are not repeated here simply for reasons of conciseness.

According to the treatment method aspects of the invention, in a preferred embodiment, the cancer which is treated in the patient subpopulation identified according to the diagnostic and/or pharmacogenetic methods of the invention is one which displays microsatellite instability (MSI). These methods of the invention may be utilised to treat any of colorectal, gastric and/or endometrial cancer. In one preferred embodiment, the methods may be used to treat hereditary nonpolyposis colon cancer and/or sporadic colorectal cancer.

### Gene Therapy Methods

With the realisation of HDAC2's role as a tumour suppressor gene, whose functional abrogation is linked to specific cancers, there is the possibility of restoring HDAC2 functionality in order to treat cancer. As shown in the experimental section below, transfection of HDAC2 deficient cells, including HDAC2 deficient cancer cells, with a suitable HDAC2 containing vector induces tumour suppressor-like features.

Accordingly, in a still further aspect, the invention provides a method for treating cancer in a subject, said subject displaying a reduced level or activity of HDAC2, comprising reconstitution of HDAC2 activity in the subject.

In a preferred embodiment, the cancer has been diagnosed or assessed according to the diagnostic and pharmacogenetic methods of the invention. This method may, in a further embodiment, be used in conjunction with the other treatment methods and medical uses described herein.

Thus, also provided is the use of a vector carrying the HDAC2 gene in the manufacture of a medicament for treating cancer in a subject, wherein the subject has been selected for treatment according to the diagnostic and/or pharmacogenetic methods of the invention.

Preferably, the reconstitution of HDAC2 activity comprises introducing wild type copies of the HDAC2 gene into the subject.

Any suitable vector for delivery of functional copies of the HDAC2 gene may be utilised according to the method of the invention. One principal requirement is that tissue specificity of delivery and expression is achieved. The two major sources of vectors which may be utilised comprise viral vectors and non-viral vectors.

Within the group of viral vectors, preferred types include adenoviruses, retroviruses, in particular Moloney murine leukaemia virus (Mo-MLV), adeno-related viruses and herpes simplex virus type I. Typically, the gene of interest, in this case encoding HDAC2, will be included in the viral genome, preferably in the “non-essential” region of the viral genome. In addition, it is important to remove virally encoded protooncogenes from the viral vector genome. The virus may be made replication incompetent to prevent unwanted replication once the virus has been targeted.

In terms of targeting the viral vector to the desired site, a number of possibilities exist. For example, the env gene (which encodes the viral vector's envelope) may be engineered or replaced with the env gene from a different virus to alter the range of cells the viral vector will “infect”. Furthermore, alteration of the viral tropism may be achieved by using suitable antibodies raised against antigenic determinants on the cell surface of the desired target cells. The antibodies, which include all derivatives thereof, such as scFV, nanobodies, VH domains, Fab fragements etc., may be genetically incorporated into the viral vectors to provide targeted gene delivery of the HDAC2 gene. Most preferred is use of scFV (Hedley et al., Gene Therapy (2006) 13, 88-94). The viral vectors may have many genes removed, such as packaging genes, in order to reduce immunogenicity and/or infectivity. These functions may thus be supplied by a helper virus.

Due to their high efficiency of integration, low pathogenicity and high efficacy, adenoviruses are a preferred vector according to the methods of the invention.

Alternatives to viral vectors include direct gene delivery, use of other delivery agents and use of molecular conjugates. Tissue specific promoters may be employed as appropriate. Direct gene delivery may be achieved for example by microinjection of a suitable vector, such as a plasmid carrying the HDAC2 gene, directly into the tissue of interest. Alternatives include use of ballistic transformation, for example using vector coated onto suitable particles (e.g. gold particles). Additional delivery agents include liposomes and derivatives thereof. As discussed above, targeting proteins such as antibodies and derivatives thereof may be utilised in order to ensure delivery to the cells of interest. Molecular conjugates may include suitable proteins conjugated to the DNA of interest using a suitable DNA binding agent.

The methods of treatment and medical uses according to The gene therapy aspects of the invention may incorporate any and all of the preferred aspects described in respect of the diagnostic and pharmacogenetic methods and also methods of treating cancer as described above. Preferably, the diagnostic methods and/or the pharmacogenetic methods of the invention are carried out as a prelude to, or as an integral part of the methods of treating cancer according to the gene therapy aspects of the invention.

Thus, for example, the description of suitable methods for determining levels or activity of HDAC2, suitable test samples, preferred subjects and specific types of cancer which may be treated all apply mutatis mutandis to these aspects of the invention and are not repeated here simply for reasons of conciseness.

According to the gene therapy treatment method aspects of the invention, in a preferred embodiment, the cancer which is treated, preferably in the patient subpopulation identified according to the diagnostic and/or pharmacogenetic methods of the invention, is one which displays microsatellite instability (MSI). These methods of the invention may be utilised to treat any of colorectal, gastric and/or endometrial cancer. In one preferred embodiment, the methods may be used to treat hereditary nonpolyposis colon cancer and/or sporadic colorectal cancer.

### Kits

The invention also provides kits which may be used in order to carry out the methods of the invention. The kits may incorporate any of the preferred features mentioned in connection with the methods of the invention above.

### Kits for Use in Diagnostic Methods

Kits for use in the diagnostic methods of the invention may incorporate suitable means for obtaining a sample. They may also incorporate suitable means for safely handling this sample.

Kits for determining the level or activity of HDAC2 will typically include one or more reagents specific for HDAC2. Preferably, the reagent includes an antibody or an HDAC2 binding derivative thereof. Suitable derivatives are widely known in the art and include Fab fragment, scFv fragments, VH domains, nanobodies, heavy chain antibodies etc. Polyclonal and monoclonal antibodies may be included in the kits of the invention. Other reagents may include any molecule which binds specifically to HDAC2. For example the reagent could be based around a labelled version of an HDAC inhibitor such as trichostatin A for example.

Kits may include the necessary components for immunoprecipitation of HDAC2 followed by the components necessary to monitor HDAC2 activity. Such components are well known in the art. Kits for use in western blotting, immunofluorescence, agglutination assays, radioimmunoassay, flow cytometry and equilibrium analysis are also contemplated within the scope of the invention.

In one embodiment, an ELISA kit contains a suitable chromogenic or chemiluminescent substrate, together with an HDAC2 specific reagent, preferably an antibody (monoclonal or polyclonal or target binding derivative thereof) in order to detect if HDAC2 is present in the sample.

Suitable control enzymes or other markers may also be analysed in the methods of the invention, to confirm the method is working in a satisfactory manner. Thus, the kits may also incorporate suitable reagents specific for the identification of these control enzymes or other markers.

Where the methods of the invention incorporate tests on suitable control samples (both positive and negative controls) the kits will contain sufficient reagents to test all of the controls and the test samples. The reagents may be separately packaged and aliquoted to allow for individual tests to be carried out on both test and control samples using the same kit.

If determining HDAC2 expression, or if indirect gene expression is being utilised in order to determine HDAC2 activity, at the mRNA level, kits may incorporate gene specific primers and/or probes. The kits may further comprise RNA isolation reagents, polymerase enzymes for amplification, buffers etc. The kits may also incorporate suitable RNase inhibitors to prevent degradation of any isolated RNA molecules.

Kits for use in RT-PCR applications may additionally include a reverse transcriptase enzyme together with suitable buffers. Appropriate mixtures of nucleotides, as would be well known to one of skill in the art, may also be included in the kits to facilitate amplification of the template molecules (both RNA to cDNA and amplification of the cDNA).

In a northern blotting embodiment, the kits may include suitable probes which cross react with the appropriate gene. Such probes may be labelled as appropriate, for example with a radiolabel or mass label or fluorescent label.

As mentioned above, the methods of the invention may incorporate nucleic acid amplification techniques. As aforementioned preferred amplification techniques include PCR, nested PCR, Rolling circle replication, NASBA, 3SR, ligase chain reaction (LCR), selective amplification of target polynucleotide sequences, consensus sequence primed polymerase chain reaction, arbitrarily primed polymerase chain reaction, nick displacement amplification and TMA techniques. In the case of nucleic acid amplification techniques, well known in the art, sequence specific primers are required to allow specific amplification of the product with minimal production of false positive results. To this end, the kits of the invention may preferably include sequence specific primers.

The kit may also include reagents necessary for a nucleic acid amplification step. Reagents may include, by way of example and not limitation, amplification enzymes, probes, positive control amplification templates, reaction buffers etc. For example, in the PCR method of amplification, possible reagents include a suitable polymerase such as Taq polymerase and appropriate PCR buffers, and in the TMA method the appropriate reagents include RNA polymerase and reverse transcriptase enzymes. All of these reagents are commercially available and well known in the art.

The kit may further include components required for real time detection of amplification products, such as fluorescent probes for example. As aforementioned the relevant real-time technologies, and the reagents required for such methods, are well known in the art and are commercially available. Thus, for example using the TAQMAN® technique, the probes may need to be of sequence such that they can bind between PCR primer sites on the nucleic acid molecule of interest that is subsequently detected in real-time. Similarly, MOLECULAR BEACONS probes may be designed that bind to a relevant portion of the relevant nucleic acid sequence. If using the SCORPION probe technique for real time detection the probe will need to be designed such that it hybridizes to its target only when the target site has been incorporated into the same molecule by extension of the tailed primer. In the AMPLIFLUOUR method, the primers will be suitably labelled with an appropriate probe. Suitable probes are accordingly included in a further aspect of the kits of the invention.

Kits for use in methods where recruitment to a promoter, or levels of histone acetylation are measured, may include suitable components necessary for carrying out a chromatin immunoprecipitation.

### Pharmacogenetic Kits

These kits may include all the components listed above, since determining the level or activity of HDAC2 is also critical to this aspect of the invention.

Once the level or activity of HDAC2 has been determined, it is then possible to conclude which type of treatment is suitable or not. Accordingly, a suitable information sheet may be incorporated in the kit which allows the user of the kit to interpret the results to thus decide on an appropriate course of treatment. The sheet may take the form of written instructions, or a flow chart or decision tree for example.

### Gene Therapy Kits

Kits for use in the gene therapy aspects of the invention may include a suitable HDAC2 containing construct which allows expression levels of HDAC2 to be restored to normal levels.

The construct is preferably an expression vector which drives expression of HDAC2 in the targeted tissue, which may be a tumour. The types of cancer which are relevant are discussed in detail supra.

The expression vector preferably contains a wild type copy of the HDAC2 gene. However, altered version may be utilised provided they retain substantially wild type, or improved, HDAC activity.

Any suitable vector for delivery of functional copies of the HDAC2 gene may be included in the kits of the invention.

One principal requirement is that tissue specificity of delivery and expression is achieved. The two major sources of vectors which may be utilised comprise viral vectors and non-viral vectors.

Within the group of viral vectors, preferred types include adenoviruses, retroviruses, in particular Moloney murine leukaemia virus (Mo-MLV), adeno-related viruses and herpes simplex virus type I. Typically, the gene encoding HDAC2, will be included in the viral genome, preferably in the “non-essential” region of the viral genome. The virus may be made replication incompetent to prevent unwanted replication once the virus has been targeted.

In terms of targeting the viral vector to the desired site, a number of possibilities exist. For example, the env gene (which encodes the viral vector's envelope) may be engineered or replaced with the env gene from a different virus to alter the range of cells the viral vector will “infect”. Furthermore, alteration of the viral tropism may be achieved by using suitable antibodies raised against antigenic determinants on the cell surface of the desired target cells. The antibodies, which include all derivatives thereof, such as scFV, nanobodies, heavy chain antibodies, VH domains, Fab fragements etc., may be genetically incorporated into the viral vectors to provide targeted gene delivery of the HDAC2 gene. Most preferred is use of scFV (Hedley et al., Gene Therapy (2006) 13, 88-94). The viral vectors may have many genes removed, such as packaging genes, in order to reduce immunogenicity and/or infectivity. These functions may thus be supplied by a helper virus, and kits further including a suitable helper virus are contemplated within the scope of the invention.

Adenoviruses are a preferred vector for inclusion in the kits of the invention.

Alternatives to viral vectors include direct gene delivery, use of other delivery agents and use of molecular conjugates. Tissue specific promoters may be employed as appropriate. Direct gene delivery may be achieved for example by microinjection of a suitable vector, such as a plasmid carrying the HDAC2 gene, directly into the tissue of interest. Kits for direct delivery are included in the scope of the invention. Alternatives include use of ballistic transformation, for example using vector coated onto suitable particles (e.g. gold particles). Additional delivery agents include liposomes and derivatives thereof. As discussed above, targeting proteins such as antibodies and derivatives thereof may be utilised in order to ensure delivery to the cells of interest. Molecular conjugates may include suitable proteins conjugated to the DNA of interest using a suitable DNA binding agent. Kits for use in all of these techniques are envisaged.

Thus, the kit may also include reagents to facilitate transfection, as appropriate.

Since, in a preferred embodiment, the cancer has been diagnosed or assessed according to the diagnostic and pharmacogenetic methods of the invention, the gene therapy kits may also incorporate components from these kits (as described supra).

The invention will now be described with respect to the following non-limiting examples in which:

### BRIEF DESCRIPTION OF THE DRAWINGS

FIG. 1. Biochemical and biological effects of HDAC2 mutations in human cancer.

(a) Schematic representation of the HDAC2 gene, with the location of the (A)₉ repeat, and the amino acid sequence of wild type and mutant HDAC2 proteins. Black and grey arrows represent the transcription and translation start site respectively.

(b) Electropherograms of HDAC2 wild-type (normal colon and SW48) and mutant (RKO and C0115) cells.

(c) HDAC2 protein expression analyzed by western-blot (left) and immunofluorescence (right) is lost in the mutant RKO and C0115 cells, but not in the other colon cancer cell lines with a wild-type sequence. HDAC1 protein and HDAC2 mRNA levels cells are not significantly different.

(d) HDAC2 enzymatic activity analyzed in the HDAC2-immunoprecipitated extracts is deeply depleted in RKO cells.

(e) Quantification of histone H3 and H4 acetylation levels using western-blot (left) and high performance capillary electrophoresis (right) after treatment with HDAC inhibitors. The hydroxamic acid TSA does not induce histone hyperacetylation in the HDAC2-deficient RKO and C0115 cells, whilst in HDAC2-proficient cells (HCT-116, SW48 and LoVo) hyperacetylation is achieved. The carboxylic acids valproate (Val) and butyrate (But) induce hyperacetylation in all cells.

(f) TSA induces cell growth inhibition, apoptosis and G2/M cell cycle arrest in HDAC2-proficient cells, but HDAC2-mutant cells (RKO) show a marked resistance to the these three typical effects of the administration of a HDAC inhibitor.

(g) Butyrate induced tumor shrinkage in all cancer cells xenografted in nude mice independently of their HDAC2 status, whereas TSA was only able to accomplish these effects in the HDAC2-proficient cell line (HCT-116), whilst RKO and CO115 remained resistant (upper right).

Left, unsupervised clustering expression analysis discriminates between HDAC2-mutant (RKO and CO115) and HDAC2-wild-type (HCT-15, LoVo, HCT-116 and SW48) cell lines. Lower right, chromatin immunoprecipitation demonstrates that the loss of HDAC2 occupancy at a particular promoter (NCOA4) in HDAC2-mutant cells (CO115 and RKO) is associated with overexpression of the corresponding gene.

(h) HDAC2 mutations in human cancer.

Immunohistochemistry of HDAC2 in sporadic MSI+colon tumours: Upper, strong HDAC2 expression in two tumours with wild-type sequences; Lower, loss of HDAC2 staining in two tumours harbouring the HDAC2 mutation.

(i) FISH analysis of HDAC2. Metaphases spreads from HCT-116, RKO and CO115 cell lines show two copies of chromosome 6 with green signals (labelled as B in the figure) that correspond to the clone covering the HDAC2 gene and red signals (labelled as A in the figure) that correspond to the control BAC clone mapping to the 6p21 region.

FIG. 2. HDAC2 haploinsufficiency in two endometrial cancer cell lines with HDAC2 heterozygous mutations.

a, Metaphases spreads from AN3CA and SKUT1 cell lines show two copies of chromosome 6 with green signals (labelled as B in the figure) that correspond to the clone covering the HDAC2 gene and red signals (labelled as A in the figure) that correspond to the control BAC clone mapping to the 6p21 region.

b, Western blot showing the levels of HDAC2 expression in SKUT1, AN3CA, RKO and HCT116.

c, Bisulfite genomic sequencing of the HDAC2 promoter gene demonstrates an unmethylated CpG island. A schematic representation of the CpG sites included in the PCR fragment is shown. CpG sites are represented as squares: black (methylated) and white (unmethylated).

d, Quantification of histone H3 and H4 acetylation levels using western blot (left) and high performance capillary electrophoresis (right) after treatment of SKUT1 cells with TSA and sodium valproate.

e, Effects of TSA on cell viability (MTT assay) of SKUT1 cells.

FIG. 3. Response of colon cancer cell lines to TSA.

a, HDAC2 activity in immunoprecipitated extracts of HCT116 and C0115 colon cancer cell lines.

b, Effects of the HDAC inhibitor TSA on cell viability (MTT assay) of the same cell lines. Cells were treated with 250 nM TSA for 48 h

c, Dose response curves of the effects of the HDAC inhibitor TSA on cell viability (MTT assay) of colon cancer cell lines. Cells were treated for 48 h with different concentrations of TSA (0-1000 nM). Experiments were performed in triplicate. The HDAC2-deficients cells lines RKO and C0115 show an enhanced resistance to cell growth inhibition by TSA.

FIG. 4. Tumor xenografts from colon cancer cell lines with HDAC2-mutations (RKO and C0115) are resistant to TSA actions.

a, PBS, BUT and TSA treated female athymic nude mice 17 days after injection of RKO (left flank) and HCT116 (right flank) cells.

b, Tumors were excised and weighed. Tumor detail in cm and weight in mg for PBS-, BUT- and TSA-HCT116, RKO and C0115 cells.

c, Effect of the HDAC inhibitors TSA and sodium butyrate (But) on the in vivo growth of HCT116, RKO and C0115. Tumor size was monitored over time and size in mm3. Dark square: PBS treatment, Dark circle: TSA treatment, Dark triangle: BUT treatment.

FIG. 5. Effects of the HDAC inhibitor suberoylanilide hydroxamic acid (SAHA) on cell viability (MTT assay) and apoptosis (propidium iodide) of colon cancer cell lines. Cells were treated for 24 h with 5 mM SAHA. Experiments were performed in triplicate. The HDAC2-deficient cell line RKO shows an enhanced resistance to cell growth inhibition and apoptosis by SAHA.

FIG. 6: Reconstitution and interference of HDAC2 functions.

a, HDAC2 activity of immunoprecipitated extracts of RKO transfected with empty vector or HDAC2.

HDAC2 and HDAC1 expression levels are shown in the western blot below.

b, Quantification of H4 acetylation by HPCE. RKO-HDAC2-transfected cells show resistance to histone acetylation mediated by TSA.

c, Reduction in colony formation in RKO cells transfected with HDAC2.

d, Reduction in tumor growth in xenografted nude mice in RKO cells transfected with HDAC2.

e, Left, Western blot of HDAC2-knocked down HCT116 cells by siRNA. Right, Quantification of histone H4 acetylation by HPCE. HDAC2-knocked down HCT116 cells are resistant to histone acetylation mediated by TSA.

f, Effect of HDAC silencing by siRNA on the in vivo growth of HCT116 cells. Large tumor on the right flank corresponding to HDAC2-Knocked down HCT116 cells and small tumor on the left flank corresponding to HCT116 cells. Tumor size was monitored over time and size in mm3

FIG. 7: HDAC2 mutant cells have a characteristic expression signature.

a, Chromatin immunoprecipitation (ChIP) analysis of the occupancy by HDAC2 of several promoters obtained from the microarray expression studies. HDAC2 is present in the HCT-116 HDAC2-wild-type cells, but absent in C0115 and RKO. The bound fraction of the ‘no antibody’ (NAB) control is shown as negative control.

b, RT-PCR expression analysis of the HDAC2-target genes demonstrates gene overexpression in HDAC2 mutant cells.

c, Dendrogram representing the cluster analysis of 12 colon tumours with microsatellite instability and normal controls generated by the SOTA software (http://bioinfo.cnio.es/cgibin/tools/cluster-ing/sotarray). The HDAC2-mutant tumors show a distinct pattern of gene expression compared to HDAC2-wild type tumors

FIG. 8. The data produced from the gene expression analysis is reproduced in full in the table shown in FIG. 3. Note that all probes were supplied by Agilent and the probe numbers are designated accordingly.

### EXPERIMENTAL SECTION

### 1. Introduction

Widespread changes in DNA methylation^{1,2} and post-translational modifications of histones occur in cancer cells^{3,4}, and both marks have a crucial role in chromatin packaging and gene expression^{1,2,5,6}. We are largely ignorant of the mechanisms underlying the disruption of the epigenetic landscape in transformed cells. One interesting possibility is that the enzymes that epigenetically modify DNA and histones, and the transcription factors that “read” these marks, may themselves be targets of genetic disruption, such as it occurs with the p300 histone acetyltransferase^{7,8}.

To explore the presence of inactivating mutations in the described “epigenetic modifier genes” it is useful to consider tumors displaying microsatellite instability (MSI), both in the context of hereditary nonpolyposis colon cancer (HNPCC) associated with germline mutations in the mismatch repair genes⁹, and in sporadic cancers associated with hMLH1 inactivation by promoter CpG-island methylation^{9,10}. Tumors with MSI progress along a genetic pathway that exhibits a high rate of insertion/deletion mutations in mononucleotide repeats, which often result in the generation of premature stop codons. Illustrative target genes include the growth-control gene TGFBRII¹¹ and the proapoptotic gene BAX¹²

### 2. Results

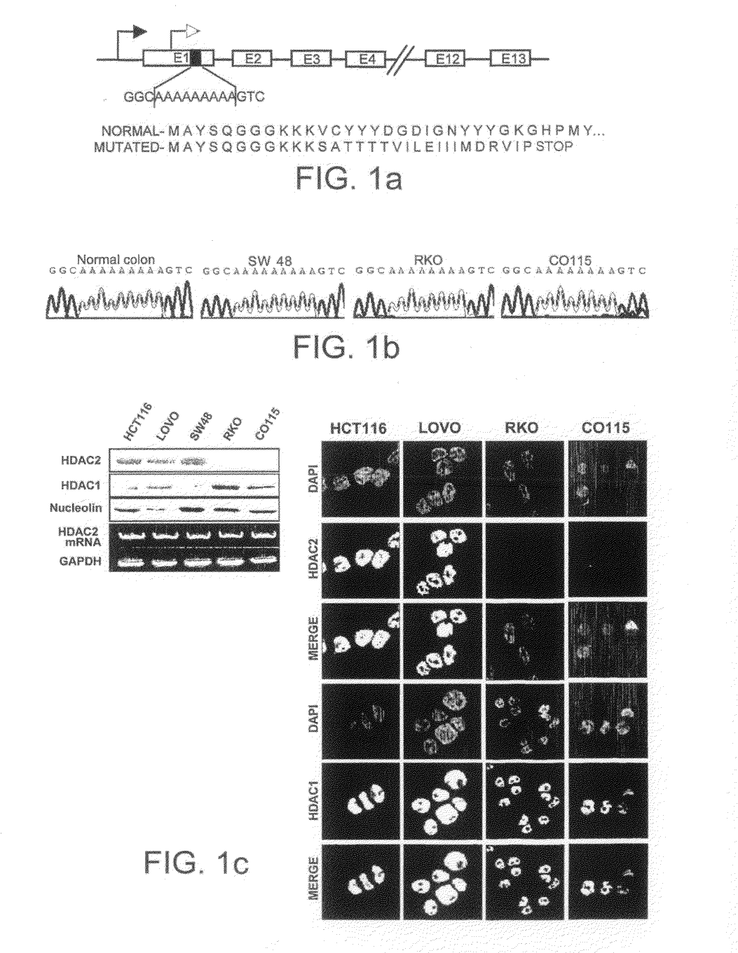

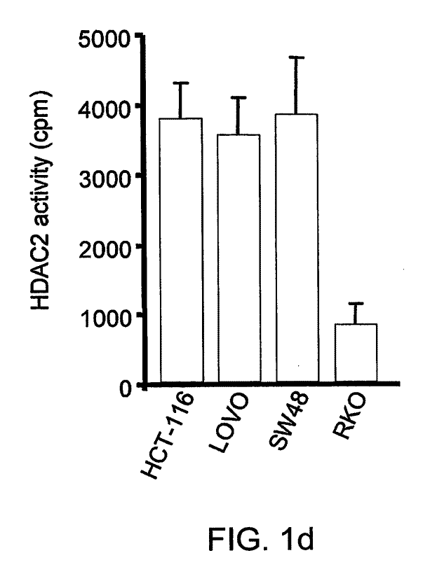

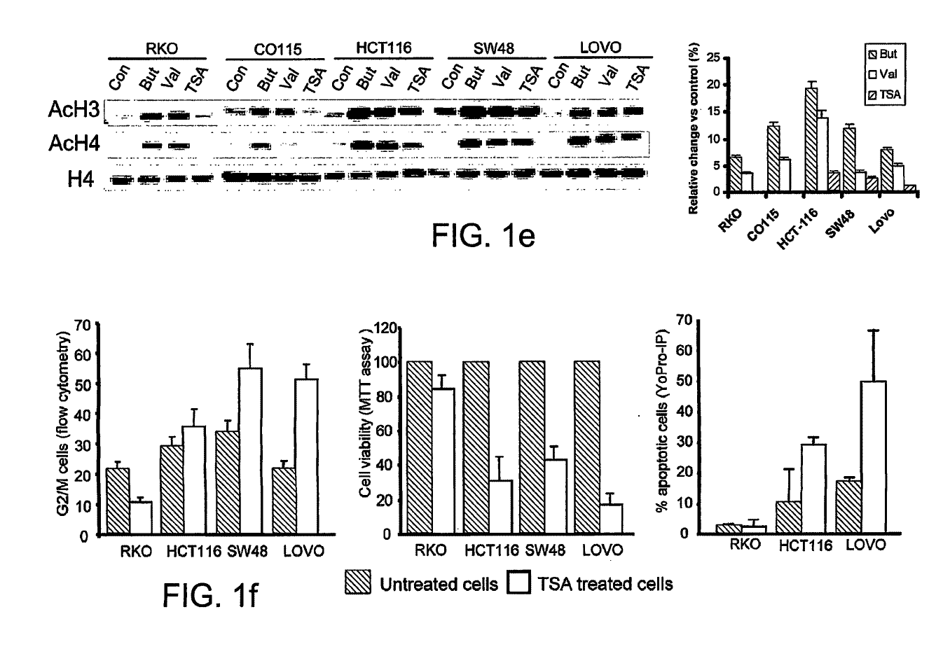

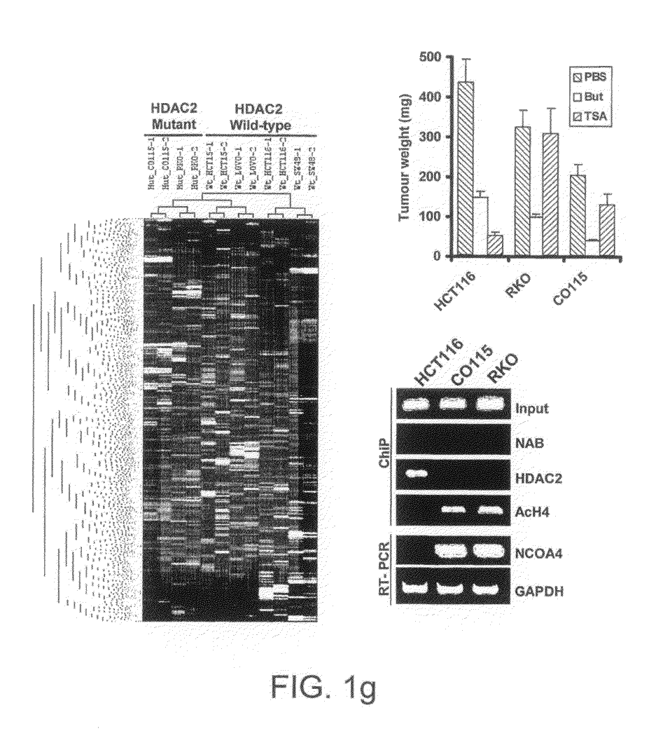

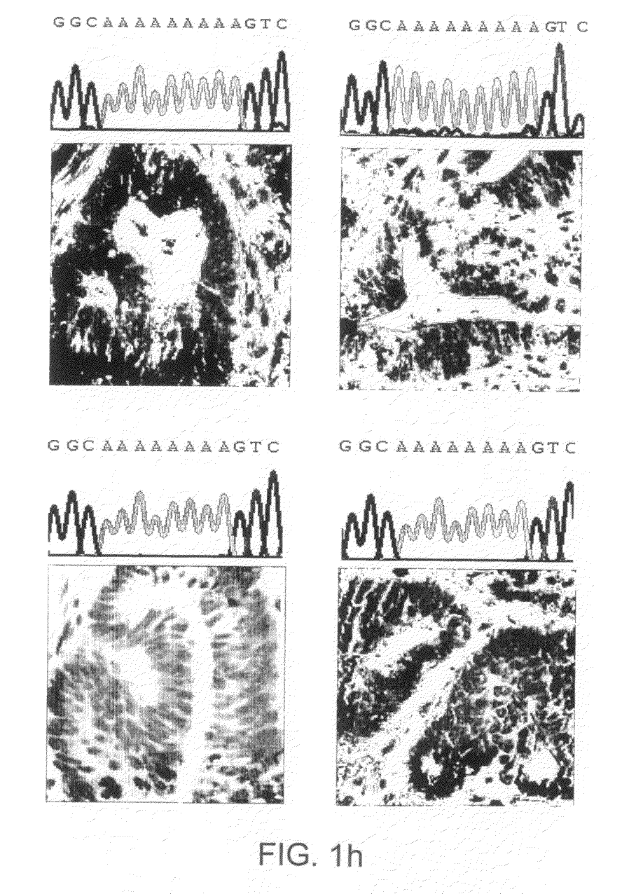

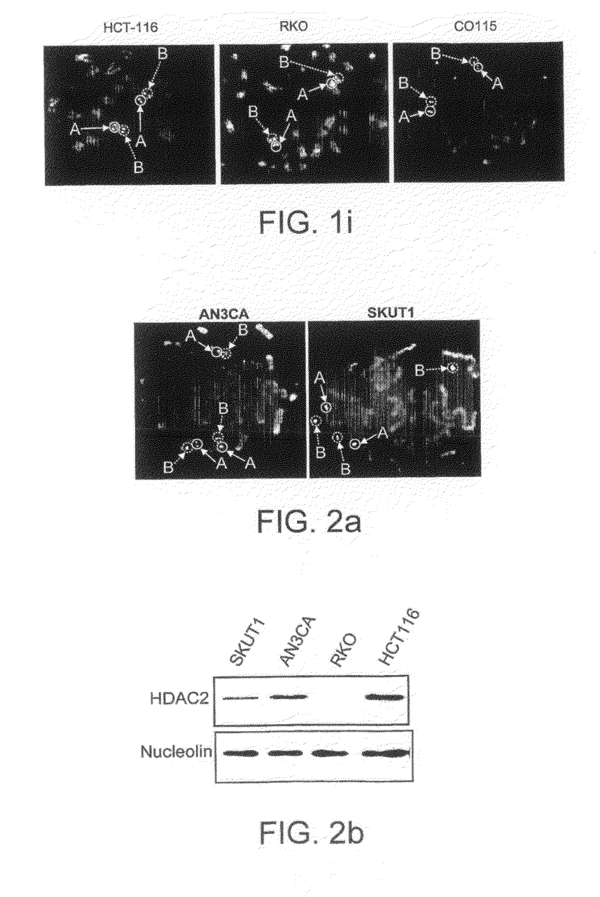

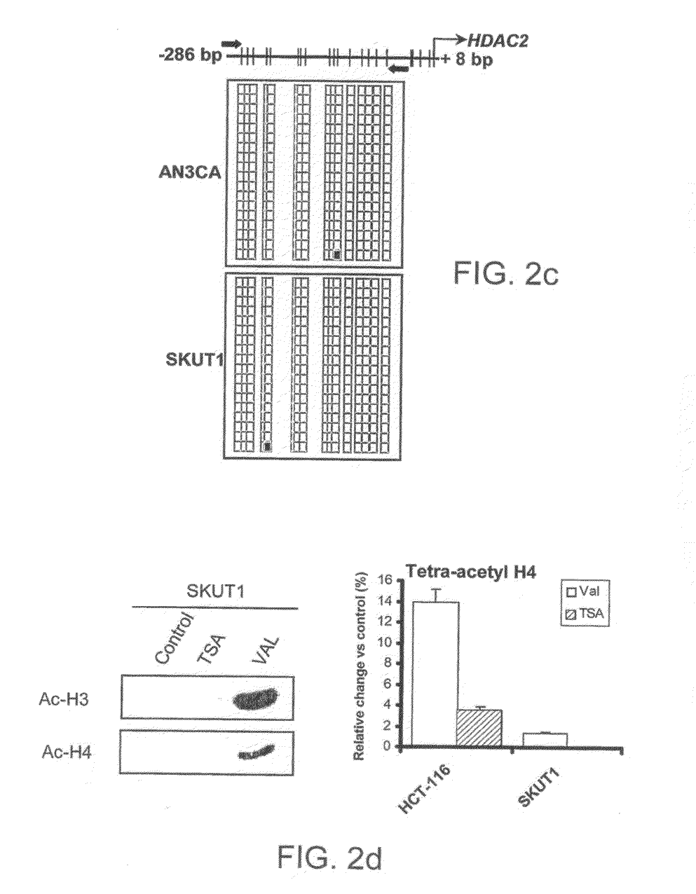

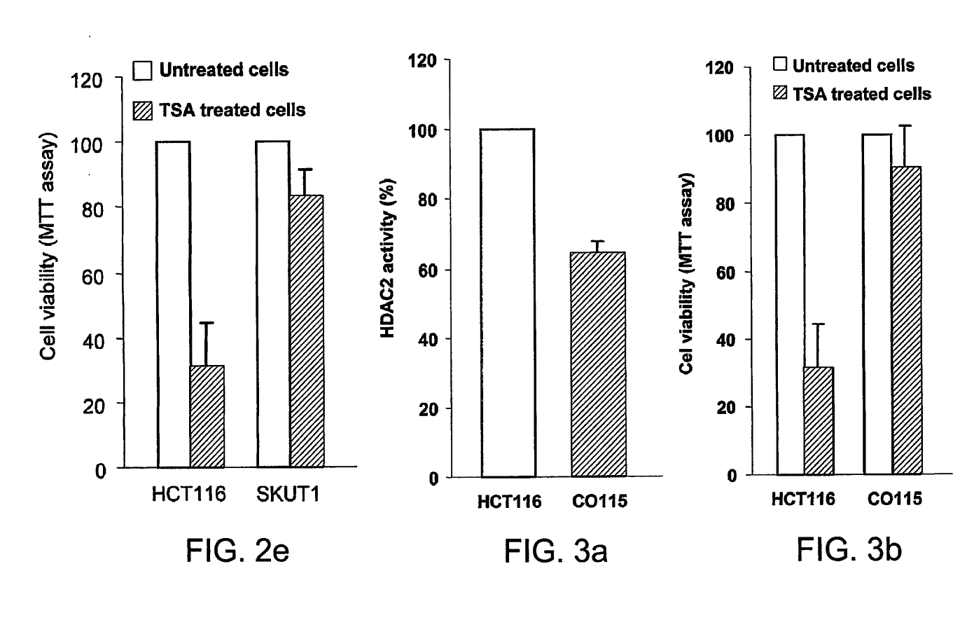

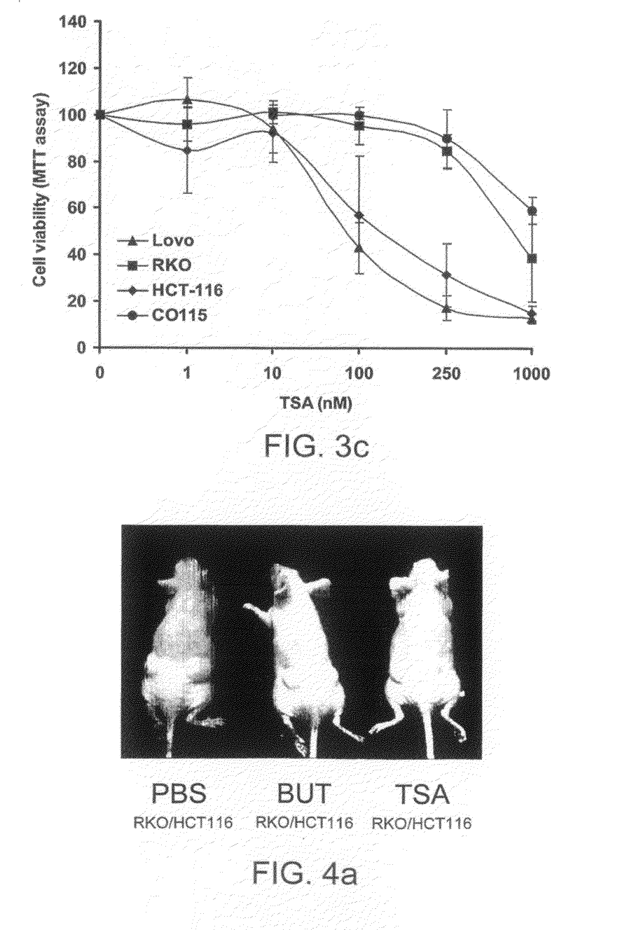

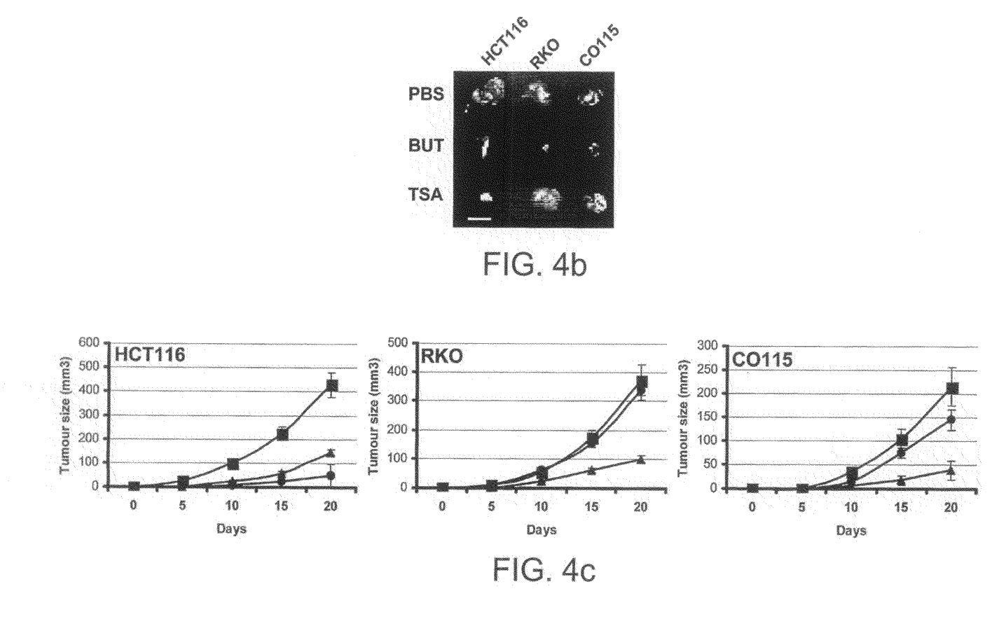

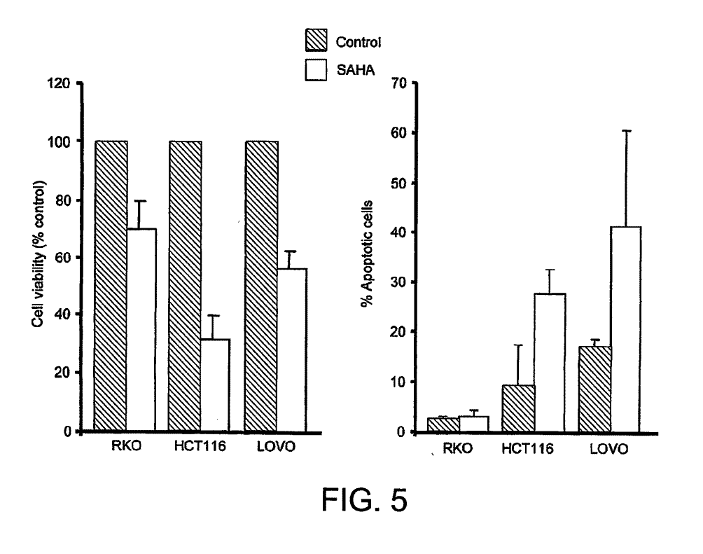

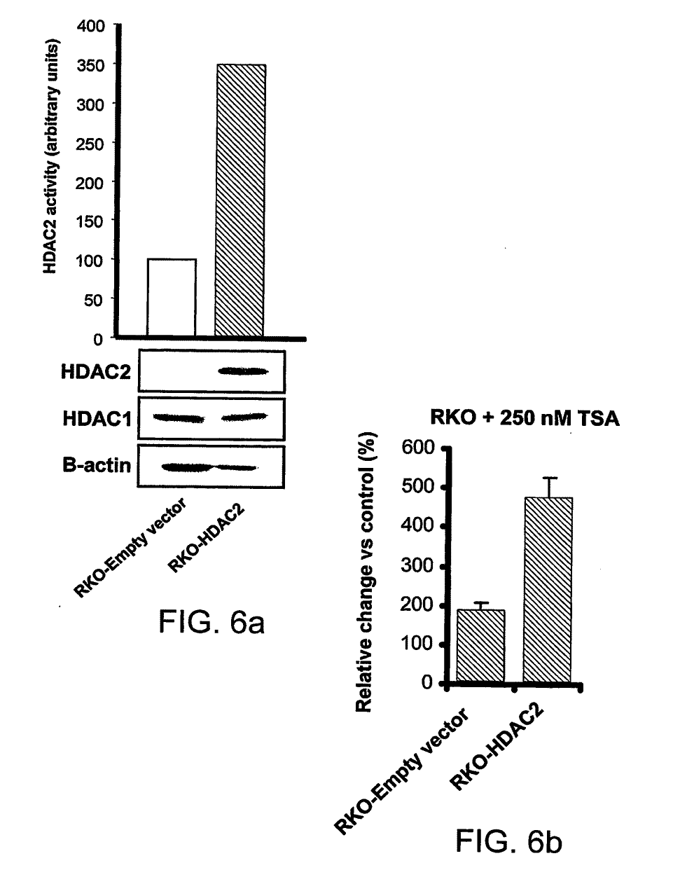

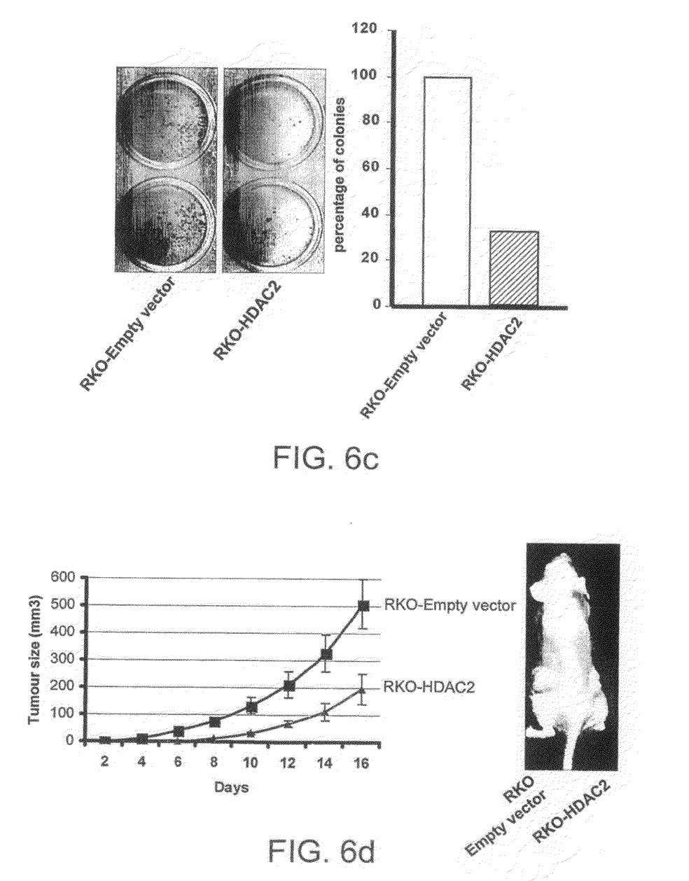

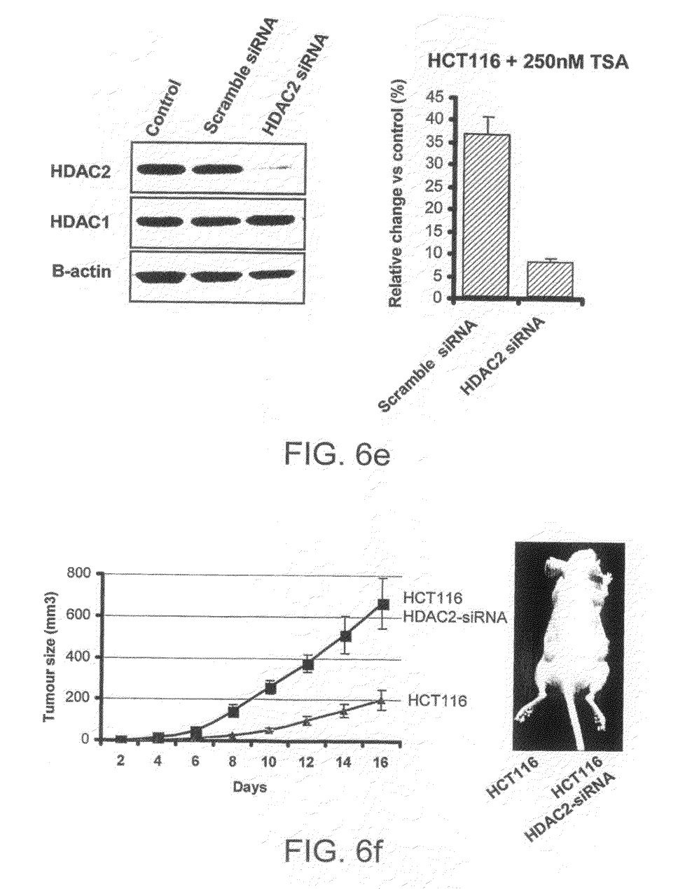

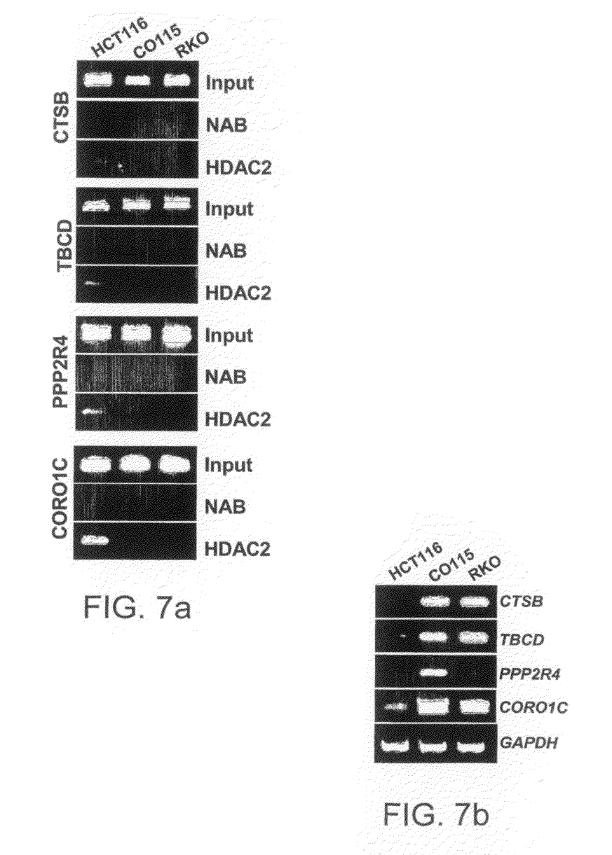

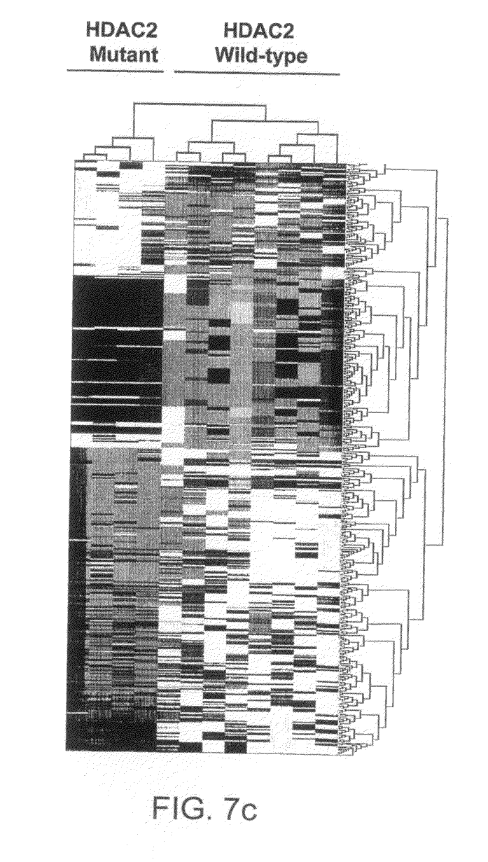

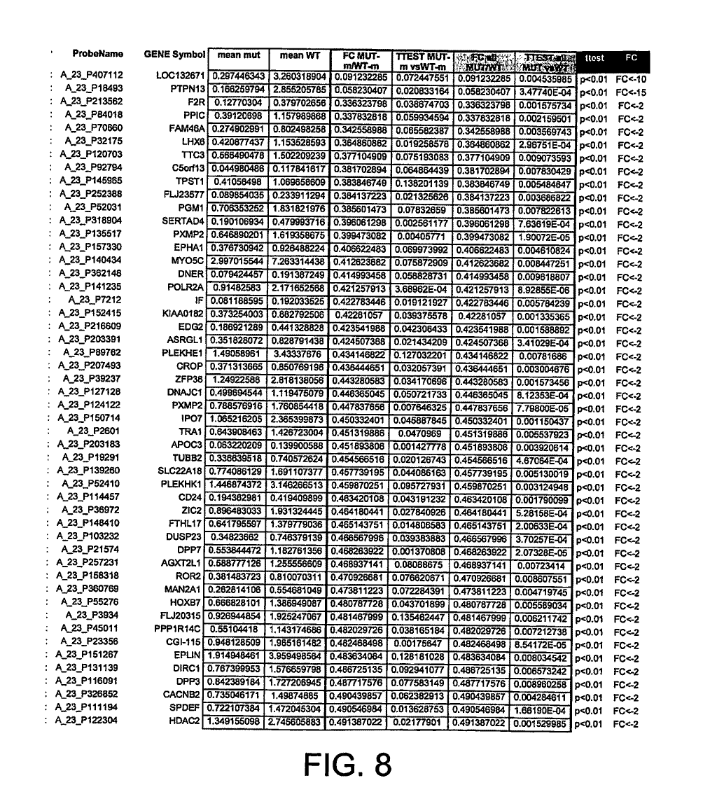

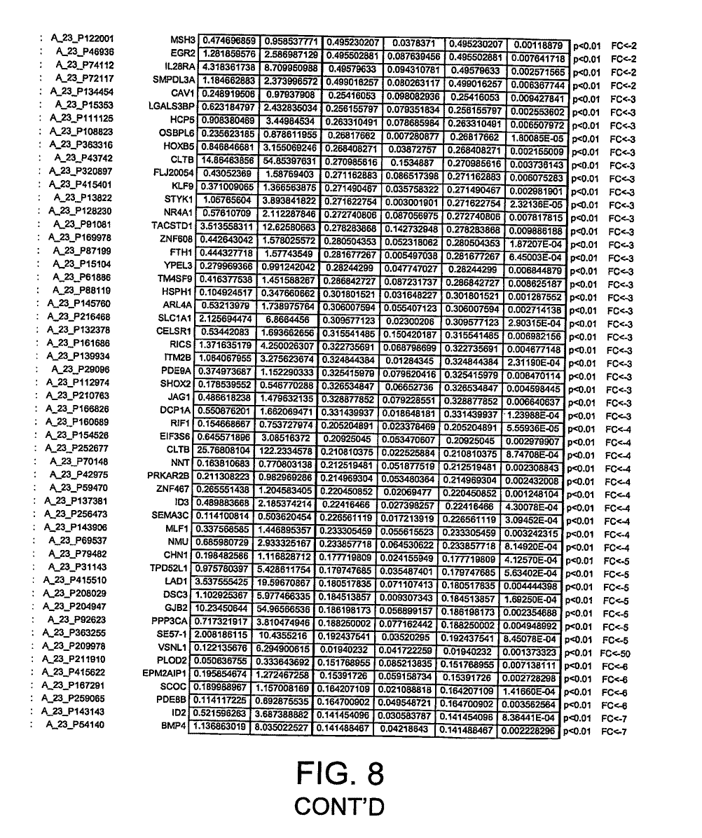

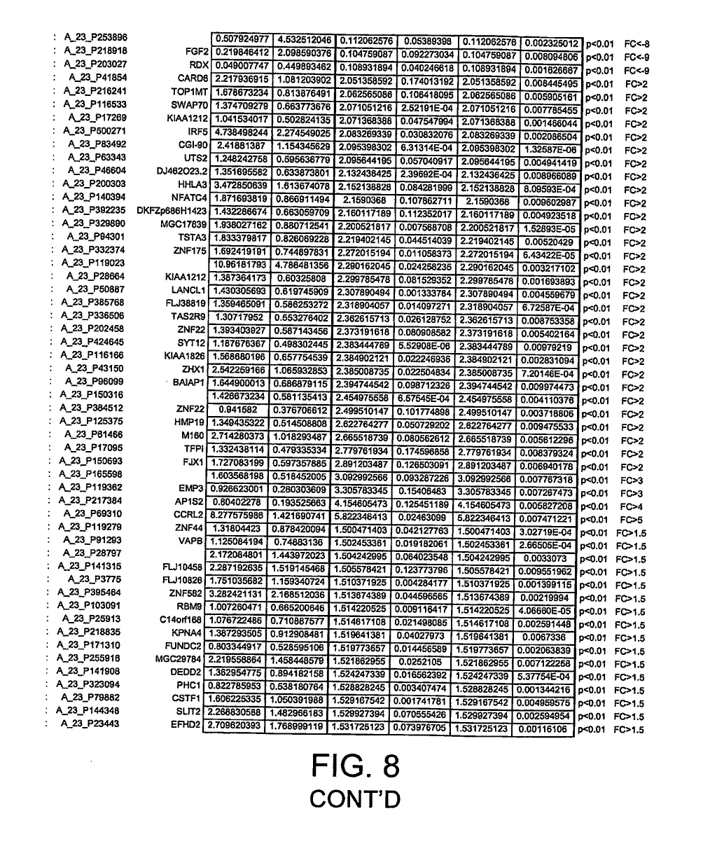

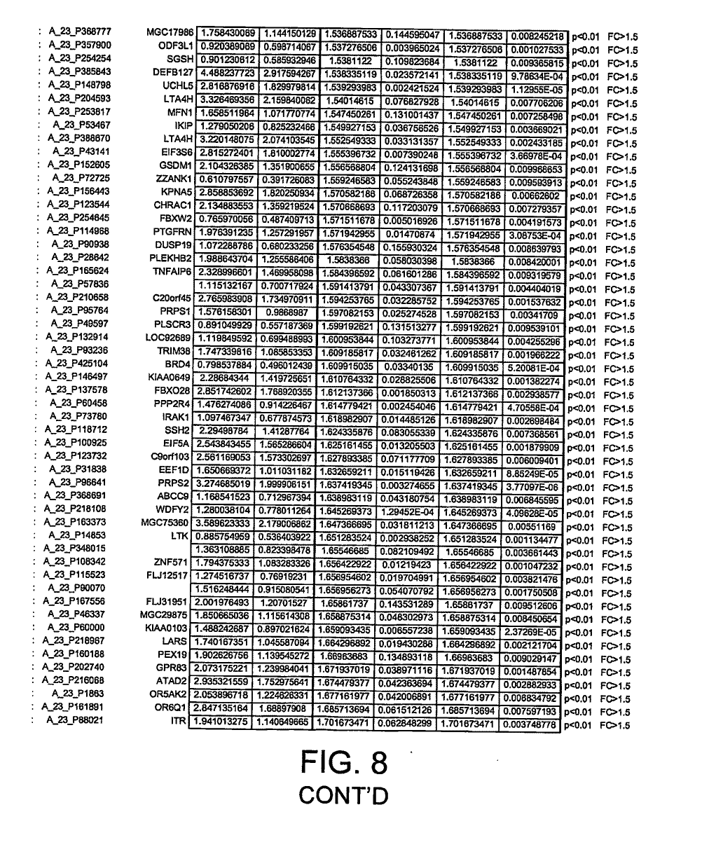

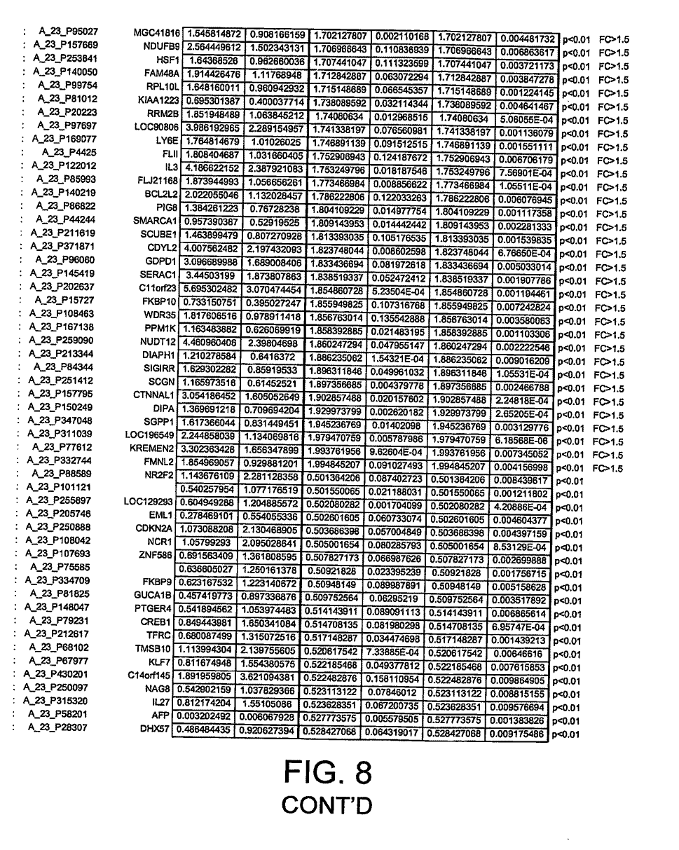

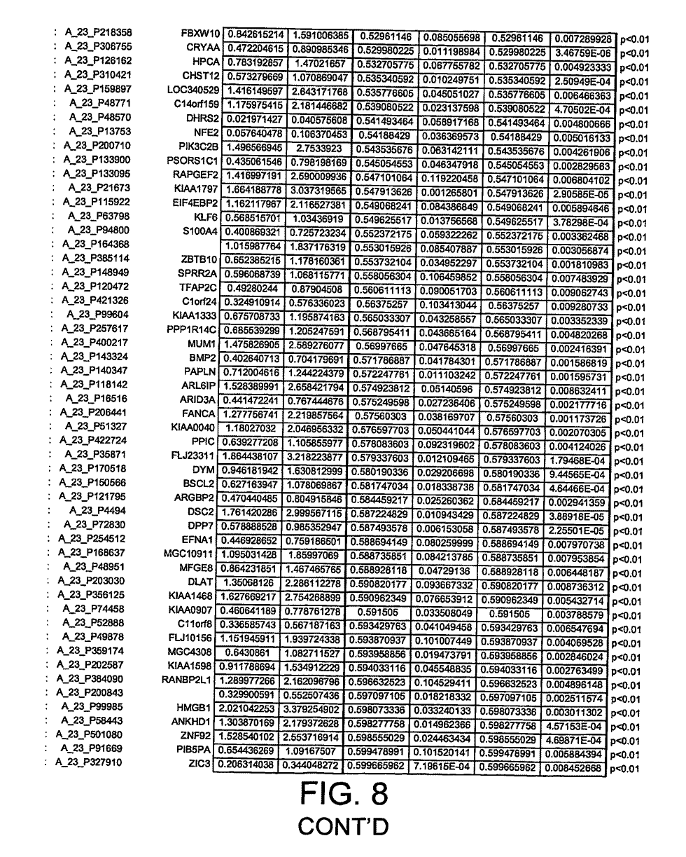

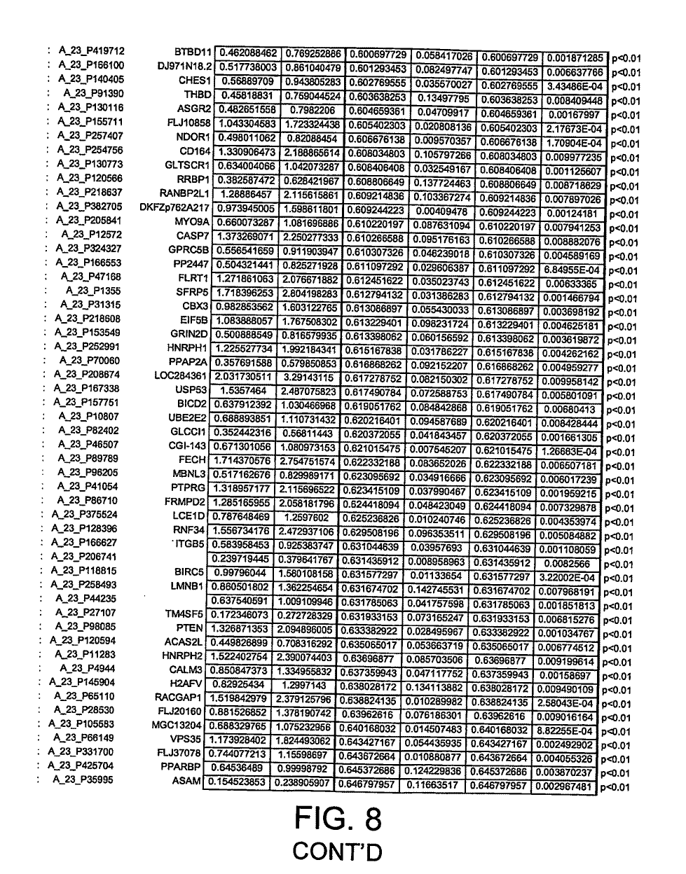

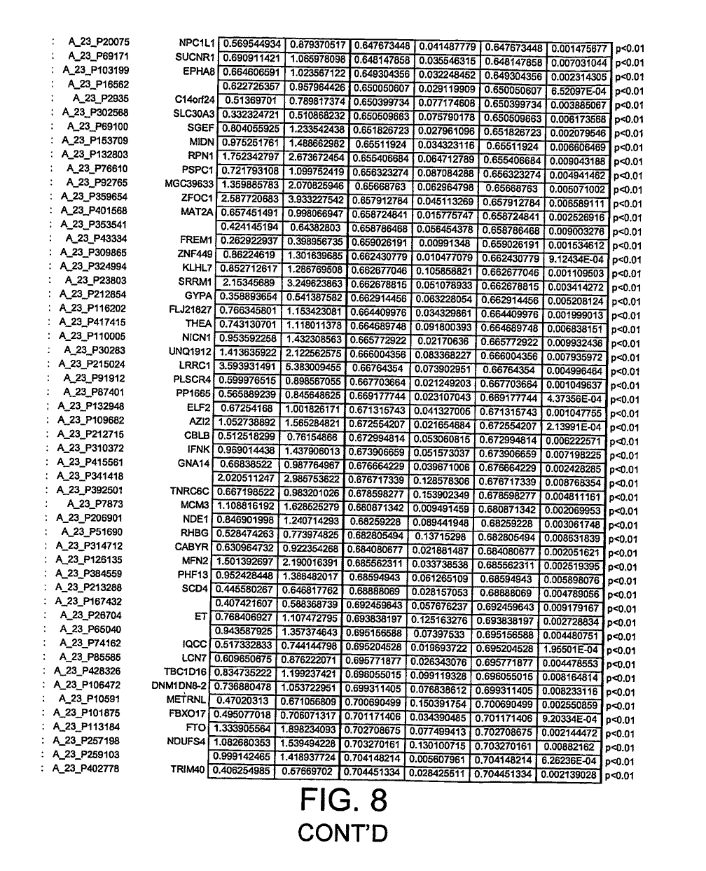

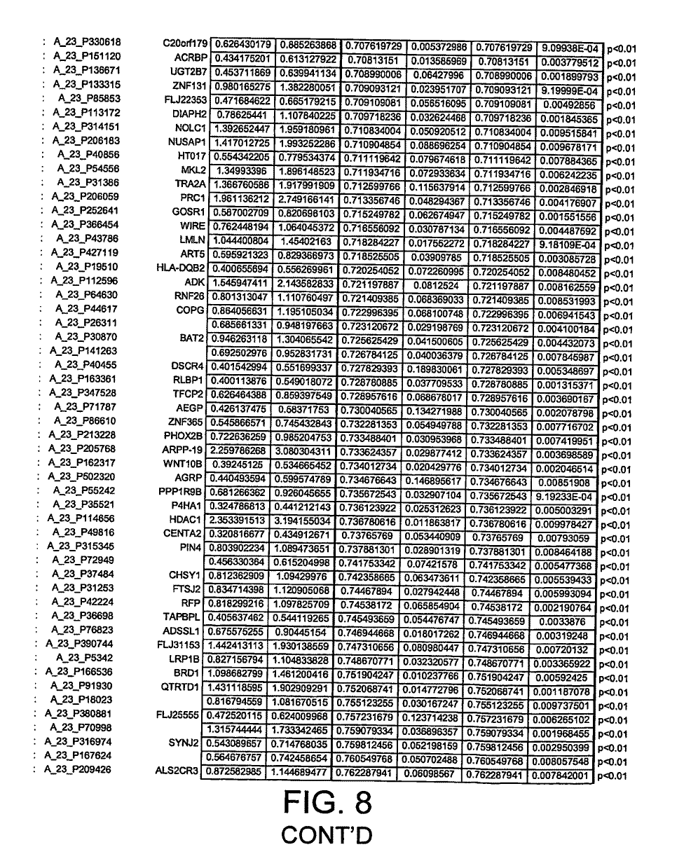

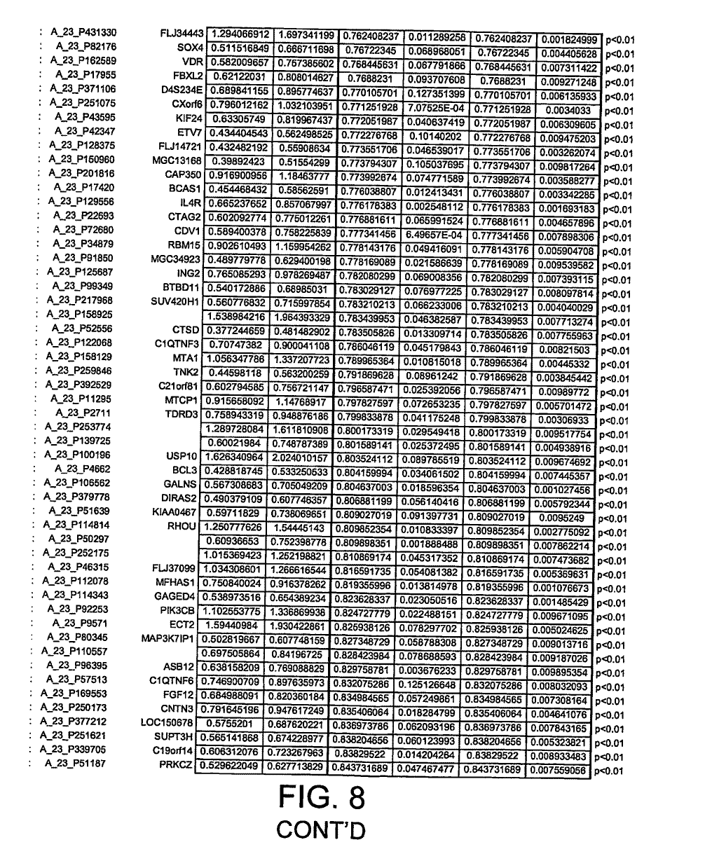

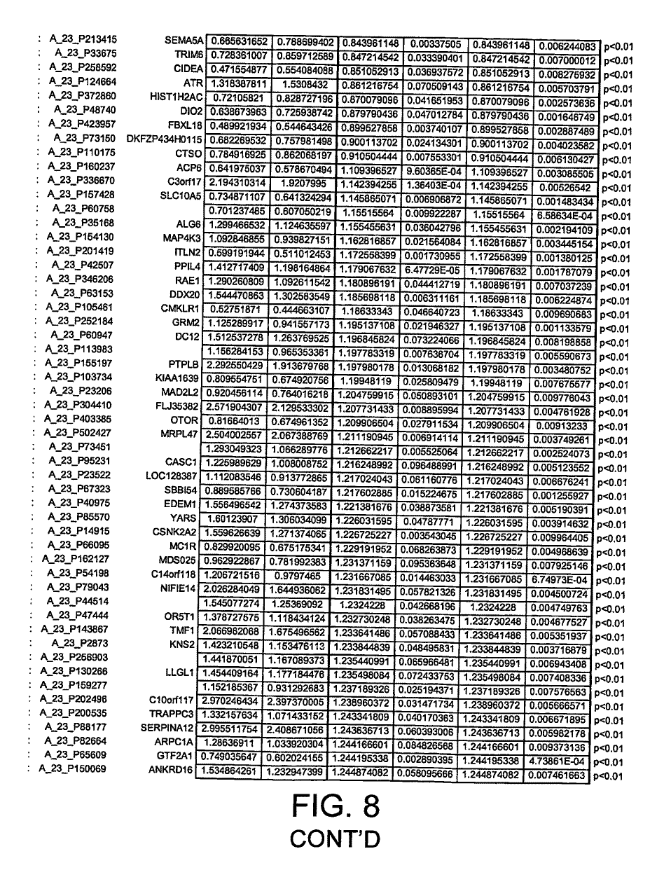

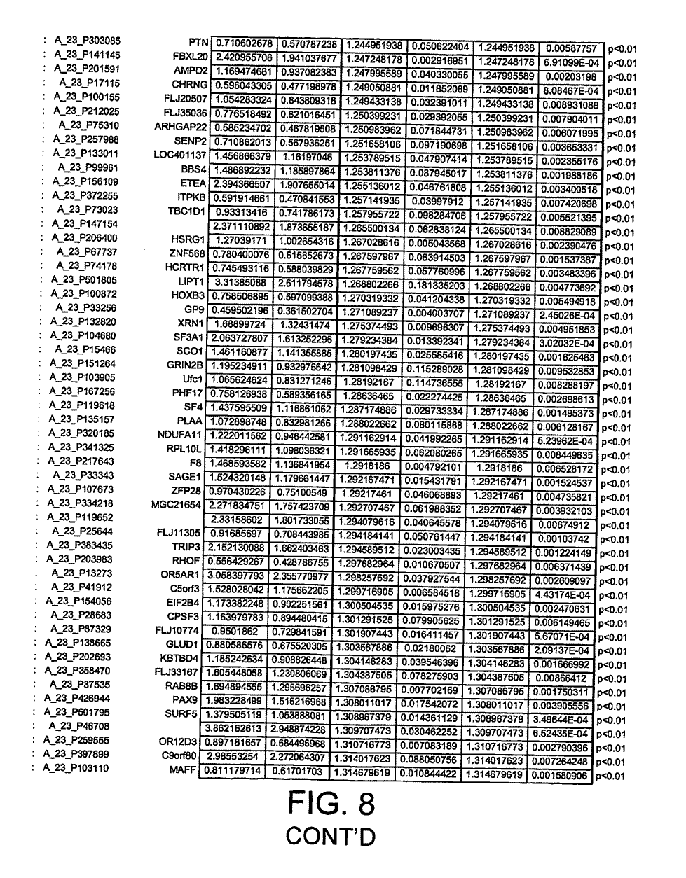

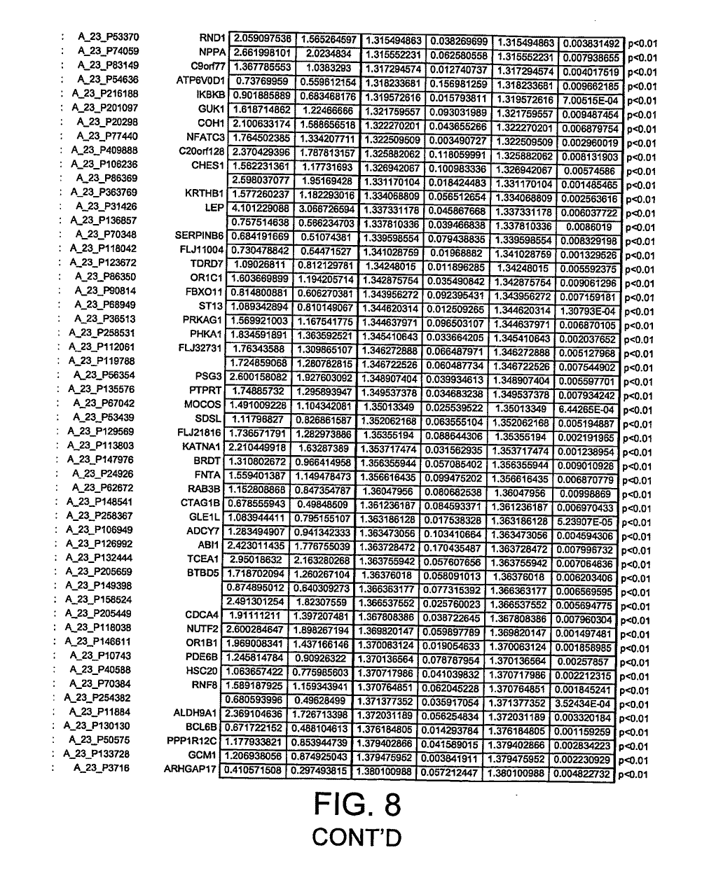

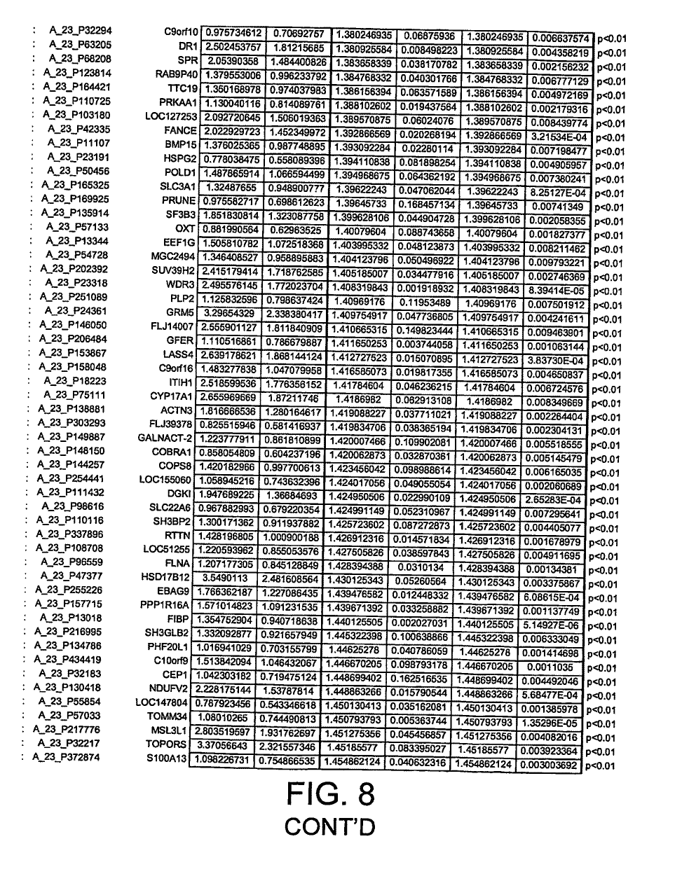

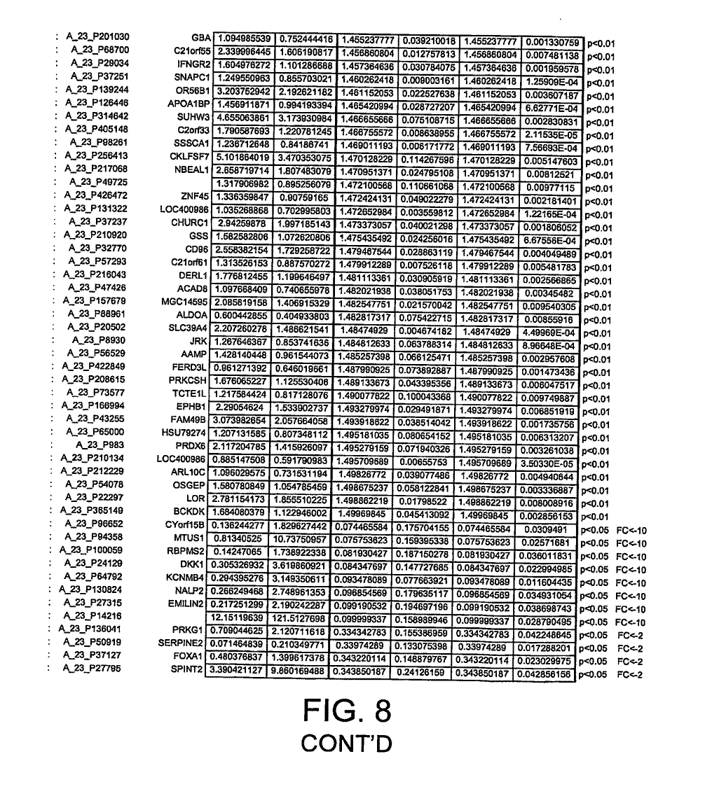

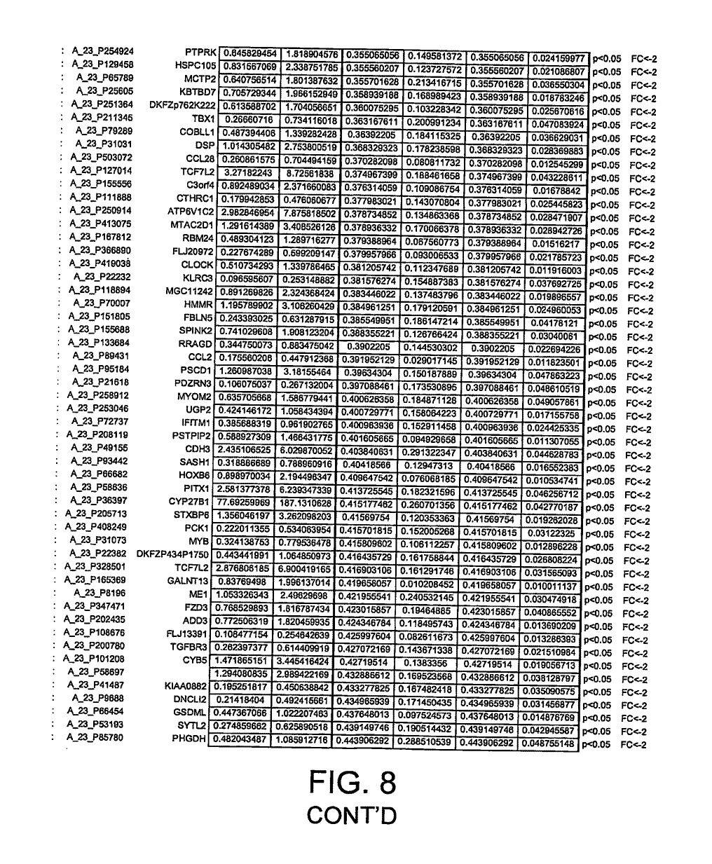

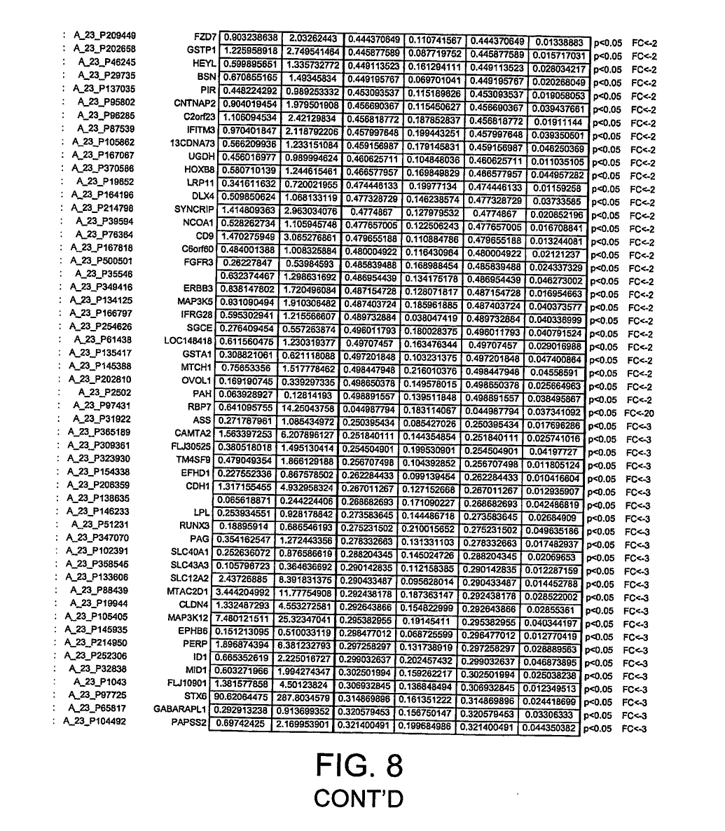

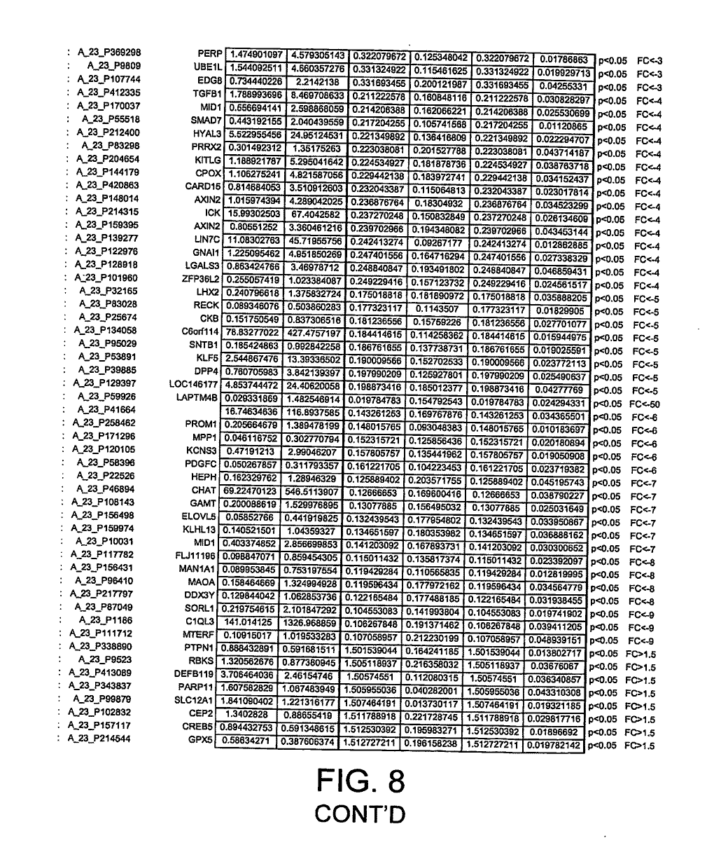

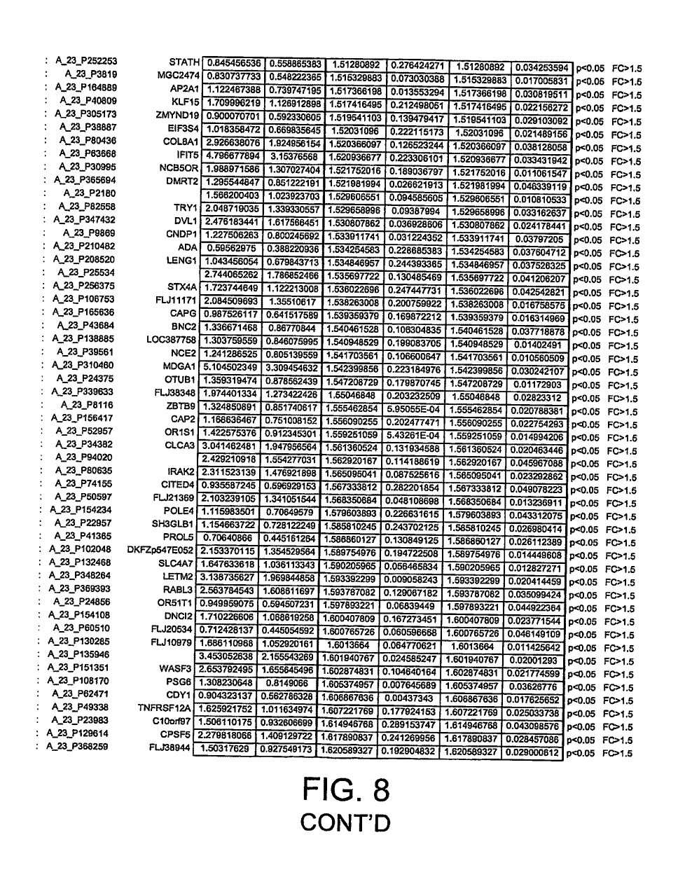

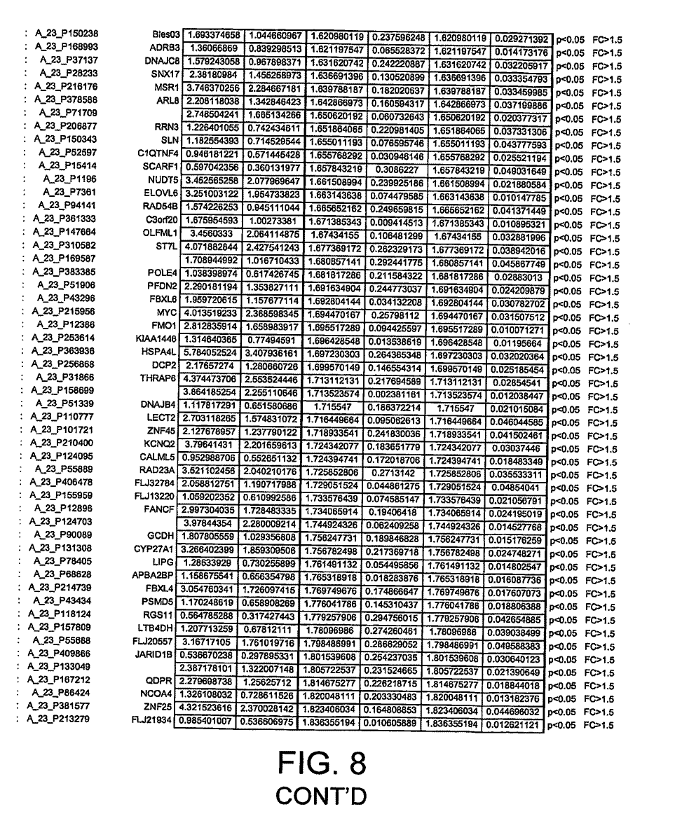

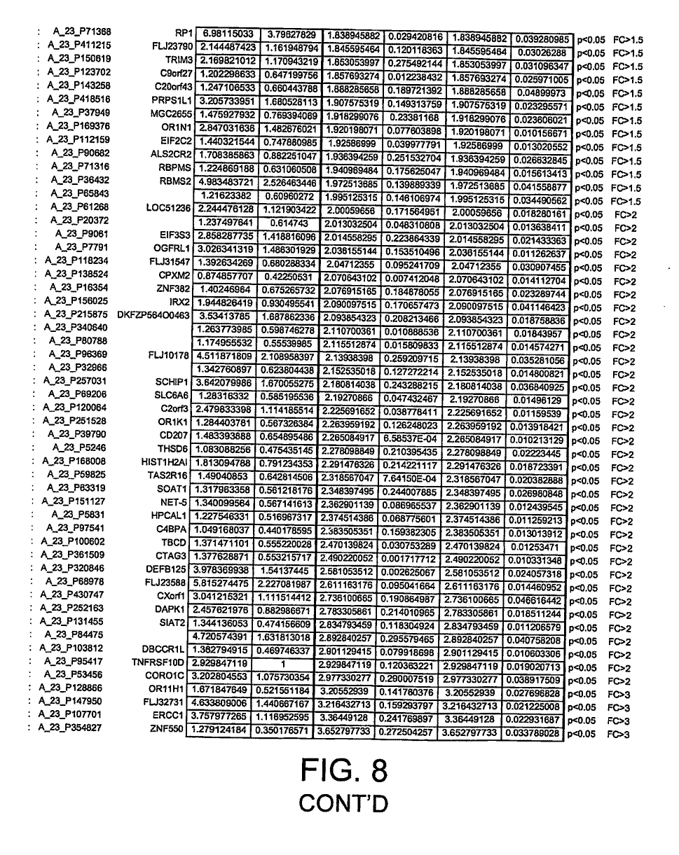

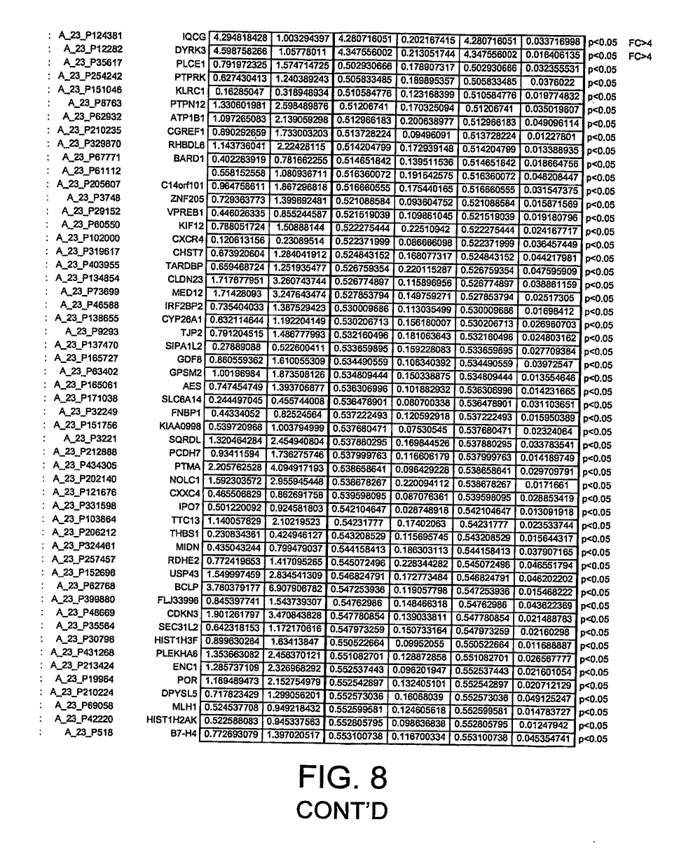

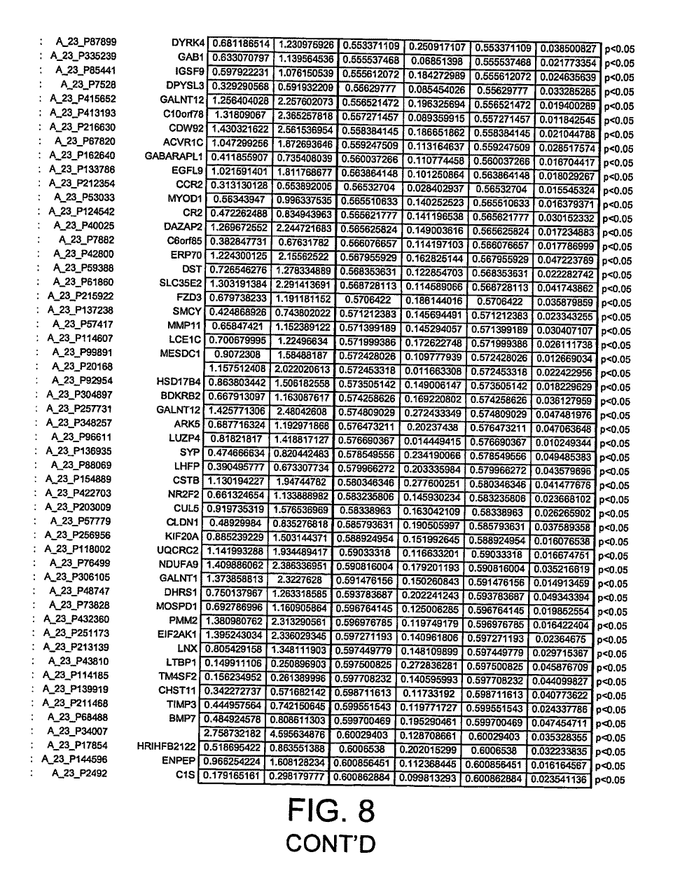

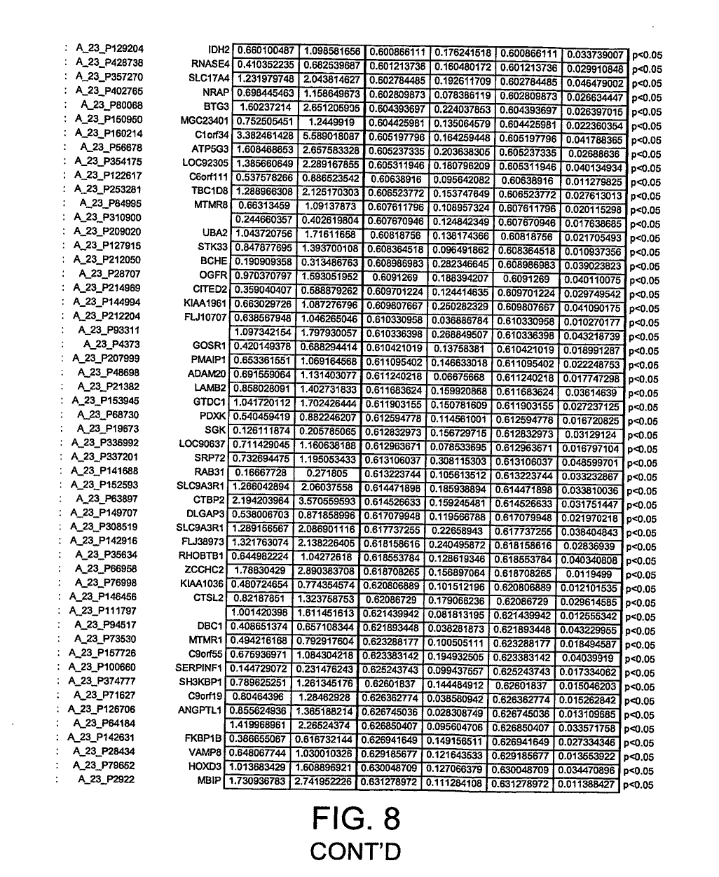

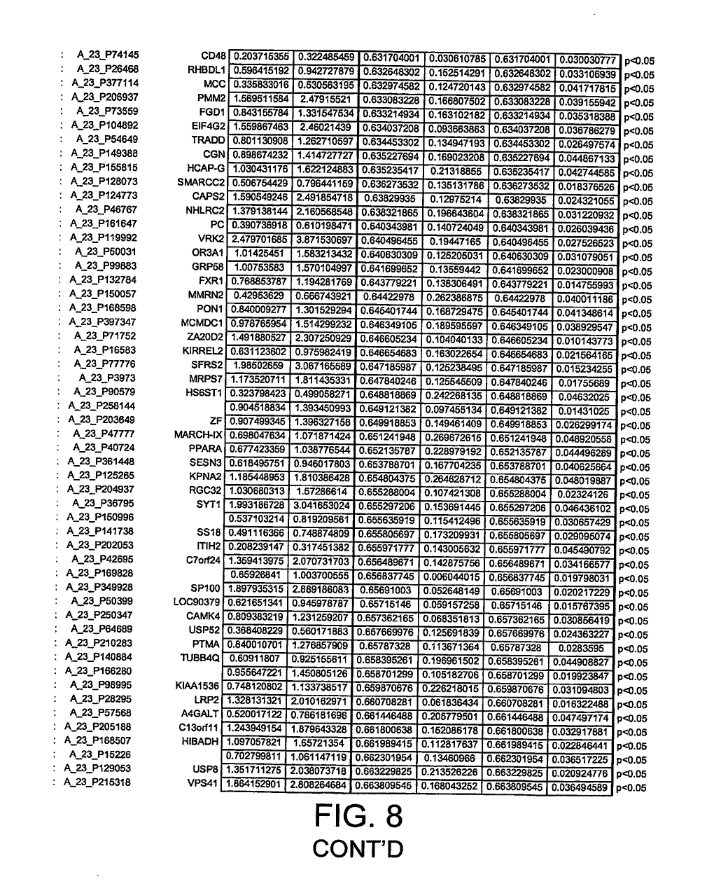

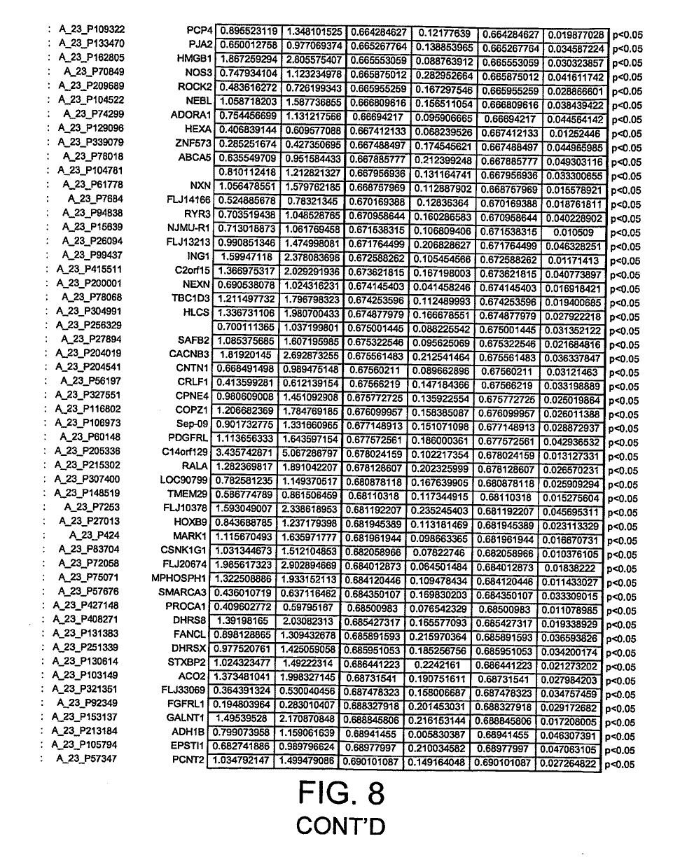

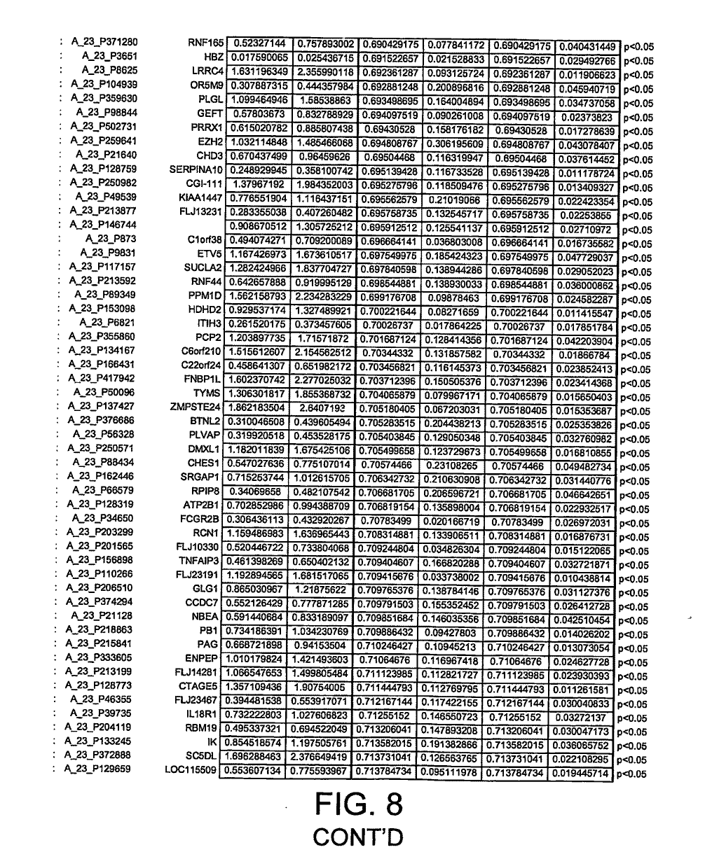

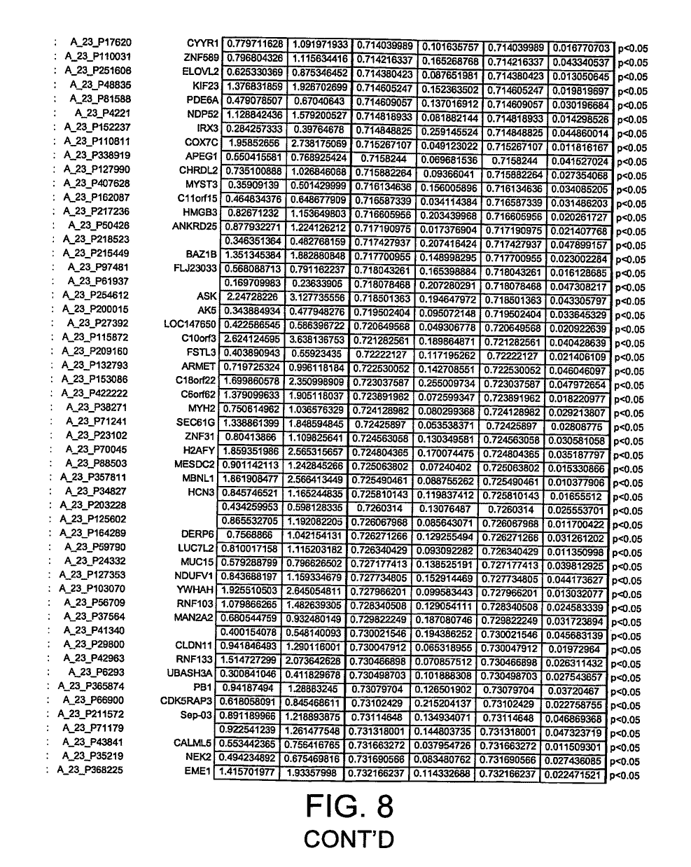

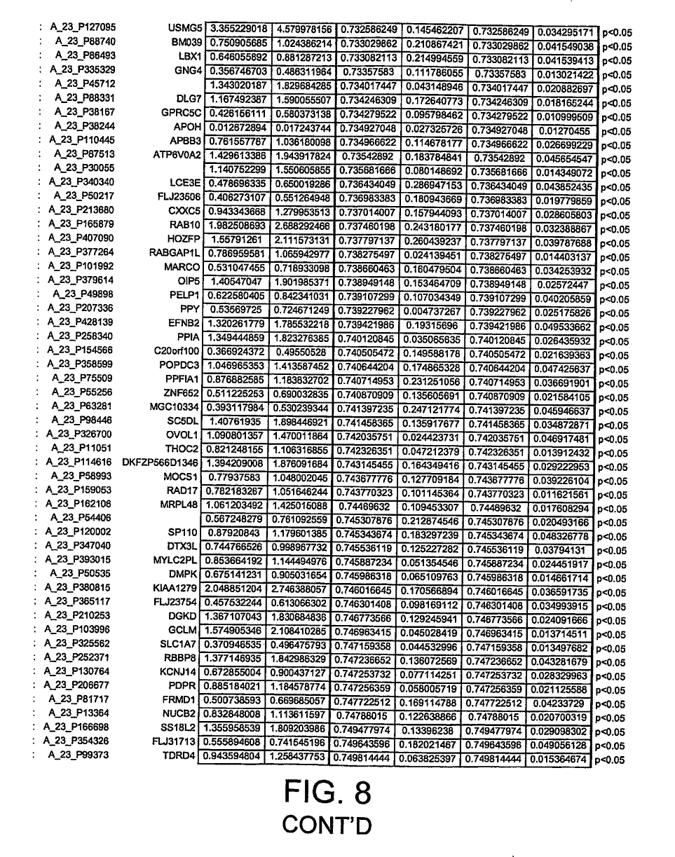

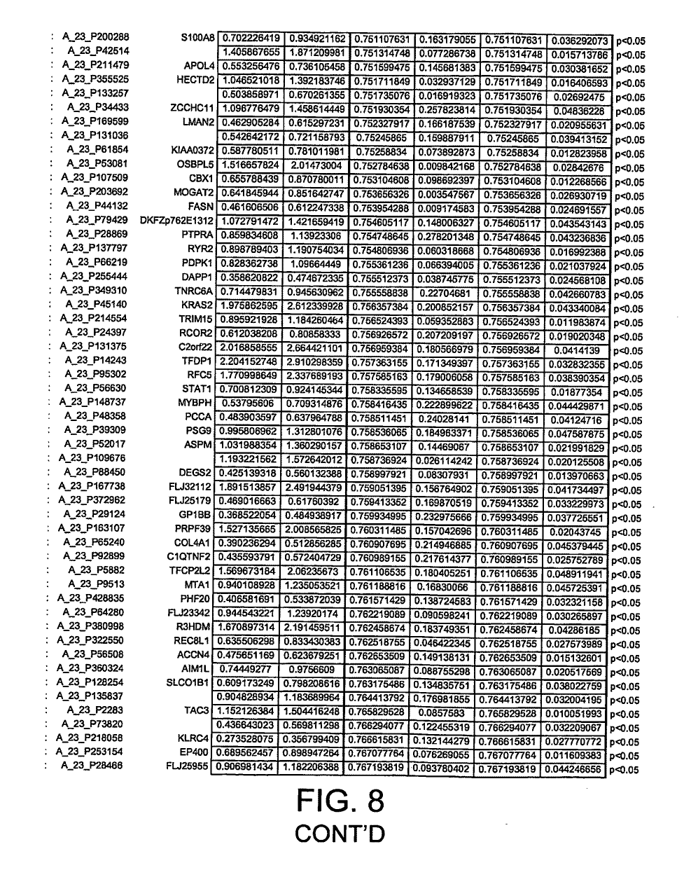

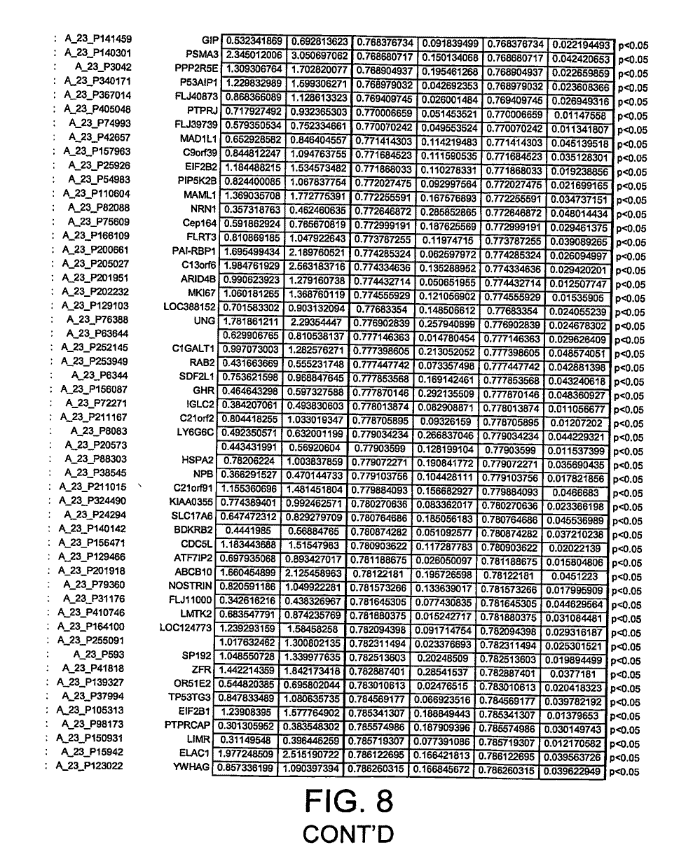

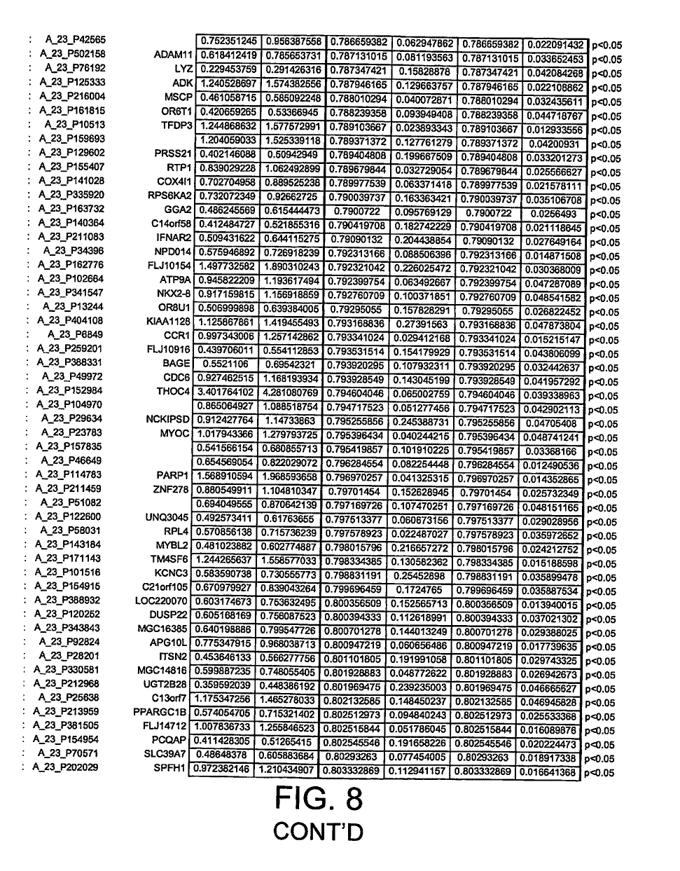

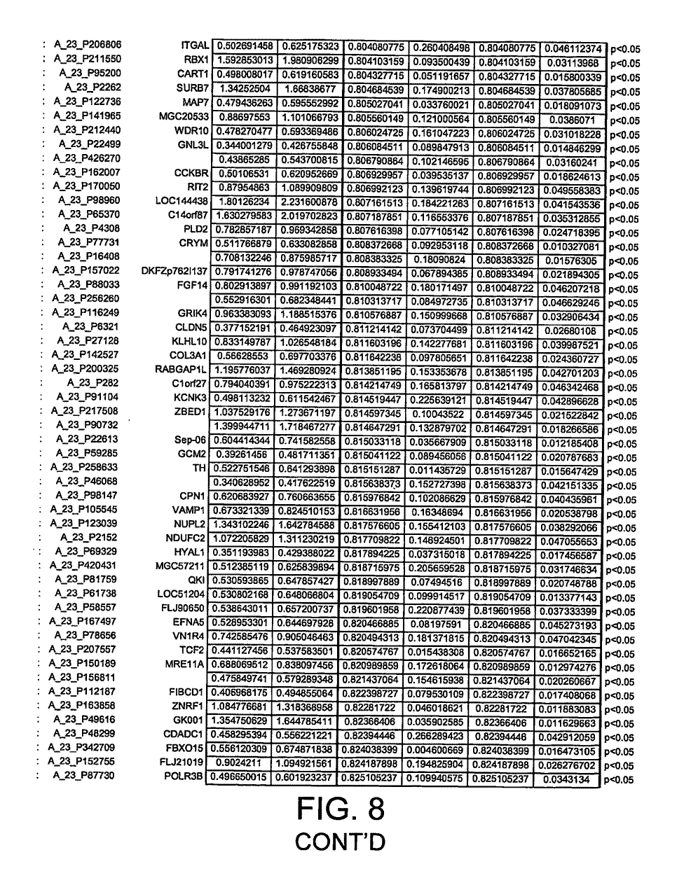

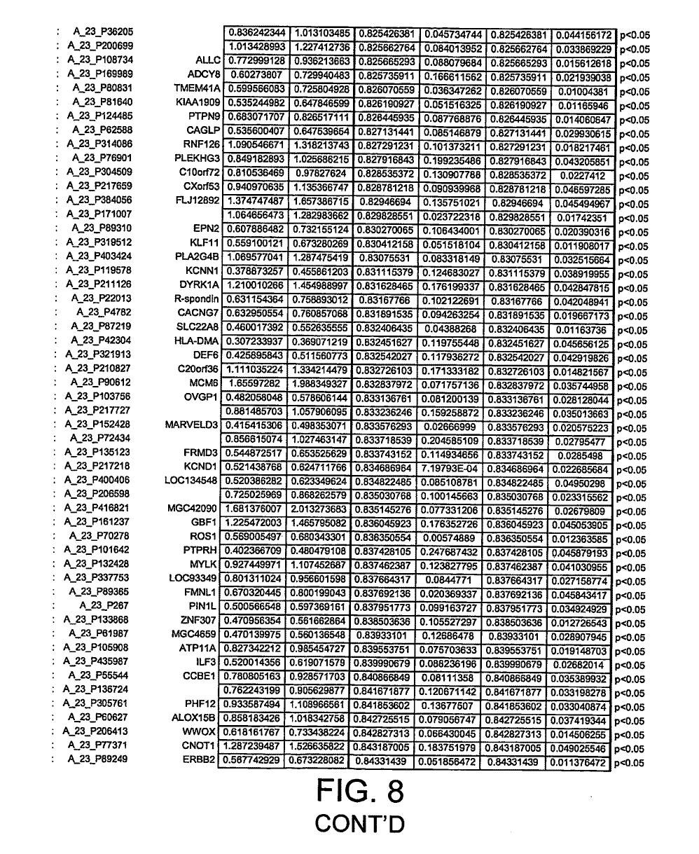

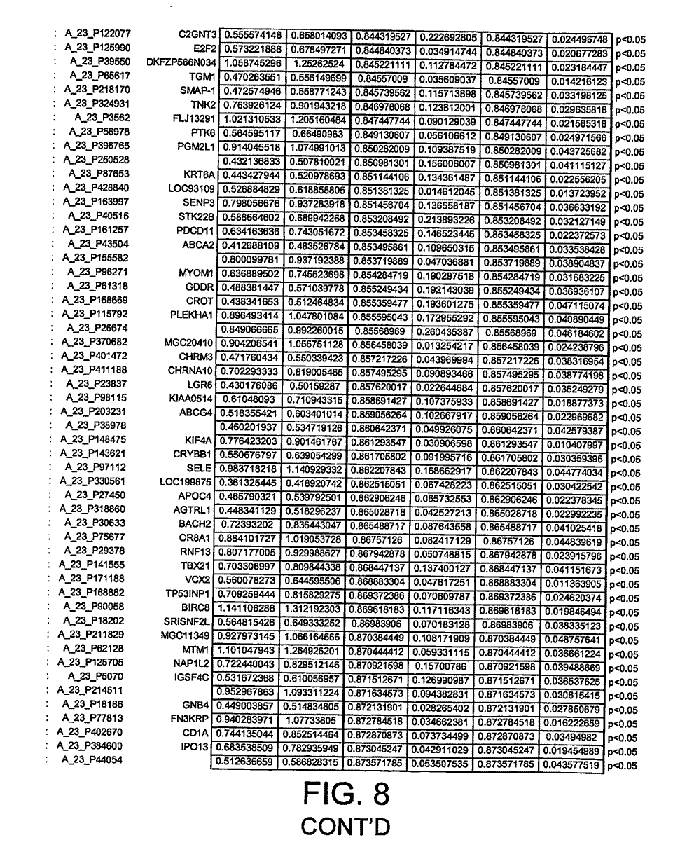

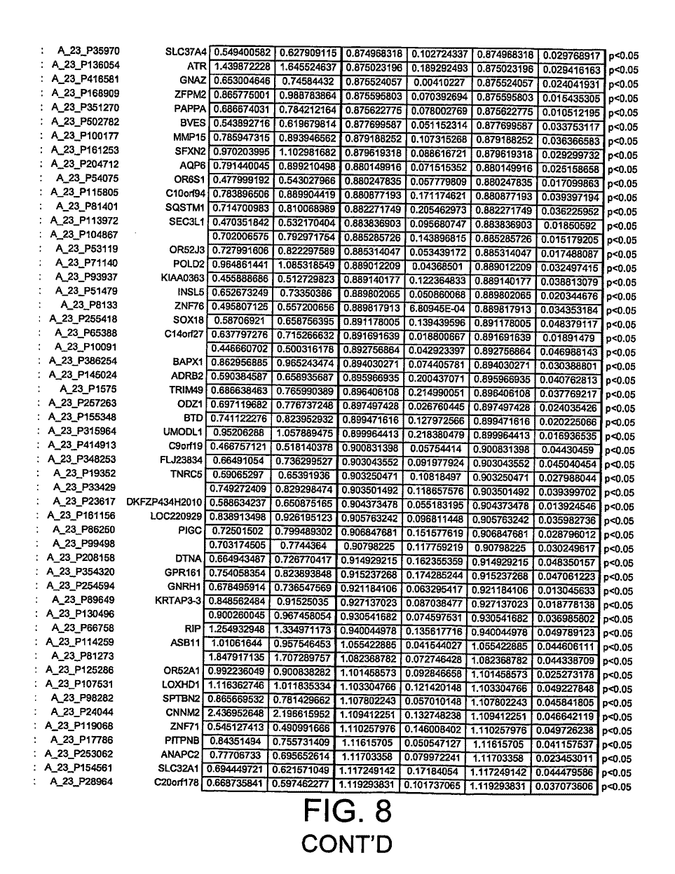

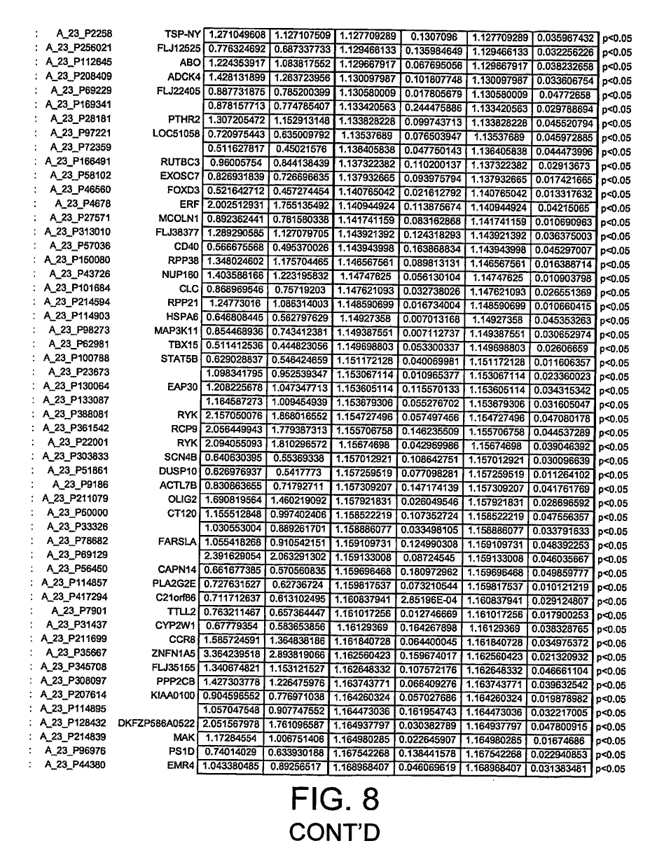

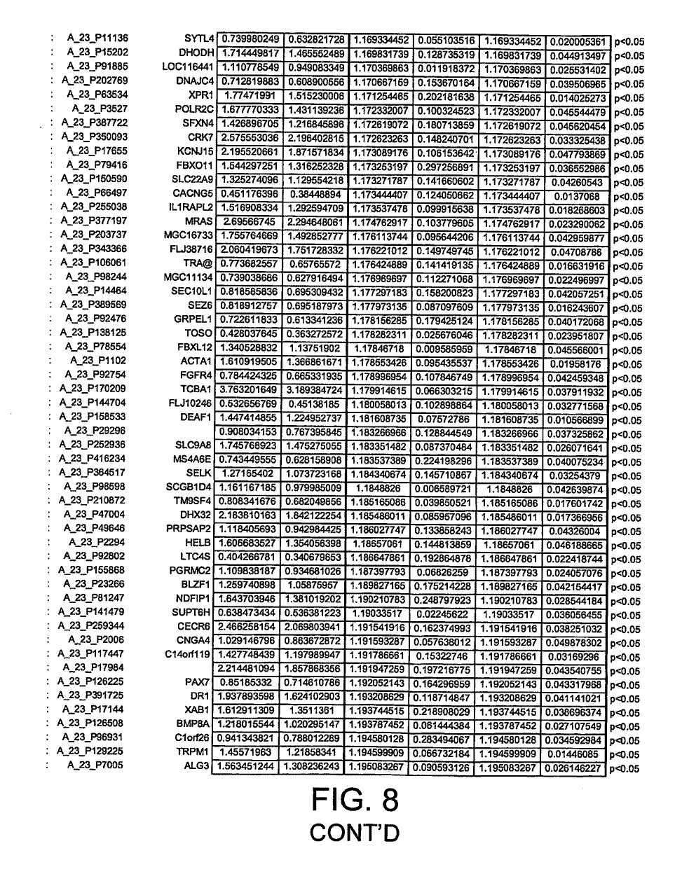

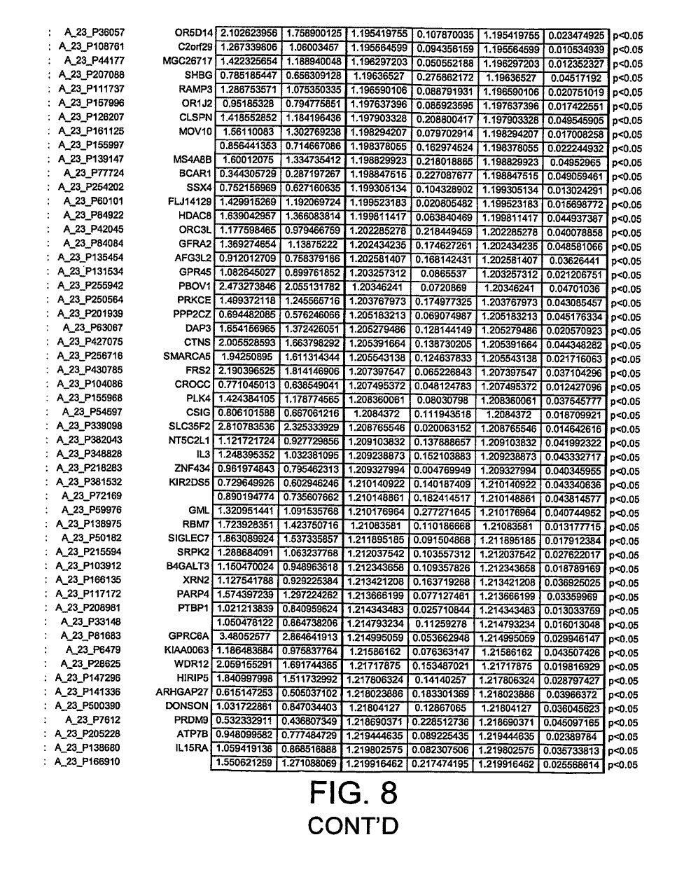

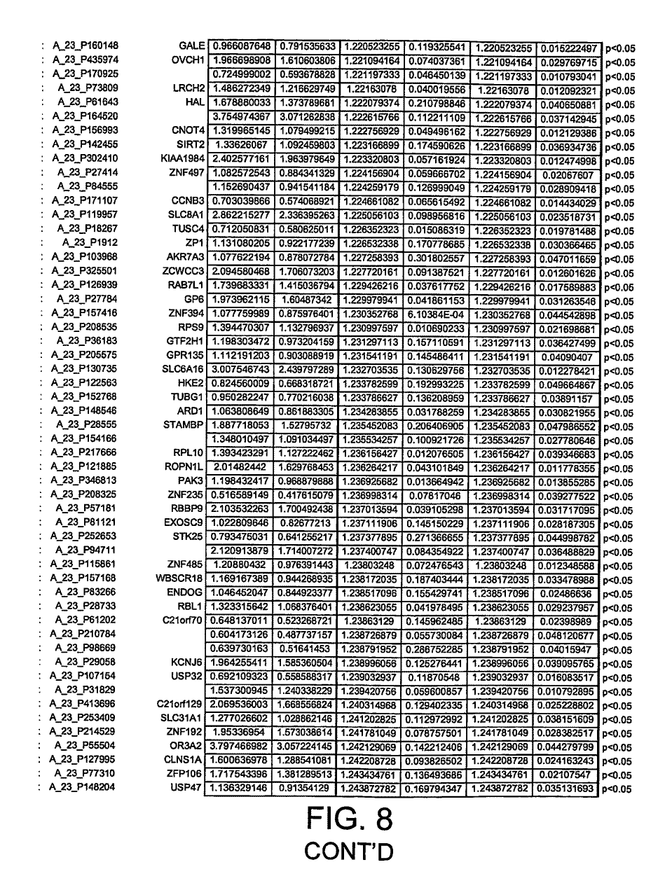

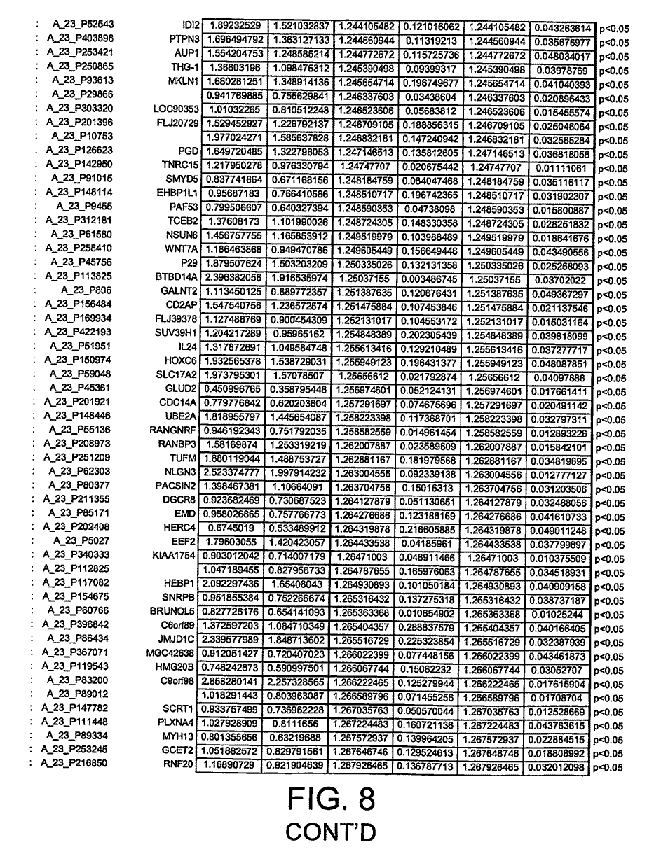

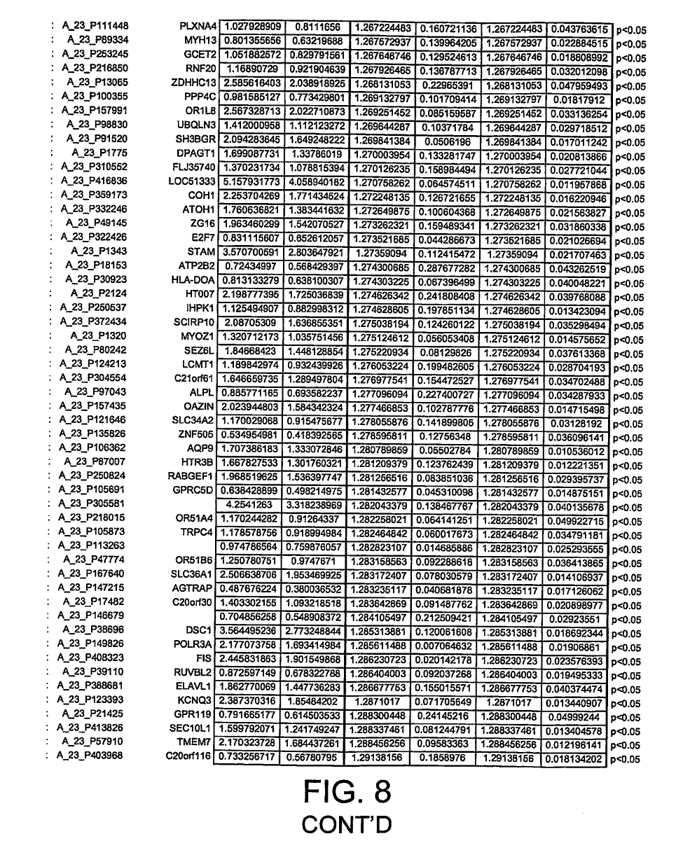

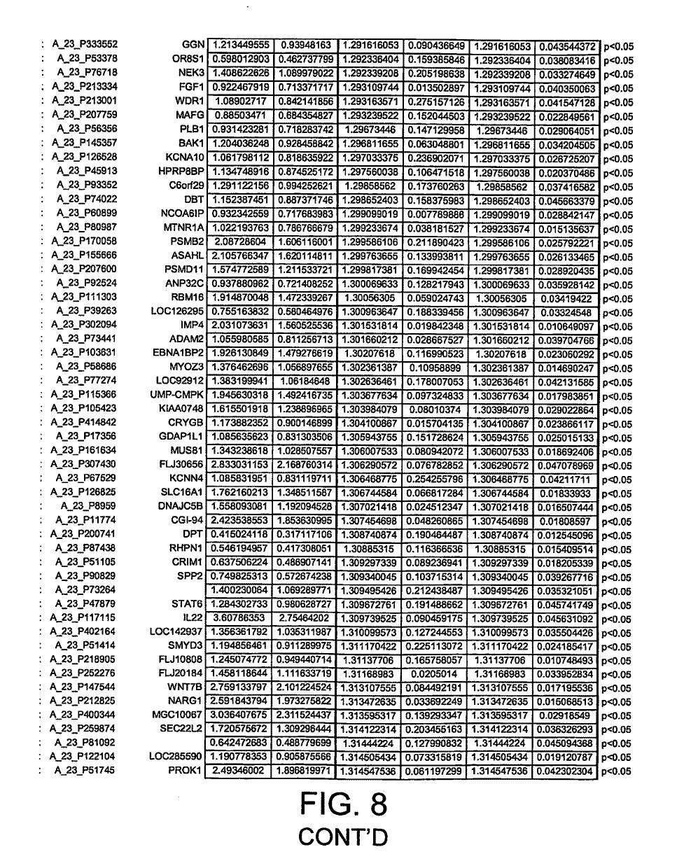

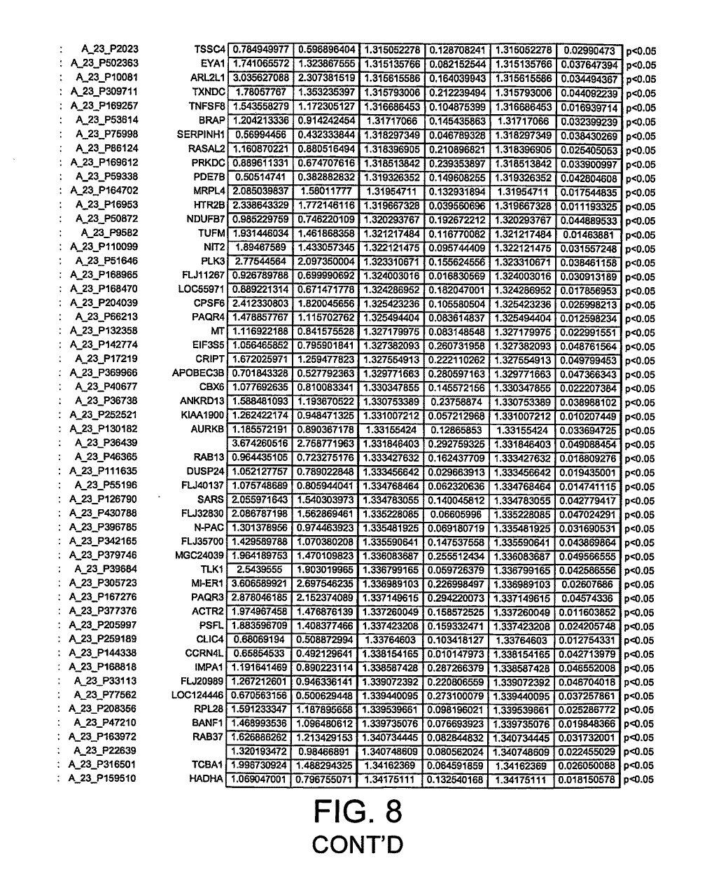

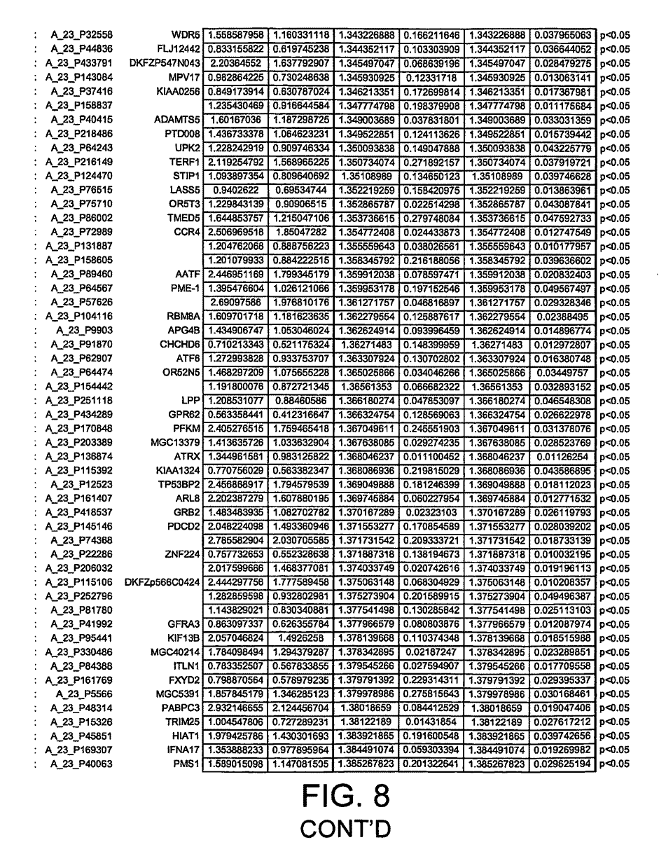

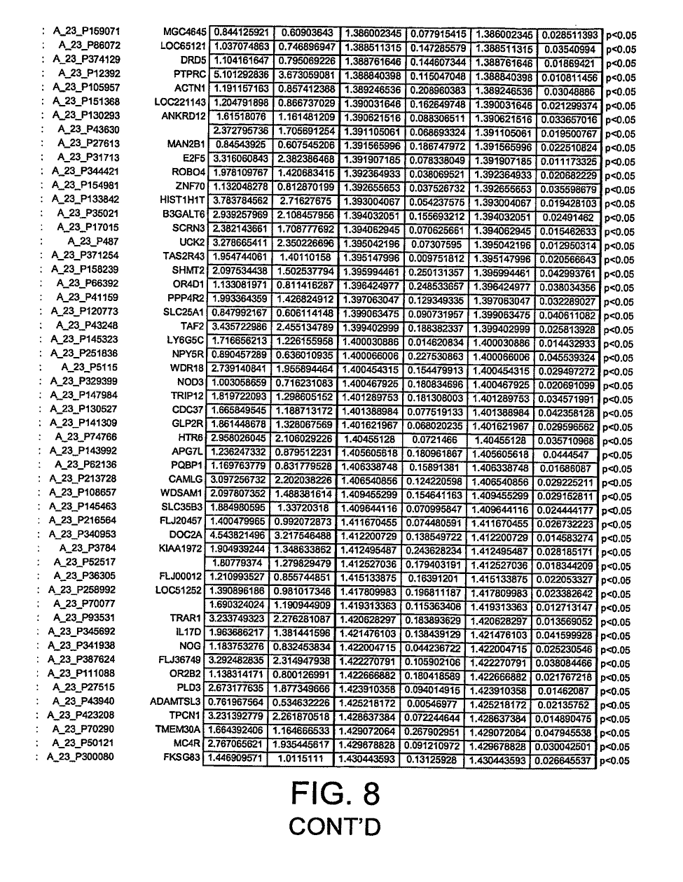

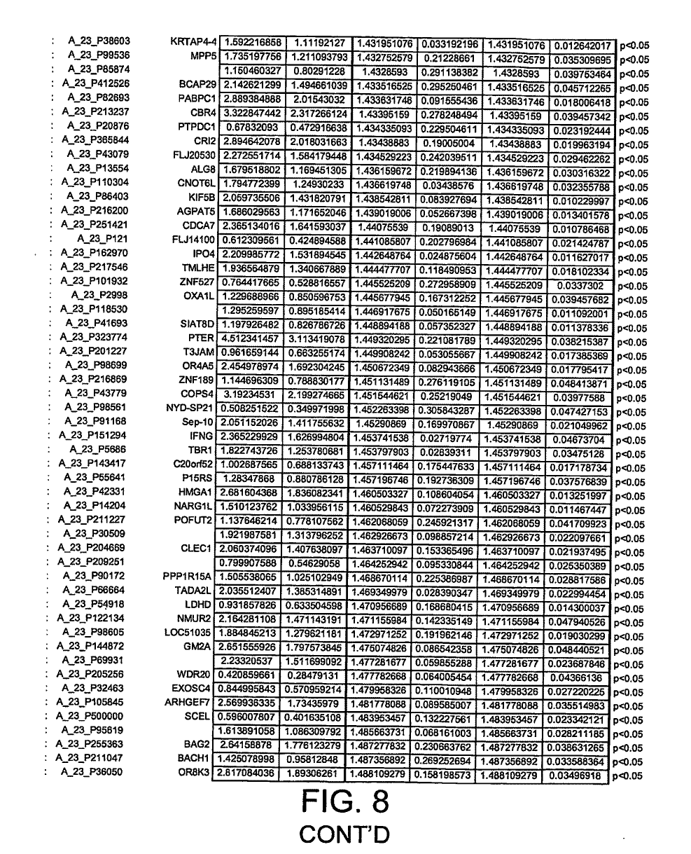

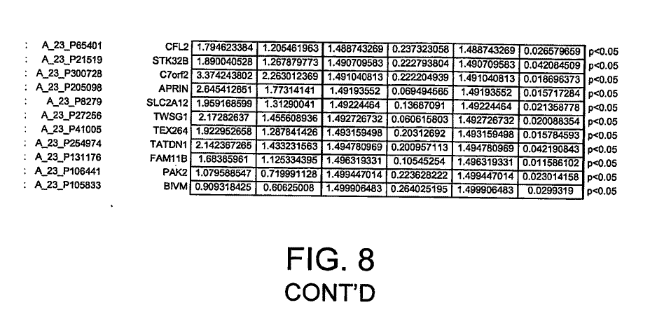

We first screened six colorectal (RKO, SW48, LoVo, HCT-15, Co115 and HCT-116) and four endometrial (AN3CA, SKUT-1, SKUT-1B and HEC1B) cancer cell lines with MSI for the presence of mutations in all the exonic mononucleotide repeats present in the coding sequences of histone deacetylases (HDAC1 and HDAC2), histone acetyltransferases (pCAF), histone methyltransferases (G9a), DNA methyltransferases (DNMT1 and DNMT3b), and methyl-CpG binding proteins (MBD1, MBD2 and MeCP2). The location of the corresponding repeats and the PCR primers used are shown in Supplementary Methods. Only the wild-type sequences were detected for all the genes described, with the single important exception of HDAC2 (FIGS. 1a,b).

We found a frameshift mutation in the HDAC2 gene in the (A)₉ coding microsatellite repeat of exon 1, consisting of the deletion of an A, in two colorectal cell lines (RKO and Co115) and two endometrial cell lines (AN3CA and SKUT-1). We analyzed the RKO and Co115 cell lines and found no evidence of the HDAC2 protein in nuclear extracts (FIG. 1c) and in the immunofluorescence staining (FIG. 1c).

Most importantly, we demonstrated HDAC2 functional abrogation in RKO cells by showing the lack of histone deacetylase enzymatic activity in HDAC2-immunoprecipitated cell extracts of RKO cells compared with HCT-116, SW48 and LoVo cells, these last three having the wild-type HDAC2 coding repeat (FIG. 1d). Since the two alleles of HDAC2 in RKO and Co115 cells are retained by FISH analysis (FIG. 1i) and only mutant alleles were obtained by the sequencing of multiple clones, these observations imply the biallelic inactivation of HDAC2 by the described mutation. In contrast, the two endometrial cancer cell lines were heterozygous for the HDAC2 mutation and haploinsufficiency for HDAC2 function was observed (FIG. 2a). For all cell lines, no significant differences in the levels of HDAC2 mRNA were observed (FIG. 1c, left) and no evidence of HDAC2 mutations were found in colorectal (SW480, SW620, CaCo2 and COLo250) and endometrial (KLE) cancer cell lines that were not MSI.

Once the presence of an inactivating mutation of HDAC2 in cancer cells had been confirmed, it became very important to establish whether the abolition of HDAC2 function altered the biochemical and cellular effects mediated by the histone deacetylase inhibitors (HDACis). This could be a potentially relevant clinical issue, since HDACis are drugs that have significant anticancer activities at doses that are well tolerated by patients in clinical trialsl³. In the case of colorectal cancer, HDACis induce tumor growth inhibition in cell lines^{13,14} and in Apc (min) mice¹⁵. Could the detection of a HDAC2 inactivation mutation in a given tumor predict the response to HDACis? An equivalent pharmacogenetic scenario has been recently presented itself in the presence of somatic mutations in the Epidermal Growth Factor (EGFR) gene in lung neoplasms that render these tumors more sensitive to the killing effect of small-molecule kinase inhibitors of EGFR¹⁶.

To address these issues we assessed the effects of three classical HDACis, the hydroxamic acid trichostatin A and the carboxylic acids butyrate and valproate, on five colorectal cancer cell lines: the HDAC2-mutant cell lines RKO and Co115, and the HDAC2-wild type cell lines HCT-116, SW48 and LoVo. We analyzed the acetylation levels at histones H3 and H4 using western blotting with antibodies raised against tetraacetylated peptides of histones H3 and H43, and high-performance capillary electrophoresis (HPCE)³. We found that whilst butyrate and valproate were able to induce hyperacetylation of histones H3 and H4 in all colorectal cell lines irrespective of their HDAC2 status, trichostatin A was unable to induce the hyperacetylation of either histone in the HDAC2-deficient RKO and Co115 cell lines, but was effective in the HDAC2-wild type cells (FIG. 1e). The HDAC2-deficient cells were resistant to trichostatin A action from a biochemical and also a cellular point of view. We found that valproate and butyrate were able to induce blockage of the cell cycle at G2/M, significantly inhibited proliferation, and induced apoptosis in all cancer cell lines independently of their HDAC2 status, while trichostatin A was only able to accomplish these effects in the HDAC2-proficient cell lines; RKO remained highly resistant (FIG. 1f and FIG. 3c).

We reproduced these results in xenografted nude mice models: butyrate induced tumor shrinkage in all cancer cell lines independently of their HDAC2 status, whereas trichostatin A was only able to accomplish these effects in the HDAC2-proficient cell line (HCT-116), whilst RKO and Co115 remained highly resistant (FIG. 1f). Interestingly, suberoylanilide hydroxamic acid (SAHA), another HDAC is from the same chemical family as trichostatin A, was also unable to efficiently inhibit cell growth and induce apoptosis in HDAC2-deficient RKO cells (FIG. 5).

To further establish a link between HDAC2 mutations and the phenotypes observed, we reconstituted HDAC2 function in HDAC2-deficient cancer cells (RKO) or knocked-down HDAC2 in HDAC2-proficient cells (HCT-116). Transfection of wild-type HDAC2 in RKO cells restored HDAC2 activity (FIG. 6a) and rendered these cells more sensitive to the actions of trichostatin A (FIG. 6b). Most interestingly, the ectopic expression of HDAC2 in these HDAC2-deficient cells induced tumor suppressor-like features, such as growth inhibition in xenografted nude mice and reduced colony formation (FIGS. 6c and 6d). In sharp contrast, the downregulation of HDAC2 by RNA interference in HDAC2 wild-type cells (HCT-116) was associated with a resistance to the effects of trichostatin A and an enhanced tumor growth in the xenograft nude model (FIGS. 6e and 6f).

The involvement of HDAC2 in human cancer was reinforced in an additional experiment. Since HDAC2 seems to be a central player in the epigenetics network at the level of regulation of gene transcription, we wondered whether HDAC2 truncating mutations conferred a particular expression signature to the cancer cells harboring this alteration. Using expression microarray analysis, we found that in unsupervised clustering analysis, the MSI+HDAC2-mutant cell lines (RKO and Co115) grouped together in a separate branch to the MSI+HDAC2-wild-type cell lines (HCT-116, SW48, HCT-15, LoVo) (FIG. 1g). HDAC2-mutant cells are characterized by the overexpression of many tumor-promoting and oncogenic genes, in association with the loss of HDAC2 recruitment to those particular promoters, as determined by chromatin immunoprecipitation (FIGS. 1g, 7a and 7b).

Finally, once we had demonstrated the functional molecular and cellular consequences of harboring an inactivating mutation of HDAC2 in cancer cells, we sought to measure the frequency of the described HDAC2 disruption in human primary tumors. We assessed the HDAC2 mutational status of 228 human primary malignancies with microsatellite instability, including colorectal tumors from HNPCC patients (n=47) and sporadic colorectal (n=127), gastric (n=38) and endometrial (n=16) neoplasms (Table 1). We found that the frameshift mutation in the HDAC2 gene in the (A)₉ coding microsatellite repeat of exon 1 was present in 21% (48 of 228) of the primary tumors analyzed. No significant differences in HDAC2 mutation frequency were found between inherited and sporadic tumors or between tumor types (Table 1).

TABLE 1
Frequency of HDAC2 mutations in cancer cell lines,
primary tumors and normal tissues.
Frequency of HDAC2 mutation in human samples

| Sample type | Cell lines | Tissue samples |
|---|---|---|
| Colon Tumors from HPNCC | — | 8/47 (17%) |
| Sporadic Colon Tumors MSI+ | 2/6 | 26/127 (20.4%)^{b} |
| Sporadic Gastric Tumors MSI+ | — | 11/38 (28.9%) |
| Sporadic Endometrial Tumors | 2/4 (50%)^{c} | 3/16 (18.7%) |
| Sporadic Colon Tumors MSI− | 0/4 | 0/40 |
| Normal Colon | — | 0/50 |
| Normal Lvmphocvtes | — | 0/50 |

^{a}Homozygous mutations.
^{b}For six cases analyzed, five homozygous and one heterozygous mutations were observed.
^{c}Heterozygous mutations

For 17 cases of sporadic colon tumors with microsatellite instability, we conducted a double-blind immunohistochemical analysis of HDAC2 mutational status (FIG. 2). In all cases with the wild-type HDAC2 gene (n=11), HDAC2 protein was strongly expressed. In contrast, of the six HDAC2 mutant tumors, five (83%) completely lacked HDAC2 expression, whilst a heterogeneous pattern of loss of expression was observed in the remaining case. Laser microdissection analysis of the HDAC2-stained sections confirmed that the presence of the HDAC2 mutation was always associated with the loss of HDAC2 signal. The described HDAC2 mutation was not present in primary colorectal tumors without microsatellite instability (0/40), normal colorectal mucosa (0/50) or in normal lymphocytes from healthy donors (0/50) (Table 1, supra).

Furthermore, similar to what we observed in the cancer cell lines, an expression microarray analysis of twelve MSI+primary colorectal tumors, eight wild-type and four HDAC2 mutant, clustered in a separate branch to the HDCA2-mutant samples, underscoring the particular tumor phenotype associated with the mutation (FIG. 7c and the data produced from the gene expression analysis is reproduced in full for those genes whose expression was significantly altered between tumors and wild type cells in the table in FIG. 8).

### REFERENCES

1. Jones, P. A. & Baylin, S. B. Nat. Rev. Genet. 3, 415-428 (2002).
2. Feinberg, A. P. & Tycko, B. Nat. Rev. Cancer 4, 143-153 (2004).
3. Fraga M F, et al. Nat. Genet. 37, 391-400 (2005).
4. Seligson D B, et al. Nature 435, 1262-1266 (2005).
5. Jenuwein, T. & Allis, C. D. Science 293, 1074-1080 (2001).
6. Bannister, A. J. & Kouzarides, T. 376, 269-288 (2004).
7. Gayther, S. A., et al. Nat. Genet. 24, 300-303 (2000).
8. Ionov, Y., Matsui, S.& Cowell, J. K. Proc. Natl. Acad. Sci. USA 101, 1273-1278 (2004).
9. Lynch, H. T. & de la Chapelle, A. N. Engl. J. Med. 348, 919-932 (2003).
10. Herman, J. G., et al. Proc Natl Acad Sci USA 95, 6870-6875 (1998).
11. Markowitz, S., et al. Science 268, 1336-1338 (1995).
12. Rampino, N., et al. Science 275, 967-969 (1997).
13. Marks, P. A. & Jiang, X. Cell Cycle 4, 549-551 (2005).
14. Archer, S. Y., Meng, S., Shei, A. & Hodin, R. A. Proc. Natl. Acad. Sci. USA 95, 6791-6796 (1998).
15. Myzak, M. C., et al., FASEB J. Jan. 11, (2006).
16. Pao, W. & Miller, V. A. J. Clin. Oncol. 23, 2556-2568 (2005).

### 3. Methods

### Cell Lines and Primary Tumor Samples.

Human colorectal and endometrial cancer cell lines were obtained from the American Type Culture Collection. HDAC inhibition treatment was developed adding 0.25 μM trichostatin A, 10 mM sodium valproate or 10 mM sodium butyrate to the culture medium for 24 hours. We obtained DNA samples from 228 human primary malignancies corresponding to colon tumors of HPNCC patients (n=47) and sporadic MSI+colon (n=127), MSI-colon (n=40), gastric (n=38) and endometrial carcinomas (n=16), as well as normal colon tissue (n=6), at the time of clinically indicated surgical procedures. Normal lymphocytes from healthy donors (n=50) was also used. DNA was extracted using standard protocols.

### Mutation Analysis.

Genomic DNA from cell lines and primary tumors and cDNA from the cell lines were amplified by PCR. Direct sequencing of PCR products and recombinant plasmids from ten clones of every sample were sequence in a automatized sequencer ABI Prism 3700. The genes studied, their locations and the primers used are described in the following table:

TABLE 2
Primers used for amplification of various genes.

| | | | Repeat | |
|---|---|---|---|---|
| Gene | Location | Repeat | location | Primers |
| HDAC2 | 6q21 | (A)₉ | Exon 1 | F: 5′-ACCTCCGATTCCGAGCTTT-3′ |
| | | | | R: 5′-ACCTCCGATTCCGAGCTTT-3′ |
| DNMT3B | 20q11.2 | (G)₆ | Exon 7 | F: 5′-CAGAGCAGCAGTACCCCCTA-3′ |
| | | | | R: 5′-CCTCTCGGCCATACCTGATA-3′ |
| DNMT1 | 19p13.2 | (A)₇ | Exon 24 | F: 5′TGACGATGAGGAAGTCGATG-3′ |
| | | | | R: 5′-CTTCTCCGACCCAAGAGATG-3′ |
| DNMT1 | 19p13.2 | (G)₆ | Exon 32 | F: 5′-CGAGCGAGCCAGAGATAGAG-3′ |
| | | | | R: 5′-GGGCTCACCAGGTATTCAGA-3′ |
| HDAC1 | 1p34 | (G)₆ | Exon 12 | F: 5′-ACGAATTGCCTGTGAGGAAG-3′ |
| | | | | R: 5′-TGGGTCTTTCTCTTTTTCATCC-3′ |
| MBD1 | 18q21 | (A)₆ | Exon 16 | F: 5′-GACCTGCTCCCTTTTCCCTA-3′ |
| | | | | R: 5′-AGCCAGTCTGCAAATATCACC-3′ |
| MBD2 | 18q21 | (C)₇ | Exon 1 | F: 5′-AGCGGGAAGAGGATGGATT-3′ |
| | | | | R: 5′-CCCAGGGAGGTACCTGAAGT-3 |
| MeCP2 | Xq2a | (G)₆ | Exon 5 | F: 5′-TGAAAAGGGTCCTGGAGAAA-3′ |
| | | | | R: 5′-CGTTTGATCACCATGACCTG-3 |
| MeCP2 | Xq28 | (C)₆ | Exon 6 | F: 5′-ACCACTCAGAGTCCCCAAAG-3′ |
| | | | | R: 5′-TCTGGGCATCTTCTCCTCTT-3′ |
| PCAF | 3p24 | (C)₆ | Exon 2 | F: 5′-CCATTTTTAGGCCGAGGAGT-3′ |
| | | | | R: 5′-ATGGCTACAACTCCGACAGG-3′ |
| G9a | 9q34.3 | (C)₆ | Exon 3 | F: 5′-CCCAGAGAAGTTCGAGAAGC-3′ |
| | | | | R: 5′-GCCATGTAGCACTGGTTCTG-3′ |
| G9a | 9q34.3 | (A)₆ | Exon 5 | F: 5′-GCTTGCTTGCCTTTTGTTTT-3′ |
| | | | | R: 5′-TCTCAATCACCGTCCTCTGTT-3′ |

### FISH Analysis

Fluorescence in situ hybridization (FISH) was performed by standard methods, which include denaturations steps, overnight hybridization at 37° C., and two washes, one in 0.4×SSC at 75° C. and another in 2×SSC at room temperature. The BAC clone containing the HDAC2 gene (RP11 456N11) was a kind gift from Dr. Mariano Rocci, at the University of Bari (Italy). Control BAC clone from chromosome 6p21 region was from our own library.

### Histone Extraction.

We prepared histones in accordance with established protocols1. We obtained histones from cell lines and tumor samples in parallel with their matching controls under identical conditions. For cell lines, we isolated nuclei with RSB buffer (10 mM Tris 10 (pH 7.5), 10 mM NaCl and 3 mM MgCl₂) containing I—Nonidet-P40 and protease inhibitors. We extracted nuclei with 0.25 M HCl and precipitated them with eight volumes of acetone. For tumors and their normal tissue counterparts, we homogenized samples with a Polytron homogenizer (Brinkman Instruments) in 0.25 M HCl and incubated them with gentle agitation for 4 h. We precipitated the resulting supernatant with eight volumes of acetone. We then fractionated individual histones by reverse-phase HPLC on a Jupiter C18 column. We made comparisons only between samples extracted with identical methodology.

### Western Blotting, Immunolocalization and Immunohistochemistry Assays.

For western blotting, we collected cells by centrifugation and washed cell pellets twice with phosphate-buffered saline buffer. For HDAC2 expression, nuclear extracts were fractionated on a 7.5% SDS-PAGE gel, transferred the fractions to a polyvinylidene difluoride membrane with 45-m pore size (Immobilon PSQ, Millipore), blocked the membrane in 5% milk PBS-T (phosphate-buffered saline with 0.1% Tween-20) and immunoprobed it with antibodies to HDAC2 (1:1000) and HDAC1 (1:1000; both from Abcam). The acetylated forms of histones 3 and 4 were analyzed as previously described2. We extracted the histones directly from the cell pellets with 0.250 M HCl. We fractionated 10 μg of the acid-extracted proteins on a 10% SDS-PAGE gel. Transferred membranes were immunoprobed with antibodies to acetylated H4 (1:2,000, Upstate) and acetylated H3 (1:50,000, Upstate). We used horseradish peroxidase-conjugated antibody to rabbit IgG (Amersham) at 1:3,000 dilution in PBS-T as the secondary antibody and antibody to H4 (1:3,000; Upstate) as a loading control. For immunolocalization experiments, we grew cells on coverslips and stained them with antibodies against HDAC2 and HDAC1 (Abcam) as previously described. We obtained images with a Leica DMRD photomicroscope coupled to a Leica DC200 camera, processed them using Adobe Photoshop software and analyzed them using public National Institutes of Health Image software. Immunohistochemical staining of HDAC2 was performed using a polyclonal antibody (Abcam, Cambridge, UK) at a 1:1500 dilution. After antigen retrieval with citrate buffer, immunodetection was performed by the DAKO EnVision Visualization Method (DAKO, Glostrup, Denmark), with diaminobenzidine chromogen as the substrate

### HPCE Quantification of Global Histone Acetylation.

We quantified global histone H4 acetylation as previously described3. We prepared individual histone fractions from cell nuclei and purified them by reverse-phase HPLC on a Jupiter C18 column (Phenomenex, Inc.) with an acetonitrile gradient (20-60%) in 0.3% trifluoroacetic acid, using a HPLC gradient system (Beckman-Coulter). We resolved non-, mono, di-, tri- and tetra-acetylated histone H4 derivatives by HPCE. We used an uncoated fused-silica capillary (Beckman-Coulter; 60.2 cm 75 μm, effective length 50 cm) in a capillary electrophoresis system (P/ACE MDQ, Beckman-Coulter) with 100 mM phosphate buffer (pH 2.0) 0.02% (w/v) HPM-cellulose as running buffer and operating voltages of 12 kV.

### HDAC2 Activity

For HDAC2 activity determinations, HDAC2 was immunoprecipitated as described elsewhere4 from nuclear extracts using the same antibody used for Western blotting and immunolocalization experiments. We then determined the HDAC2 activity by measuring released [3H] acetate in a scintillation counter after 1 hour incubations of HDAC2 immunoprecipitates at 37° C. with 3H-labeled histones4.

### Apoptosis and Cell Cycle Analysis

The percentage of apoptotic cells was determined by flow cytomery using Vybrant® Apoptosis Assay Kit #4-YO-PRO®-1/propidium iodide (Molecular Probes/Invitrogen). To analyze the cell cycle profiles, we stained with propidium iodide and the percentage of cells in G2/M was determined by flow cytometry.

### Cell Viability Assay

Cell viability was determined as previously described5. Aliquots of 1.5×104 cells were plated in 96-well microdilution plates. Following overnight cell adherence, experimental media containing the drugs or control media was added to appropriate wells. After 48 hours, the media was replaced by drug-free fresh media (100 μl/well) containing MTT (50 μg). After a 3 hour incubation at 37° C. in 5% CO₂ atmosphere, the MTT was removed and MTT-formazan crystals were dissolved in DMSO (100 μl/well). Absorbance at 570 nm was determined on an automatized microtiter plate reader. It was established that optical density was directly proportional to the cell number up to the density reached by the end of the assay.

### Analysis of HDAC2 CpG Island Promoter Methylation.

DNA samples were treated with sodium bisulfite and primers spanning the CpG island of HDAC2 promoter were used for bisulfite genomic sequencing. Primer sequences and PCR conditions are available upon request. Eighteen clones were analyzed.

### HDAC2 Transfection and Colony Formation Assay

The HDAC2 expression vector pcDNA3-HDAC2 was constructed by cloning the cDNA corresponding to the gene HDAC2 in pcDNA vector (Invitrogen). Transfection of RKO cells was performed by electroporating 107 cells in 0.8 ml PBS with 40 μg of the vector at 250 V and 975 μF. After electroporation, cells were washed with PBS and seeded with 106 cells/ml in fresh medium containing 20% FBS. C1ones expressing HDAC2 were selected in complete medium supplemented with 1 mg/mL G418. Stable clones were maintained in complete medium with 1 mg/mL G418. For colony formation experiments, stable G418-resistant colonies were fixed, stained with 2% methylene blue in 60% methanol, and the average number of colonies present in each well was determined.

HDAC2 siRNA

The HDAC2-specific siRNA was designed and synthesized by Qiagen. Two siRNA duplex were used against the HDAC2 gene that recognize the sequences 5′-CTG GGT TGT TTC AAT CTA ACA-3 and 5′-ACG GTC AAT AAG ACC AGA TAA-3′. Scramble siRNA was also purchased from Quigen and used as a control. Transfection was carried out using oligofectamine (Invitrogen) according to the manufacturer's specifications. At 12 h after transfections the cells were washed with fresh medium, and maintained in a 10% FBS-supplemented medium. The cells were harvested at specific times for total protein and histones extraction, and HDCA2 content was analyzed by western blotting and histone acetylation was quantified by HPCE.

### Mouse Xenograft Model.

Six-week-old female athymic nude mice nu/nu (Harlam Sprague-Dawley, Indianapolis, Ind.), housed under specific pathogen-free conditions (Institutional Animal Welfare Committee Agreement), were used for HCT116, RKO and Co115 tumor xenografts. For treatments, animals were randomly separated in three seven-specimens-groups, differentiated for PBS (Control), TSA (Trichostatin A) and BUT (Butyric acid) treatments. Both flanks of each animal were injected s.c. with 106 (HCT116) or 2×106 (RKO and CO115) cells in a total volume of 200 μL of PBS. For transfections assays both flanks of each animal were injected subcutaneously with 2×106 RKO (left) or RKO-HDAC2-transfected (right) cells in a total volume of 200 μL of PBS. Tumor development at the site of injection was evaluated daily. Animals were sacrificed at 21 days. The tumors were then excised and weighed.

### REFERENCES

1. Turner, B. M. & Fellows, G. Eur. J. Biochem. 179, 131-139 (1989).
2. Fraga, M. F. et al. Proc Natl Acad Sci USA. 102, 10604-10609 (2005).
3. Fraga, M. F. et al. Nat. Genet. 37, 391-400 (2005)
4. Espada, J. et al. J. Biol. Chem. 279, 37175-37184 (2004).
5. Ropero, S. et al. Breast Cancer Res. Treat. 86, 125-37 (2004).

### 4. Summary

Disruption of histone acetylation patterns is a common feature of cancer cells, although very little is known about its genetic basis. We have identified truncating mutations in one of the primary human histone deacetylases, HDAC2, in sporadic carcinomas with microsatellite instability, and in tumors arising in individuals with hereditary nonpolyposis colorectal cancer syndrome. The presence of the HDAC2 frameshift mutation causes a loss of HDAC2 protein expression and enzymatic activity, and renders these cells more resistant to the usual antiproliferative and proapoptotic effects of histone deacetylase inhibitors. Since such drugs may serve as cancer-therapeutic agents, our findings support the use of HDAC2 mutational status in future pharmacogenetic-oriented treatment of these patients.

In summary, we have demonstrated the presence of an inactivating mutation in the HDAC2 gene in human cancer cell lines and primary tumors with microsatellite instability that impairs these transformed cells' biochemical and cellular responses to trichostatin A, the archetypcal hydroxamic acid with histone deacetylase inhibitory activity. This finding supports the role of epigenetics, and especially of histone modifications, in tumorigenesis and it may have potential relevance for the pharmacogenetic selection of cancer patients treated with histone deacetylase inhibitors.

1. A method of diagnosing cancer comprising, in a sample obtained from a subject, determining the level or activity of HDAC2, wherein a reduced level or activity of HDAC2 is indicative of cancer.

2. The method of claim 1 wherein HDAC2 protein expression is determined.

3. The method of claim 2, wherein a loss of HDAC2 protein expression is indicative of cancer.

4. The method of claim 1 wherein analysis of gene expression of genes other than HDAC2 is utilised in order to determine the level or activity of HDAC2.

5. The method of claim 4 which comprises determining the levels of gene expression of a panel of genes, wherein the panel of genes comprises at least one gene selected from
(a) CARD6 (A_{—}23_P41854), TOP1MT (A_{—}23_P216241), SWAP70 (A_{—}23_P116533), KIAA1212 (A_{—}23_P17269), IRF5 (A_{—}23_P500271), CGI-90 (A_{—}23_P83492), UTS2 (A_{—}23_P63343), DJ462023.2 (A_{—}23_P46604), HHLA3 (A_{—}23_P200303), NFATC4 (A_{—}23_P140394), DKFZp686H1423 (A_{—}23_P392235), MGC17839 (A_{—}23_P329890), TSTA3 (A_{—}23_P94301), ZNF175 (A_{—}23_P332374), (A_{—}23_P119023), KIAA1212 (A_{—}23_P28664), LANCL1 (A_{—}23_P50887), FLJ38819 (A_{—}23_P385768), TAS2R9 (A_{—}23_P336506), ZNF22 (A_{—}23_P202458), SYT12 (A_{—}23_P424645), KIAA1826 (A_{—}23_P116166), ZHX1 (A_{—}23_P43150), BAIAP1 (A_{—}23_P96099), (A_{—}23_P150316), ZNF22 (A_{—}23_P384512), HMP19 (A_{—}23_P125375), M160 (A_{—}23_P61466), TFPI (A_{—}23_P17095), FJX1 (A_{—}23_P150693), (A_{—}23_P165598), EMP3 (A_{—}23_P119362), AP1S2 (A_{—}23_P217384), CCRL2 (A_{—}23_P69310), ZNF44 (A_{—}23_P119279), VAPB (A_{—}23_P91293), (A_{—}23_P28797), FLJ10458 (A_{—}23_P141315), FLJ10826 (A_{—}23_P3775), ZNF582 (A_{—}23_P395464), RBM9 (A_{—}23_P103091), C14orf168 (A_{—}23_P25913), KPNA4 (A_{—}23_P218835), FUNDC2 (A_{—}23_P171310), MGC29784 (A_{—}23_P255916), DEDD2 (A_{—}23_P141908), PHC1 (A_{—}23_P323094), CSTF1 (A_{—}23_P79882), SLIT2 (A_{—}23_P144348), EFHD2 (A_{—}23_P23443), MGC17986 (A_{—}23_P368777), ODF3L1 (A_{—}23_P357900), SGSH (A_{—}23_P254254), DEFB127 (A_{—}23_P385843), UCHL5 (A_{—}23_P148798), LTA4H (A_{—}23_P204593), MFN1 (A_{—}23_P253817), IKIP (A_{—}23_P53467), LTA4H (A_{—}23_P388670), EIF3S6 (A_{—}23_P43141), GSDM1 (A_{—}23_P152605), ZZANK1 (A_{—}23_P72725), KPNA5 (A_{—}23_P156443), CHRAC1 (A_{—}23_P123544), FBXW2 (A_{—}23_P254645), PTGFRN (A_{—}23_P114968), DUSP19 (A_{—}23_P90938), PLEKHB2 (A_{—}23_P28642), TNFAIP6 (A_{—}23_P165624), (A_{—}23_P57836), C20orf45 (A_{—}23_P210658), PRPS1 (A_{—}23_P95764), PLSCR3 (A_{—}23_P49597), LOC92689 (A_{—}23_P132914), TRIM38 (A_{—}23_P93236), BRD4 (A_{—}23_P425104), KIAA0649 (A_{—}23_P146497), FBXO28 (A_{—}23_P137578), PPP2R4 (A_{—}23_P60458), IRAK1 (A_{—}23_P73780), SSH2 (A_{—}23_P118712), EIF5A (A_{—}23_P100925), C9orf103 (A_{—}23_P123732), EEF1D (A_{—}23_P31838), PRPS2 (A_{—}23_P96641), ABCC9 (A_{—}23_P368691), WDFY2 (A_{—}23_P218108), MGC75360 (A_{—}23_P163373), LTK (A_{—}23_P14853), (A_{—}23_P348015), ZNF571 (A_{—}23_P108342), FLJ12517 (A_{—}23_P115523), (A_{—}23_P90070), FLJ31951 (A_{—}23_P167556), MGC29875 (A_{—}23_P46337), KIAA0103 (A_{—}23_P60000), LARS (A_{—}23_P218967), PEX19 (A_{—}23_P160188), GPR83 (A_{—}23_P202740), ATAD2 (A_{—}23_P216068), OR5AK2 (A_{—}23_P1863), OR6Q1 (A_{—}23_P161891), ITR (A_{—}23_P88021), MGC41816 (A_{—}23_P95027), NDUFB9 (A_{—}23_P157669), HSF1 (A_{—}23_P253841), FAM48A (A_{—}23_P140050), RPL10L (A_{—}23_P99754), KIAA1223 (A_{—}23_P81012), RRM2B (A_{—}23_P20223), LOC90806 (A_{—}23_P97697), LY6E (A_{—}23_P169077), FLII (A_{—}23_P4425), IL3 (A_{—}23_P122012), FLJ21168 (A_{—}23_P85993), BCL2L2 (A_{—}23_P140219), PIG8 (A_{—}23_P86822), SMARCA1 (A_{—}23_P44244), SCUBE1 (A_{—}23_P211619), CDYL2 (A_{—}23_P371871), GDPD1 (A_{—}23_P96060), SERAC1 (A_{—}23_P145419), C11orf23 (A_{—}23_P202637), FKBP10 (A_{—}23_P15727), WDR35 (A_{—}23_P108463), PPM1K (A_{—}23_P167138), NUDT12 (A_{—}23_P259090), DIAPH1 (A_{—}23_P213344), SIGIRR (A_{—}23_P84344), SCGN (A_{—}23_P251412), CTNNAL1 (A_{—}23_P157795), DIPA (A_{—}23_P150249), SGPP1 (A_{—}23_P347048), LOC196549 (A_{—}23_P311039), KREMEN2 (A_{—}23_P77612), FMNL2 (A_{—}23_P332744),
(b) ACP6 (A_{—}23_P160237), C3orf17 (A_{—}23_P336670), SLC10A5 (A_{—}23_P157428), (A_{—}23_P60758), ALG6 (A_{—}23_P35168), MAP4K3 (A_{—}23_P154130), ITLN2 (A_{—}23_P201419), PPIL4 (A_{—}23_P42507), RAE1 (A_{—}23_P346206), DDX20 (A_{—}23_P63153), CMKLR1 (A_{—}23_P105461), GRM2 (A_{—}23_P252184), DC12 (A_{—}23_P60947), (A_{—}23_P113983), PTPLB (A_{—}23_P155197), KIAA1639 (A_{—}23_P103734), MAD2L2 (A_{—}23_P23206), FLJ35382 (A_{—}23_P304410), OTOR (A_{—}23_P403385), MRPL47 (A_{—}23_P502427), (A_{—}23_P73451), CASC1 (A_{—}23_P95231), LOC128387 (A_{—}23_P23522), SBBI54 (A_{—}23_P67323), EDEM1 (A_{—}23_P40975), YARS (A_{—}23_P85570), CSNK2A2 (A_{—}23_P14915), MC1R (A_{—}23_P66095), MDS025 (A_{—}23_P162127), C14orf118 (A_{—}23_P54198), NIFIE14 (A_{—}23_P79043), (A_{—}23_P44514), OR5T1 (A_{—}23_P47444), TMF1 (A_{—}23_P143867), KNS2 (A_{—}23_P2873), (A_{—}23_P256903), LLGL1 (A_{—}23_P130266), (A_{—}23_P159277), C10orf117 (A_{—}23_P202496), TRAPPC3 (A_{—}23_P200535), SERPINA12 (A_{—}23_P88177), ARPC1A (A_{—}23_P82664), GTF2A1 (A_{—}23_P65609), ANKRD16 (A_{—}23_P150069), PTN (A_{—}23_P303085), FBXL20 (A_{—}23_P141146), AMPD2 (A_{—}23_P201591), CHRNG (A_{—}23_P17115), FLJ20507 (A_{—}23_P100155), FLJ35036 (A_{—}23_P212025), ARHGAP22 (A_{—}23_P75310), SENP2 (A_{—}23_P257988), LOC401137 (A_{—}23_P133011), BBS4 (A_{—}23_P99961), ETEA (A_{—}23_P156109), ITPKB (A_{—}23_P372255), TBC1D1 (A_{—}23_P73023), (A_{—}23_P147154), HSRG1 (A_{—}23_P206400), ZNF568 (A_{—}23_P67737), HCRTR1 (A_{—}23_P74178), LIPT1 (A_{—}23_P501805), HOXB3 (A_{—}23_P100872), GP9 (A_{—}23_P33256), XRN1 (A_{—}23_P132820), SF3A1 (A_{—}23_P104680), SCO1 (A_{—}23_P15466), GRIN2B (A_{—}23_P151264), Ufc1 (A_{—}23_P103905), PHF17 (A_{—}23_P167256), SF4 (A_{—}23_P119618), PLAA (A_{—}23_P135157), NDUFA11 (A_{—}23_P320185), RPL10L (A_{—}23_P341325), F8 (A_{—}23_P217643), SAGE1 (A_{—}23_P33343), ZFP28 (A_{—}23_P107673), MGC21654 (A_{—}23_P334218), (A_{—}23_P119652), FLJ11305 (A_{—}23_P25644), TRIP3 (A_{—}23_P383435), RHOF (A_{—}23_P203983), OR5AR1 (A_{—}23_P13273), C5orf3 (A_{—}23_P41912), EIF2B4 (A_{—}23_P154056), CPSF3 (A_{—}23_P28683), FLJ10774 (A_{—}23_P87329), GLUD1 (A_{—}23_P138665), KBTBD4 (A_{—}23_P202693), FLJ33167 (A_{—}23_P358470), RAB8B (A_{—}23_P37535), PAX9 (A_{—}23_P426944), SURF5 (A_{—}23_P501795), (A_{—}23_P46708), OR12D3 (A_{—}23_P259555), C9orf80 (A_{—}23_P397899), MAFF (A_{—}23_P103110), RND1 (A_{—}23_P53370), NPPA (A_{—}23_P74059), C9orf77 (A_{—}23_P83149), ATP6VOD1 (A_{—}23_P54636), IKBKB (A_{—}23_P216188), GUK1 (A_{—}23_P201097), COH1 (A_{—}23_P20298), NFATC3 (A_{—}23_P77440), C20orf128 (A_{—}23_P409888), CHES1 (A_{—}23_P106236), (A_{—}23_P86369), KRTHB1 (A_{—}23_P363769), LEP (A_{—}23_P31426), (A_{—}23_P136857), SERPINB6 (A_{—}23_P70348), FLJ11004 (A_{—}23_P118042), TDRD7 (A_{—}23_P123672), OR1C1 (A_{—}23_P86350), FBXO11 (A_{—}23_P90814), ST13 (A_{—}23_P68949), PRKAG1 (A_{—}23_P36513), PHKA1 (A_{—}23_P258531), FLJ32731 (A_{—}23_P112061), (A_{—}23_P119788), PSG3 (A_{—}23_P56354), PTPRT (A_{—}23_P135576), MOCOS (A_{—}23_P67042), SDSL (A_{—}23_P53439), FLJ21816 (A_{—}23_P129569), KATNA1 (A_{—}23_P113803), BRDT (A_{—}23_P147976), FNTA (A_{—}23_P24926), RAB3B (A_{—}23_P62672), CTAG1B (A_{—}23_P148541), GLE1L (A_{—}23_P258367), ADCY7 (A_{—}23_P106949), ABI1 (A_{—}23_P126992), TCEA1 (A_{—}23_P132444), BTBD5 (A_{—}23_P205659), (A_{—}23_P149398), (A_{—}23_P158524), CDCA4 (A_{—}23_P205449), NUTF2 (A_{—}23_P118038), OR1B1 (A_{—}23_P146611), PDE6B (A_{—}23_P10743), HSC20 (A_{—}23_P40588), RNF8 (A_{—}23_P70384), (A_{—}23_P254382), ALDH9A1 (A_{—}23_P11884), BCL6B (A_{—}23_P130130), PPP1R12C (A_{—}23_P50575), GCM1 (A_{—}23_P133728), ARHGAP17 (A_{—}23_P3716), C9orf10 (A_{—}23_P32294), DR1 (A_{—}23_P63205), SPR (A_{—}23_P68208), RAB9P40 (A_{—}23_P123814), TTC19 (A_{—}23_P164421), PRKAA1 (A_{—}23_P110725), LOC127253 (A_{—}23_P103180), FANCE (A_{—}23_P42335), BMP15 (A_{—}23_P11107), HSPG2 (A_{—}23_P23191), POLD1 (A_{—}23_P50456), SLC3A1 (A_{—}23_P165325), PRUNE (A_{—}23_P169925), SF3B3 (A_{—}23_P135914), OXT (A_{—}23_P57133), EEF1G (A_{—}23_P13344), MGC2494 (A_{—}23_P54728), SUV39H2 (A_{—}23_P202392), WDR3 (A_{—}23_P23318), PLP2 (A_{—}23_P251089), GRM5 (A_{—}23_P24361), FLJ14007 (A_{—}23_P146050), GFER (A_{—}23_P206484), LASS4 (A_{—}23_P153867), C9orf16 (A_{—}23_P158048), ITIH1 (A_{—}23_P18223), CYP17A1 (A_{—}23_P75111), ACTN3 (A_{—}23_P138881), FLJ39378 (A_{—}23_P303293), GALNACT-2 (A_{—}23_P149887), COBRA1 (A_{—}23_P148150), COPS8 (A_{—}23_P144257), LOC-155060 (A_{—}23_P254441), DGKI (A_{—}23_P111432), SLC22A6 (A_{—}23_P98616), SH3BP2 (A_{—}23_P110116), RTTN (A_{—}23_P337896), LOC51255 (A_{—}23_P108708), FLNA (A_{—}23_P96559), HSD17B12 (A_{—}23_P47377), EBAG9 (A_{—}23_P255226), PPP1R16A (A_{—}23_P157715), FIBP (A_{—}23_P13018), SH3GLB2 (A_{—}23_P216995), PHF20L1 (A_{—}23_P134786), C10orf9 (A_{—}23_P434-419), CEP1 (A_{—}23_P32183), NDUFV2 (A_{—}23_P130418), LOC147804 (A_{—}23_P55854), TOMM34 (A_{—}23_P57033), MSL3L1 (A_{—}23_P217776), TOPORS (A_{—}23_P32217), S100A13 (A_{—}23_P372874), GBA (A_{—}23_P201030), C21orf55 (A_{—}23_P68700), IFNGR2 (A_{—}23_P29034), SNAPC1 (A_{—}23_P37251), OR56B1 (A_{—}23_P139244), APOA1BP (A_{—}23_P126446), SUHW3 (A_{—}23_P314642), C2orf33 (A_{—}23_P405148), SSSCA1 (A_{—}23_P98261), CKLFSF7 (A_{—}23_P256413), NBEAL1 (A_{—}23_P217068), (A_{—}23_P49725), ZNF45 (A_{—}23_P426-472), LOC400986 (A_{—}23_P131322), CHURC1 (A_{—}23_P37237), GSS (A_{—}23_P210920), CD96 (A_{—}23_P32770), C21orf61 (A_{—}23_P57293), DERL1 (A_{—}23_P216043), ACAD8 (A_{—}23_P47426), MGC14595 (A_{—}23_P157679), ALDOA (A_{—}23_P88961), SLC39A4 (A_{—}23_P20502), JRK (A_{—}23_P8930), AAMP (A_{—}23_P56529), FERD3L (A_{—}23_P422849), PRKCSH (A_{—}23_P208615), TCTE1L (A_{—}23_P73577), EPHB1 (A_{—}23_P166994), FAM49B (A_{—}23_P43255), HSU79274 (A_{—}23_P65000), PRDX6 (A_{—}23_P983), LOC400986 (A_{—}23_P210134), ARL10C (A_{—}23_P212229), OSGEP (A_{—}23_P54078), LOR (A_{—}23_P22297), BCKDK (A_{—}23_P365149),
(c) PTPN1 (A_{—}23_P338890), RBKS (A_{—}23_P9523), DEFB119 (A_{—}23_P413089), PARP11 (A_{—}23_P343837), SLC12A1 (A_{—}23_P99879), CEP2 (A_{—}23_P102832), CREB5 (A_{—}23_P 157117), GPX5 (A_{—}23_P214544), STATH (A_{—}23_P252253), MGC2474 (A_{—}23_P3819), AP2A1 (A_{—}23_P164889), KLF15 (A_{—}23_P40809), ZMYND19 (A_{—}23_P305173), EIF3S4 (A_{—}23_P38887), COL8A1 (A_{—}23_P80436), IFIT5 (A_{—}23_P63668), NCB50R (A_{—}23_P30995), DMRT2 (A_{—}23_P365694), (A_{—}23_P2180), TRY1 (A_{—}23_P82558), DVL1 (A_{—}23_P347432), CNDP1 (A_{—}23_P9869), ADA (A_{—}23_P210482), LENG1 (A_{—}23_P208520), (A_{—}23_P25534), STX4A (A_{—}23_P256375), FLJ1171 (A_{—}23_P106753), CAPG (A_{—}23_P165636), BNC2 (A_{—}23_P43684), LOC387758 (A_{—}23_P138885), NCE2 (A_{—}23_P39561), MDGA1 (A_{—}23_P310460), OTUB1 (A_{—}23_P24375), FLJ38348 (A_{—}23_P339633), ZBTB9 (A_{—}23_P8116), CAP2 (A_{—}23_P156417), OR1Si (A_{—}23_P52957), CLCA3 (A_{—}23_P34382), (A_{—}23_P94020), IRAK2 (A_{—}23_P80635), CITED4 (A_{—}23_P74155), FLJ21369 (A_{—}23_P50597), POLE4 (A_{—}23_P154234), SH3GLB1 (A_{—}23_P22957), PROL5 (A_{—}23_P41365), DKFZp547E052 (A_{—}23_P102048), SLC4A7 (A_{—}23_P132468), LETM2 (A_{—}23_P348264), RABL3 (A_{—}23_P369393), OR51TI (A_{—}23_P24856), DNCI2 (A_{—}23_P154108), FLJ20534 (A_{—}23_P60510), FLJ10979 (A_{—}23_P130285), (A_{—}23_P135946), WASF3 (A_{—}23_P151351), PSG6 (A_{—}23_P108170), CDY1 (A_{—}23_P62471), TNFRSF12A (A_{—}23_P49338), C10orf97 (A_{—}23_P23983), CPSF5 (A_{—}23_P129614), FLJ38944 (A_{—}23_P368259), Bles03 (A_{—}23_P150238), ADRB3 (A_{—}23_P168993), DNAJC8 (A_{—}23_P37137), SNX17 (A_{—}23_P28233), MSR1 (A_{—}23_P216176), ARL8 (A_{—}23_P378588), (A_{—}23_P71709), RRN3 (A_{—}23_P206877), SLN (A_{—}23_P 150343), C1QTNF4 (A_{—}23_P52597), SCARF1 (A_{—}23_P15414), NUDT5 (A_{—}23_P1196), ELOVL6 (A_{—}23_P7361), RAD54B (A_{—}23_P94141), C3orf20 (A_{—}23_P361333), OLFML1 (A_{—}23_P147664), ST7L (A_{—}23_P310582), (A_{—}23_P169587), POLE4 (A_{—}23_P383385), PFDN2 (A_{—}23_P51906), FBXL6 (A_{—}23_P43296), MYC (A_{—}23_P215956), FMO1 (A_{—}23_P12386), KIAA1446 (A_{—}23_P253614), HSPA4L (A_{—}23_P363936), DCP2 (A_{—}23_P256868), THRAP6 (A_{—}23_P31866), (A_{—}23_P 158699), DNAJB4 (A_{—}23_P51339), LECT2 (A_{—}23_P110777), ZNF45 (A_{—}23_P101721), KCNQ2 (A_{—}23_P210400), CALML5 (A_{—}23_P124095), RAD23A (A_{—}23_P55889), FLJ32784 (A_{—}23_P406-478), FLJ13220 (A_{—}23_P155959), FANCF (A_{—}23_P12896), (A_{—}23_P124703), GCDH (A_{—}23_P90089), CYP27A1 (A_{—}23_P131308), LIPG (A_{—}23_P78405), APBA2BP (A_{—}23_P68628), FBXL4 (A_{—}23_P214739), PSMD5 (A_{—}23_P43434), RGS11 (A_{—}23_P118124), LTB4DH (A_{—}23_P 157809), FLJ20557 (A_{—}23_P55688), JARID1B (A_{—}23_P409866), (A_{—}23_P133049), QDPR (A_{—}23_P167212), NCOA4 (A_{—}23_P86424), ZNF25 (A_{—}23_P381577), FLJ21934 (A_{—}23_P213279), RP1 (A_{—}23_P71368), FLJ23790 (A_{—}23_P411215), TRIM3 (A_{—}23_P150619), C9orf27 (A_{—}23_P123702), C20orf43 (A_{—}23_P143258), PRPS1L1 (A_{—}23_P418516), MGC2655 (A_{—}23_P37949), OR1N1 (A_{—}23_P169376), EIF2C2 (A_{—}23_P112159), ALS2CR2 (A_{—}23_P90682), RBPMS (A_{—}23_P71316), RBMS2 (A_{—}23_P36432), (A_{—}23_P65843), LOC51236 (A_{—}23_P61268), (A_{—}23_P20372), EIF3S3 (A_{—}23_P9061), OGFRL1 (A_{—}23_P7791), FLJ31547 (A_{—}23_P118234), CPXM2 (A_{—}23_P138524), ZNF382 (A_{—}23_P16354), IRX2 (A_{—}23_P156025), DKFZP56400463 (A_{—}23_P215875), (A_{—}23_P340640), (A_{—}23_P80788), FLJ10178 (A_{—}23_P96369), (A_{—}23_P32966), SCHIP1 (A_{—}23_P257031), SLC6A6 (A_{—}23_P69206), C2orf3 (A_{—}23_P120064), OR1K1 (A_{—}23_P251528), CD207 (A_{—}23_P39790), THSD6 (A_{—}23_P5246), HIST1H2AI (A_{—}23_P168008), TAS2R16 (A_{—}23_P59825), SOAT1 (A_{—}23_P63319), NET-5 (A_{—}23_P151127), HPCAL1 (A_{—}23_P5831), C4BPA (A_{—}23_P97541), TBCD (A_{—}23_P100602), CTAG3 (A_{—}23_P361509), DEFB125 (A_{—}23_P320846), FLJ23588 (A_{—}23_P68978), CXorf1 (A_{—}23_P430747), DAPK1 (A_{—}23_P252163), SIAT2 (A_{—}23_P131455), (A_{—}23_P84475), DBCCR1L (A_{—}23_P103812), TNFRSF10D (A_{—}23_P95417), CORO1C (A_{—}23_P53456), OR11 H1 (A_{—}23_P128866), FLJ32731 (A_{—}23_P147950), ERCC1 (A_{—}23_P107701), ZNF550 (A_{—}23_P354827), IQCG (A_{—}23_P124381), DYRK3 (A_{—}23_P12282),
(d) ASB11 (A_{—}23_P114259), (A_{—}23_P81273), OR52A1 (A_{—}23_P125286), LOXHD1 (A_{—}23_P107531), SPTBN2 (A_{—}23_P98282), CNNM2 (A_{—}23_P24044), ZNF71 (A_{—}23_P119068), PITPNB (A_{—}23_P17786), ANAPC2 (A_{—}23_P253062), SLC32AI (A_{—}23_P154561), C20orf178 (A_{—}23_P28964), TSP-NY (A_{—}23_P2258), FLJ12525 (A_{—}23_P256021), ABO (A_{—}23_P112645), ADCK4 (A_{—}23_P208409), FLJ22405 (A_{—}23_P69229), (A_{—}23_P169341), PTHR2 (A_{—}23_P28181), LOC51058 (A_{—}23_P97221), (A_{—}23_P72359), RUTBC3 (A_{—}23_P166491), EXOSC7 (A_{—}23_P58102), FOXD3 (A_{—}23_P46560), ERF (A_{—}23_P4678), MCOLN1 (A_{—}23_P27571), FLJ38377 (A_{—}23_P313010), CD40 (A_{—}23_P57036), RPP38 (A_{—}23_P150080), NUP160 (A_{—}23_P43726), CLC (A_{—}23_P101684), RPP21 (A_{—}23_P214594), HSPA6 (A_{—}23_P114903), MAP3K11 (A_{—}23_P98273), TBX15 (A_{—}23_P62981), STAT5B (A_{—}23_P100788), (A_{—}23_P23673), EAP30 (A23—P130064), (A_{—}23_P133087), RYK (A_{—}23_P388081), RCP9 (A_{—}23_P361542), RYK (A_{—}23_P22001), SCN4B (A_{—}23_P303833), DUSP10 (A_{—}23_P51861), ACTL7B (A_{—}23_P9186), OLIG2 (A_{—}23_P211079), CT120 (A_{—}23_P50000), (A_{—}23_P33326), FARSLA (A_{—}23_P78682), (A_{—}23_P69129), CAPN14 (A_{—}23_P56450), PLA2G2E (A_{—}23_P114857), C21orf86 (A_{—}23_P417294), TTLL2 (A_{—}23_P7901), CYP2W1 (A_{—}23_P31437), CCR8 (A_{—}23_P211699), ZNFN1A5 (A_{—}23_P35667), FLJ35155 (A_{—}23_P345708), PPP2CB (A_{—}23_P308097), KIAA0100 (A_{—}23_P207614), (A_{—}23_P114895), DKFZP586A0522 (A_{—}23_P128432), MAK (A_{—}23_P214839), PS1D (A_{—}23_P96976), EMR4 (A_{—}23_P44380), SYTL4 (A_{—}23_P11136), DHODH (A_{—}23_P15202), LOC116441 (A_{—}23_P91885), DNAJC4 (A_{—}23_P202769), XPR1 (A_{—}23_P63534), POLR2C (A_{—}23_P3527), SFXN4 (A_{—}23_P387722), CRK7 (A_{—}23_P350093), KCNJ15 (A_{—}23_P17655), FBXO11 (A_{—}23_P79416), SLC22A9 (A_{—}23_P150590), CACNG5 (A_{—}23_P66497), IL1RAPL2 (A_{—}23_P255038), MRAS (A_{—}23_P377197), MGC16733 (A_{—}23_P203737), FLJ38716 (A_{—}23_P343366), TRA@ (A_{—}23_P106061), MGC 11134 (A_{—}23_P98244), SEC10L1 (A_{—}23_P14464), SEZ6 (A_{—}23_P389569), GRPEL1 (A_{—}23_P92476), TOSO (A_{—}23_P138125), FBXL12 (A_{—}23_P78554), ACTA1 (A_{—}23_P1102), FGFR4 (A_{—}23_P92754), TCBA1 (A_{—}23_P170209), FLJ10246 (A_{—}23_P144704), DEAF1 (A_{—}23_P158533), (A_{—}23_P29296), SLC9A8 (A_{—}23_P252936), MS4A6E (A_{—}23_P416234), SELK (A_{—}23_P364517), SCGB1D4 (A_{—}23_P98598), TM9SF4 (A_{—}23_P210872), DHX32 (A_{—}23_P47004), PRPSAP2 (A_{—}23_P49646), HELB (A_{—}23_P2294), LTC4S (A_{—}23_P92802), PGRMC2 (A_{—}23_P155868), BLZF1 (A_{—}23_P23266), NDFIP1 (A_{—}23_P81247), SUPT6H (A_{—}23_P141479), CECR6 (A_{—}23_P259344), CNGA4 (A_{—}23_P2006), C14orf119 (A_{—}23_P117447), (A_{—}23_P17984), PAX7 (A_{—}23_P126225), DR1 (A_{—}23_P391725), XAB1 (A_{—}23_P17144), BMP8A (A_{—}23_P126508), C1orf26 (A_{—}23_P96931), TRPM1 (A_{—}23_P129225), ALG3 (A_{—}23_P7005), OR5D14 (A_{—}23_P36057), C2orf29 (A_{—}23_P108761), MGC26717 (A_{—}23_P44177), SHBG (A_{—}23_P207088), RAMP3 (A_{—}23_P111737), OR1J2 (A_{—}23_P157996), CLSPN (A_{—}23_P126207), MOV10 (A_{—}23_P161125), (A_{—}23_P155997), MS4A8B (A_{—}23_P139147), BCAR1 (A_{—}23_P77724), SSX4 (A_{—}23_P254202), FLJ14129 (A_{—}23_P60101), HDAC8 (A_{—}23_P84922), ORC3L (A_{—}23_P42045), GFRA2 (A_{—}23_P84084), AFG3L2 (A_{—}23_P135454), GPR45 (A_{—}23_P131534), PBOV1 (A_{—}23_P255942), PRKCE (A_{—}23_P250564), PPP2CZ (A_{—}23_P201939), DAP3 (A_{—}23_P63067), CTNS (A_{—}23_P427075), SMARCA5 (A_{—}23_P256716), FRS2 (A_{—}23_P430785), CROCC (A_{—}23_P104086), PLK4 (A_{—}23_P155968), CSIG (A_{—}23_P54597), SLC35F2 (A_{—}23_P339098), NT5C2L1 (A_{—}23_P382043), IL3 (A_{—}23_P348828), ZNF434 (A_{—}23_P218283), KIR2DS5 (A_{—}23_P381532), (A_{—}23_P72169), GML (A_{—}23_P59976), RBM7 (A_{—}23_P138975), SIGLEC7 (A_{—}23_P50182), SRPK2 (A_{—}23_P215594), B4GALT3 (A_{—}23_P103912), XRN2 (A_{—}23_P166135), PARP4 (A_{—}23_P117172), PTBP1 (A_{—}23_P208981), (A_{—}23_P33148), GPRC6A (A_{—}23_P81683), KIAA0063 (A_{—}23_P6479), WDR12 (A_{—}23_P28625), HIRIP5 (A_{—}23_P147296), ARHGAP27 (A_{—}23_P141336), DONSON (A_{—}23_P500390), PRDM9 (A_{—}23_P7612), ATP7B (A_{—}23_P205228), IL15RA (A_{—}23_P138680), (A_{—}23_P166910), FLJ35801 (A_{—}23_P433719), MAPK14 (A_{—}23_P214696), GALE (A_{—}23_P160148), OVCH1 (A_{—}23_P435974), (A_{—}23_P170925), LRCH2 (A_{—}23_P73809), HAL (A_{—}23_P61643), (A_{—}23_P164520), CNOT4 (A_{—}23_P156993), SIRT2 (A_{—}23_P142455), KIAA1984 (A_{—}23_P302410), ZNF497 (A_{—}23_P27414), (A_{—}23_P84555), CCNB3 (A_{—}23_P171107), SLC8A1 (A_{—}23_P119957), TUSC4 (A_{—}23_P18267), ZP1 (A_{—}23_P1912), AKR7A3 (A_{—}23_P103968), ZCWCC3 (A_{—}23_P325501), RAB7L1 (A_{—}23_P126939), GP6 (A_{—}23_P27784), ZNF394 (A_{—}23_P157416), RPS9 (A_{—}23_P208535), GTF2H1 (A_{—}23_P36183), GPR135 (A_{—}23_P205575), SLC6A16 (A_{—}23_P130735), HKE2 (A_{—}23_P122563), TUBG1 (A_{—}23_P152768), ARD1 (A_{—}23_P148546), STAMBP (A_{—}23_P28555), (A_{—}23_P154166), RPL10 (A_{—}23_P217666), ROPN1L (A_{—}23_P121885), PAK3 (A_{—}23_P346813), ZNF235 (A_{—}23_P208325), RBBP9 (A_{—}23_P57181), EXOSC9 (A_{—}23_P81121), STK25 (A_{—}23_P252653), (A_{—}23_P94711), ZNF485 (A_{—}23_P115861), WBSCR18 (A_{—}23_P157168), ENDOG (A_{—}23_P83266), RBL1 (A_{—}23_P28733), C21orf70 (A_{—}23_P61202), (A_{—}23_P210784), (A_{—}23_P98669), KCNJ6 (A_{—}23_P29058), USP32 (A_{—}23_P107154), (A_{—}23_P31829), C21orf129 (A_{—}23_P413696), SLC31A1 (A_{—}23_P253409), ZNF192 (A_{—}23_P214529), OR3A2 (A_{—}23_P55504), CLNS1A (A_{—}23_P127995), ZFP106 (A_{—}23_P77310), USP47 (A_{—}23_P148204), ID12 (A_{—}23_P52543), PTPN3 (A_{—}23_P403898), AUP1 (A_{—}23_P253421), THG-1 (A_{—}23_P250865), MKLN1 (A_{—}23_P93613), (A_{—}23_P29866), LOC90353 (A_{—}23_P303320), FLJ20729 (A_{—}23_P201396), (A_{—}23_P10753), PGD (A_{—}23_P126623), TNRC15 (A_{—}23_P142950), SMYD5 (A_{—}23_P91015), EHBP1L1 (A_{—}23_P148114), PAF53 (A_{—}23_P9455), TCEB2 (A_{—}23_P312181), NSUN6 (A_{—}23_P61580), WNT7A (A_{—}23_P258410), P29 (A_{—}23_P45756), BTBD14A (A_{—}23_P113825), GALNT2 (A_{—}23_P806), CD2AP (A_{—}23_P156484), FLJ39378 (A_{—}23_P169934), SUV39H1 (A_{—}23_P422193), IL24 (A_{—}23_P51951), HOXC6 (A_{—}23_P150974), SLC17A2 (A_{—}23_P59048), GLUD2 (A_{—}23_P45361), CDC14A (A_{—}23_P201921), UBE2A (A_{—}23_P148446), RANGNRF (A_{—}23_P55136), RANBP3 (A_{—}23_P208973), TUFM (A_{—}23_P251209), NLGN3 (A_{—}23_P62303), PACSIN2 (A_{—}23_P80377), DGCR8 (A_{—}23_P211355), EMD (A_{—}23_P85171), HERC4 (A_{—}23_P202408), EEF2 (A_{—}23_P5027), KIAA1754 (A_{—}23_P340333), (A_{—}23_P112825), HEBP1 (A_{—}23_P117082), SNRPB (A_{—}23_P 154675), BRUNOL5 (A_{—}23_P60766), C6orf89 (A_{—}23_P396842), JMJD1C (A_{—}23_P86434), MGC42638 (A_{—}23_P367071), HMG20B (A_{—}23_P119543), C9orf98 (A_{—}23_P83200), (A_{—}23_P89012), SCRT1 (A_{—}23_P147782), PLXNA4 (A_{—}23_P111448), MYH13 (A_{—}23_P89334), GCET2 (A_{—}23_P253245), RNF20 (A_{—}23_P216850), ZDHHC13 (A_{—}23_P13065), PPP4C (A_{—}23_P100355), OR1L8 (A_{—}23_P157991), UBQLN3 (A_{—}23_P98830), SH3BGR (A_{—}23_P91520), DPAGT1 (A_{—}23_P1775), FLJ35740 (A_{—}23_P310552), LOC51333 (A_{—}23_P416836), COH1 (A_{—}23_P359173), ATOH1 (A_{—}23_P332246), ZG16 (A_{—}23_P49145), E2F7 (A_{—}23_P322426), STAM (A_{—}23_P1343), ATP2B2 (A_{—}23_P18153), HLA-DOA (A_{—}23_P30923), HT007 (A_{—}23_P2124), 1HPK1 (A_{—}23_P250537), SCIRP10 (A_{—}23_P372434), MYOZ1 (A_{—}23_P1320), SEZ6L (A_{—}23_P80242), LCMT1 (A_{—}23_P124213), C21orf61 (A_{—}23_P304554), ALPL (A_{—}23_P97043), OAZIN (A_{—}23_P157435), SLC34A2 (A_{—}23_P121646), ZNF505 (A_{—}23_P135826), AQP9 (A_{—}23_P106362), HTR3B (A_{—}23_P87007), RABGEF1 (A_{—}23_P250824), GPRC5D (A_{—}23_P105691), (A_{—}23_P305581), OR51A4 (A_{—}23_P218015), TRPC4 (A_{—}23_P105873), (A_{—}23_P113263), OR51B6 (A_{—}23_P47774), SLC36A1 (A_{—}23_P167640), AGTRAP (A_{—}23_P147215), C20orf30 (A_{—}23_P17482), (A_{—}23_P146679), DSC1 (A_{—}23_P38696), POLR3A (A_{—}23_P149826), FIS (A_{—}23_P408323), RUVBL2 (A_{—}23_P39110), ELAVL1 (A_{—}23_P388681), KCNQ3 (A_{—}23_P123393), GPR119 (A_{—}23_P21425), SEC10L1 (A_{—}23_P413826), TMEM7 (A_{—}23_P57910), C20orf116 (A_{—}23_P403968), GGN (A_{—}23_P333552), OR8S1 (A_{—}23_P53378), NEK3 (A_{—}23_P76718), FGF1 (A_{—}23_P213334), WDR1 (A_{—}23_P213001), MAFG (A_{—}23_P207759), PLB1 (A_{—}23_P56356), BAK1 (A_{—}23_P145357), KCNA10 (A_{—}23_P126528), HPRP8BP (A_{—}23_P45913), C6orf29 (A_{—}23_P93352), DBT (A_{—}23_P74022), NCOA61P (A_{—}23_P60899), MTNR1A (A_{—}23_P80987), PSMB2 (A_{—}23_P170058), ASAHL (A_{—}23_P155666), PSMD11 (A_{—}23_P207600), ANP32C (A_{—}23_P92524), RBM16 (A_{—}23_P111303), LOC126295 (A_{—}23_P39263), IMP4 (A_{—}23_P302094), ADAM2 (A_{—}23_P73441), EBNA1BP2 (A_{—}23_P103631), MYOZ3 (A_{—}23_P58686), LOC92912 (A_{—}23_P77274), UMP-CMPK (A_{—}23_P115366), KIAA0748 (A_{—}23_P105423), CRYGB (A_{—}23_P414842), GDAP1L1 (A_{—}23_P17356), MUS81 (A_{—}23_P161634), FLJ30656 (A_{—}23_P307430), KCNN4 (A_{—}23_P67529), SLC16A1 (A_{—}23_P126825), DNAJC5B (A_{—}23_P8959), CGI-94 (A_{—}23_P11774), DPT (A_{—}23_P200741), RHPN1 (A_{—}23_P87438), CRIM1 (A_{—}23_P51105), SPP2 (A_{—}23_P90829), (A_{—}23_P73264), STAT6 (A_{—}23_P47879), IL22 (A_{—}23_P117115), LOC142937 (A_{—}23_P402164), SMYD3 (A_{—}23_P51414), FLJ10808 (A_{—}23_P218905), FLJ20184 (A_{—}23_P252276), WNT7B (A_{—}23_P147544), NARG1 (A_{—}23_P212825), MGC10067 (A_{—}23_P400344), SEC22L2 (A_{—}23_P259874), (A_{—}23_P81092), LOC285590 (A_{—}23_P122104), PROK1 (A_{—}23_P51745), TSSC4 (A_{—}23_P2023), EYA1 (A_{—}23_P502363), ARL2L1 (A_{—}23_P10081), TXNDC (A_{—}23_P309711), TNFSF8 (A_{—}23_P169257), BRAP (A_{—}23_P53614), SERPINH1 (A_{—}23_P75998), RASAL2 (A_{—}23_P86124), PRKDC (A_{—}23_P169612), PDE7B (A_{—}23_P59338), MRPL4 (A_{—}23_P164702), HTR2B (A_{—}23_P16953), NDUFB7 (A_{—}23_P50872), TUFM (A_{—}23_P9582), NIT2 (A_{—}23_P110099), PLK3 (A_{—}23_P51646), FLJ11267 (A_{—}23_P 168965), LOC55971 (A_{—}23_P 168470), CPSF6 (A_{—}23_P204039), PAQR4 (A_{—}23_P66213), MT (A_{—}23_P132358), EIF3S5 (A_{—}23_P142774), CRIPT (A_{—}23_P17219), APOBEC3B (A_{—}23_P369966), CBX6 (A_{—}23_P40677), ANKRD13 (A_{—}23_P36738), KIAA1900 (A_{—}23_P252521), AURKB (A_{—}23_P130182), (A_{—}23_P36439), RAB13 (A_{—}23_P46365), DUSP24 (A_{—}23_P111635), FLJ40137 (A_{—}23_P55196), SARS (A_{—}23_P126790), FLJ32830 (A_{—}23_P430788), N-PAC (A_{—}23_P396785), FLJ35700 (A_{—}23_P342165), MGC24039 (A_{—}23_P379746), TLK1 (A_{—}23_P39684), MI-ER1 (A_{—}23_P305723), PAQR3 (A_{—}23_P167276), ACTR2 (A_{—}23_P377376), PSFL (A_{—}23_P205997), CLIC4 (A_{—}23_P259189), CCRN4L (A_{—}23_P144338), IMPA1 (A_{—}23_P168818), FLJ20989 (A_{—}23_P33113), LOC124446 (A_{—}23_P77562), RPL28 (A_{—}23_P208356), BANF1 (A_{—}23_P47210), RAB37 (A_{—}23_P163972), (A_{—}23_P22639), TCBA1 (A_{—}23_P316501), HADHA (A_{—}23_P159510), WDR5 (A_{—}23_P32558), FLJ12442 (A_{—}23_P44836), DKFZP547N043 (A_{—}23_P433791), MPV17 (A_{—}23_P143084), KIAA0256 (A_{—}23_P37416), (A_{—}23_P158837), ADAMTS5 (A_{—}23_P40415), PTD008 (A_{—}23_P218486), UPK2 (A_{—}23_P64243), TERF1 (A_{—}23_P216149), STIP1 (A_{—}23_P124470), LASS5 (A_{—}23_P76515), OR5T3 (A_{—}23_P75710), TMED5 (A_{—}23_P86002), CCR4 (A_{—}23_P72989), (A_{—}23_P131887), (A_{—}23_P158605), AATF (A_{—}23_P89460), PME-1 (A_{—}23_P64567), (A_{—}23_P57626), RBM8A (A_{—}23_P104116), APG4B (A_{—}23_P9903), CHCHD6 (A_{—}23_P91870), ATF6 (A_{—}23_P62907), OR52N5 (A_{—}23_P64474), (A_{—}23_P154442), LPP (A_{—}23_P251118), GPR62 (A_{—}23_P434289), PFKM (A_{—}23_P170848), MGC13379 (A_{—}23_P203389), ATRX (A_{—}23_P136874), KIAA1324 (A_{—}23_P115392), TP53BP2 (A_{—}23_P12523), ARL8 (A_{—}23_P161407), GRB2 (A_{—}23_P418537), PDCD2 (A_{—}23_P145146), (A_{—}23_P74368), ZNF224 (A_{—}23_P22286), (A_{—}23_P206032), DKFZp566CO424 (A_{—}23_P 115106), (A_{—}23_P252796), (A_{—}23_P81780), GFRA3 (A_{—}23_P41992), KIF13B (A_{—}23_P95441), MGC40214 (A_{—}23_P330486), ITLN1 (A_{—}23_P84388), FXYD2 (A_{—}23_P161769), MGC5391 (A_{—}23_P5566), PABPC3 (A_{—}23_P48314), TRIM25 (A_{—}23_P15326), HIAT1 (A_{—}23_P45851), IFNA17 (A_{—}23_P 169307), PMS1 (A_{—}23_P40063), MGC4645 (A_{—}23_P 159071), LOC65121 (A_{—}23_P86072), DRD5 (A_{—}23_P374129), PTPRC (A_{—}23_P12392), ACTN1 (A_{—}23_P105957), LOC221143 (A_{—}23_P151368), ANKRD12 (A_{—}23_P130293), (A_{—}23_P43630), MAN2B1 (A_{—}23_P27613), E2F5 (A_{—}23_P31713), ROBO4 (A_{—}23_P344421), ZNF70 (A_{—}23_P154981), HIST1H1T (A_{—}23_P133842), B3GALT6 (A_{—}23_P35021), SCRN3 (A_{—}23_P17015), UCK2 (A_{—}23_P487), TAS2R43 (A_{—}23_P371254), SHMT2 (A_{—}23_P158239), OR4D1 (A_{—}23_P66392), PPP4R2 (A_{—}23_P41159), SLC25A1 (A_{—}23_P120773), TAF2 (A_{—}23_P43248), LY6G5C (A_{—}23_P145323), NPY5R (A_{—}23_P251836), WDR18 (A_{—}23_P5115), NOD3 (A_{—}23_P329399), TRIP12 (A_{—}23_P147984), CDC37 (A_{—}23_P130527), GLP2R (A_{—}23_P141309), HTR6 (A_{—}23_P74766), APG7L (A_{—}23_P143992), PQBP1 (A_{—}23_P62136), CAMLG (A_{—}23_P213728), WDSAM1 (A_{—}23_P108657), SLC35B3 (A_{—}23_P145463), FLJ20457 (A_{—}23_P216564), DOC2A (A_{—}23_P340953), KIAA1972 (A_{—}23_P3784), (A_{—}23_P52517), FLJ00012 (A_{—}23_P36305), LOC51252 (A_{—}23_P258992), (A_{—}23_P70077), TRAR1 (A_{—}23_P93531), IL17D (A_{—}23_P345692), NOG (A_{—}23_P341938), FLJ36749 (A_{—}23_P387624), OR2B2 (A_{—}23_P111088), PLD3 (A_{—}23_P27515), ADAMTSL3 (A_{—}23_P43940), TPCN1 (A_{—}23_P423208), TMEM30A (A_{—}23_P70290), MC4R (A_{—}23_P50121), FKSG83 (A_{—}23_P300080), KRTAP4-4 (A_{—}23_P38603), MPP5 (A_{—}23_P99536), (A_{—}23_P85874), BCAP29 (A_{—}23_P412526), PABPC1 (A_{—}23_P82693), CBR4 (A_{—}23_P213237), PTPDC1 (A_{—}23_P20876), CR12 (A_{—}23_P365844), FLJ20530 (A_{—}23_P43079), ALG8 (A_{—}23_P13554), CNOT6L (A_{—}23_P110304), KIF5B (A_{—}23_P86403), AGPAT5 (A_{—}23_P216200), CDCA7 (A_{—}23_P251421), FLJ14100 (A_{—}23_P121), IPO4 (A_{—}23_P162970), TMLHE (A_{—}23_P217546), ZNF527 (A_{—}23_P101932), OXA1L (A_{—}23_P2998), (A_{—}23_P118530), SIAT8D (A_{—}23_P41693), PTER (A_{—}23_P323774), T3JAM (A_{—}23_P201227), OR4A5 (A_{—}23_P98699), ZNF189 (A_{—}23_P216869), COPS4 (A_{—}23_P43779), NYD-SP21 (A_{—}23_P98561), Sep-10 (A_{—}23_P91168), IFNG (A_{—}23_P151294), TBR1 (A_{—}23_P5686), C20orf52 (A_{—}23_P143417), P1SRS (A_{—}23_P55641), HMGA1 (A_{—}23_P42331), NARG1L (A_{—}23_P14204), POFUT2 (A_{—}23_P211227), (A_{—}23_P30509), CLEC1 (A_{—}23_P204669), (A_{—}23_P209251), PPP1R1SA (A_{—}23_P90172), TADA2L (A_{—}23_P66664), LDHD (A_{—}23_P54918), NMUR2 (A_{—}23_P122134), LOC51035 (A_{—}23_P98605), GM2A (A_{—}23_P144872), (A_{—}23_P69931), WDR20 (A_{—}23_P205256), EXOSC4 (A_{—}23_P32463), ARHGEF7 (A_{—}23_P105845), SCEL (A_{—}23_P500000), (A_{—}23_P95619), BAG2 (A_{—}23_P255363), BACH1 (A_{—}23_P211047), OR8K3 (A_{—}23_P36050), CFL2 (A_{—}23_P65401), STK32B (A_{—}23_P21519), C7orf2 (A_{—}23_P300728), APRIN (A_{—}23_P205098), SLC2A12 (A_{—}23_P8279), TWSG1 (A_{—}23_P27256), TEX264 (A_{—}23_P41005), TATDN1 (A_{—}23_P254974), FAM11B (A_{—}23_P131176), PAK2 (A_{—}23_P106441), BIVM (A_{—}23_P105833)
(e) LOC132671 (A_{—}23_P407112), PTPN13 (A_{—}23_P18493), F2R (A_{—}23_P213562), PPIC (A_{—}23_P84018), FAM46A (A_{—}23_P70660), LHX6 (A_{—}23_P32175), TTC3 (A_{—}23_P120703), C5orf13 (A_{—}23_P92794), TPST1 (A_{—}23_P145965), FLJ23577 (A_{—}23_P252388), PGM1 (A_{—}23_P52031), SERTAD4 (A_{—}23_P318904), PXMP2 (A_{—}23_P135517), EPHA1 (A_{—}23_P157330), MYO5C (A_{—}23_P140434), DNER (A_{—}23_P362148), POLR2A (A_{—}23_P141235), IF (A_{—}23_P7212), KIAA0182 (A_{—}23_P152415), EDG2 (A_{—}23_P216609), ASRGL1 (A_{—}23_P203391), PLEKHE1 (A_{—}23_P89762), CROP (A_{—}23_P207493), ZFP36 (A_{—}23_P39237), DNAJC1 (A_{—}23_P127128), PXMP2 (A_{—}23_P124122), IPO7 (A_{—}23_P150714), TRA1 (A_{—}23_P2601), APOC3 (A_{—}23_P203183), TUBB2 (A_{—}23_P19291), SLC22A18 (A_{—}23_P139260), PLEKHK1 (A_{—}23_P52410), CD24 (A_{—}23_P114457), ZIC2 (A_{—}23_P36972), FTHL17 (A_{—}23_P148410), DUSP23 (A_{—}23_P103232), DPP7 (A_{—}23_P21574), AGXT2L1 (A_{—}23_P257231), ROR2 (A_{—}23_P158318), MAN2A1 (A_{—}23_P360769), HOXB7 (A_{—}23_P55276), FLJ20315 (A_{—}23_P3934), PPP1R14C (A_{—}23_P45011), CGI-115 (A_{—}23_P23356), EPLIN (A_{—}23_P151267), DIRC1 (A_{—}23_P131139), DPP3 (A_{—}23_P116091), CACNB2 (A_{—}23_P326852), SPDEF (A_{—}23_P111194), HDAC2 (A_{—}23_P122304), MSH3 (A_{—}23_P122001), EGR2 (A_{—}23_P46936), IL28RA (A_{—}23_P74112), SMPDL3A (A_{—}23_P72117), CAV1 (A_{—}23_P134454), LGALS3BP (A_{—}23_P15353), HCP5 (A_{—}23_P111125), OSBPL6 (A_{—}23_P108823), HOXB5 (A_{—}23_P363316), CLTB (A_{—}23_P43742), FLJ20054 (A_{—}23_P320897), KLF9 (A_{—}23_P415-401), STYK1 (A_{—}23_P13822), NR4A1 (A_{—}23_P128230), TACSTD1 (A_{—}23_P91081), ZNF608 (A_{—}23_P169978), FTH1 (A_{—}23_P87199), YPEL3 (A_{—}23_P15104), TM4SF9 (A_{—}23_P61886), HSPH1 (A_{—}23_P88119), ARL4A (A_{—}23_P145760), SLC1A1 (A_{—}23_P216468), CELSR1 (A_{—}23_P132378), RICS (A_{—}23_P161686), ITM2B (A_{—}23_P139934), PDE9A (A_{—}23_P29096), SHOX2 (A_{—}23_P112974), JAG1 (A_{—}23_P210763), DCP1A (A_{—}23_P166826), RIF1 (A_{—}23_P160689), EIF3S6 (A_{—}23_P154526), CLTB (A_{—}23_P252677), NNT (A_{—}23_P70148), PRKAR2B (A_{—}23_P42975), ZNF467 (A_{—}23_P59470), ID3 (A_{—}23_P137381), SEMA3C (A_{—}23_P256473), MLF1 (A_{—}23_P143906), NMU (A_{—}23_P69537), CHN1 (A_{—}23_P79482), TPD52L1 (A_{—}23_P31143), LAD1 (A_{—}23_P415510), DSC3 (A_{—}23_P208029), GJB2 (A_{—}23_P204947), PPP3CA (A_{—}23_P92623), SE57-1 (A_{—}23_P363255), VSNL1 (A_{—}23_P209978), PLOD2 (A_{—}23_P211910), EPM2AIP1 (A_{—}23_P415622), SCOC (A_{—}23_P167291), PDE8B (A_{—}23_P259065), ID2 (A_{—}23_P143143), BMP4 (A_{—}23_P54140), (A_{—}23_P253896), FGF2 (A_{—}23_P218918), RDX (A_{—}23_P203027),
(f) NR2F2 (A_{—}23_P88589), (A_{—}23_P101121), LOC129293 (A_{—}23_P255897), EML1 (A_{—}23_P205746), CDKN2A (A_{—}23_P250888), NCR1 (A_{—}23_P108042), ZNF586 (A_{—}23_P107693), (A_{—}23_P75585), FKBP9 (A_{—}23_P334709), GUCA1B (A_{—}23_P81825), PTGER4 (A_{—}23_P148047), CREB1 (A_{—}23_P79231), TFRC (A_{—}23_P212617), TMSB10 (A_{—}23_P68102), KLF7 (A_{—}23_P67977), C14orf145 (A_{—}23_P430201), NAG8 (A_{—}23_P250097), IL27 (A_{—}23_P315320), AFP (A_{—}23_P58201), DHX57 (A_{—}23_P28307), FBXW10 (A_{—}23_P218358), CRYAA (A_{—}23_P306755), HPCA (A_{—}23_P126162), CHST12 (A_{—}23_P310421), LOC340529 (A_{—}23_P159897), C14orf159 (A_{—}23_P48771), DHRS2 (A_{—}23_P48570), NFE2 (A_{—}23_P13753), PIK3C2B (A_{—}23_P200710), PSORS1C1 (A_{—}23_P133900), RAPGEF2 (A_{—}23_P133095), KIAA1797 (A_{—}23_P21673), EIF4EBP2 (A_{—}23_P115922), KLF6 (A_{—}23_P63798), S100A4 (A_{—}23_P94800), (A_{—}23_P164368), ZBTB10 (A_{—}23_P385114), SPRR2A (A_{—}23_P148949), TFAP2C (A_{—}23_P120472), C1orf24 (A_{—}23_P421326), KIAA1333 (A_{—}23_P99604), PPP1R14C (A_{—}23_P257617), MUM1 (A_{—}23_P400217), BMP2 (A_{—}23_P143324), PAPLN (A_{—}23_P140347), ARL61P (A_{—}23_P118142), ARID3A (A_{—}23_P16516), FANCA (A_{—}23_P206441), KIAA0040 (A_{—}23_P51327), PPIC (A_{—}23_P422724), FLJ23311 (A_{—}23_P35871), DYM (A_{—}23_P170518), BSCL2 (A_{—}23_P150566), ARGBP2 (A_{—}23_P121795), DSC2 (A_{—}23_P4494), DPP7 (A_{—}23_P72830), EFNA1 (A_{—}23_P254512), MGC10911 (A_{—}23_P168637), MFGE8 (A_{—}23_P48951), DLAT (A_{—}23_P203030), KIAA1468 (A_{—}23_P356125), KIAA0907 (A_{—}23_P74458), C11orf8 (A_{—}23_P52888), FLJ10156 (A_{—}23_P49878), MGC4308 (A_{—}23_P359174), KIAA1598 (A_{—}23_P202587), RANBP2L1 (A_{—}23_P384090), (A_{—}23_P200843), HMGB1 (A_{—}23_P99985), ANKHD1 (A_{—}23_P58443), ZNF92 (A_{—}23_P501080), PIB5PA (A_{—}23_P91669), ZIC3 (A_{—}23_P327910), BTBD11 (A_{—}23_P419712), DJ971N18.2 (A_{—}23_P166100), CHES1 (A_{—}23_P140405), THBD (A_{—}23_P91390), ASGR2 (A_{—}23_P130116), FLJ10858 (A_{—}23_P155711), NDOR1 (A_{—}23_P257407), CD164 (A_{—}23_P254756), GLTSCR1 (A_{—}23_P130773), RRBP1 (A_{—}23_P120566), RANBP2L1 (A_{—}23_P218637), DKFZp762A217 (A_{—}23_P382705), MYO9A (A_{—}23_P205841), CASP7 (A_{—}23_P12572), GPRC5B (A_{—}23_P324327), PP2447 (A_{—}23_P166553), FLRT1 (A_{—}23_P47168), SFRP5 (A_{—}23_P1355), CBX3 (A_{—}23_P31315), EIF5B (A_{—}23_P218608), GRIN2D (A_{—}23_P153549), HNRPH1 (A_{—}23_P252991), PPAP2A (A_{—}23_P70060), LOC284361 (A_{—}23_P208674), USP53 (A_{—}23_P167338), BICD2 (A_{—}23_P157751), UBE2E2 (A_{—}23_P10807), GLCCI1 (A_{—}23_P82402), CGI-143 (A_{—}23_P46507), FECH (A_{—}23_P89789), MBNL3 (A_{—}23_P96205), PTPRG (A_{—}23_P41054), FRMPD2 (A_{—}23_P86710), LCE1D (A_{—}23_P375524), RNF34 (A_{—}23_P128396), ITGB5 (A_{—}23_P166627), (A_{—}23_P206741), BIRC5 (A_{—}23_P1118815), LMNB1 (A_{—}23_P258493), (A_{—}23_P44235), TM4SF5 (A_{—}23_P27107), PTEN (A_{—}23_P98085), ACAS2L (A_{—}23_P120594), HNRPH2 (A_{—}23_P 11283), CALM3 (A_{—}23_P4944), H2AFV (A_{—}23_P145904), RACGAP1 (A_{—}23_P65110), FLJ20160 (A_{—}23_P28530), MGC13204 (A_{—}23_P105583), VPS35 (A_{—}23_P66149), FLJ37078 (A_{—}23_P331700), PPARBP (A_{—}23_P425704), ASAM (A_{—}23_P35995), NPC1L1 (A_{—}23_P20075), SUCNR1 (A_{—}23_P69171), EPHA8 (A_{—}23_P103199), (A_{—}23_P16562), C14orf24 (A_{—}23_P2935), SLC30A3 (A_{—}23_P302568), SGEF (A_{—}23_P69100), MIDN (A_{—}23_P153709), RPN1 (A_{—}23_P132803), PSPC1 (A_{—}23_P76610), MGC39633 (A_{—}23_P92765), ZFOC1 (A_{—}23_P359654), MAT2A (A23—P401568), (A_{—}23_P353541), FREM1 (A_{—}23_P43334), ZNF449 (A_{—}23_P309865), KLHL7 (A_{—}23_P324994), SRRM1 (A_{—}23_P23803), GYPA (A_{—}23_P212854), FLJ21827 (A_{—}23_P116202), THEA (A_{—}23_P417-415), NICN1 (A_{—}23_P110005), UNQ1912 (A_{—}23_P30283), LRRC1 (A_{—}23_P215024), PLSCR4 (A_{—}23_P91912), PP1665 (A_{—}23_P87401), ELF2 (A_{—}23_P132948), AZI2 (A_{—}23_P109682), CBLB (A_{—}23_P212715), IFNK (A_{—}23_P310372), GNA14 (A_{—}23_P415561), (A_{—}23_P341418), TNRC6C (A_{—}23_P392501), MCM3 (A_{—}23_P7873), NDE1 (A_{—}23_P206901), RHBG (A_{—}23_P51690), CABYR (A_{—}23_P314712), MFN2 (A_{—}23_P126135), PHF13 (A_{—}23_P384559), SCD4 (A_{—}23_P213288), (A_{—}23_P167432), ET (A_{—}23_P26704), (A_{—}23_P65040), IQCC (A_{—}23_P74162), LCN7 (A_{—}23_P85585), TBC1D16 (A_{—}23_P428326), DNM1DN8-2 (A_{—}23_P106472), METRNL (A_{—}23_P10591), FBXO17 (A_{—}23_P101875), FTO (A_{—}23_P113184), NDUFS4 (A_{—}23_P257198), (A_{—}23_P259103), TRIM40 (A_{—}23_P402778), C20orf179 (A_{—}23_P330618), ACRBP (A_{—}23_P151120), UGT2B7 (A_{—}23_P136671), ZNF131 (A_{—}23_P133315), FLJ22353 (A_{—}23_P85853), DIAPH2 (A_{—}23_P113172), NOLC1 (A_{—}23_P314151), NUSAP1 (A_{—}23_P206183), HT017 (A_{—}23_P40856), MKL2 (A_{—}23_P54556), TRA2A (A_{—}23_P31386), PRC1 (A_{—}23_P206059), GOSR1 (A_{—}23_P252641), WIRE (A_{—}23_P366454), LMLN (A_{—}23_P43786), ART5 (A_{—}23_P427119), HLA-DQB2 (A_{—}23_P 19510), ADK (A_{—}23_P112596), RNF26 (A_{—}23_P64630), COPG (A_{—}23_P44617), (A_{—}23_P26311), BAT2 (A_{—}23_P30870), (A_{—}23_P141263), DSCR4 (A_{—}23_P40455), RLBP1 (A_{—}23_P163361), TFCP2 (A_{—}23_P347528), AEGP (A_{—}23_P71787), ZNF365 (A_{—}23_P86610), PHOX2B (A_{—}23_P213228), ARPP-19 (A_{—}23_P205768), WNT10B (A_{—}23_P162317), AGRP (A_{—}23_P502320), PPP1R9B (A_{—}23_P55242), P4HA1 (A_{—}23_P35521), HDAC1 (A_{—}23_P 114656), CENTA2 (A_{—}23_P49816), PIN4 (A_{—}23_P315345), (A_{—}23_P72949), CHSY1 (A_{—}23_P37484), FTSJ2 (A_{—}23_P31253), RFP (A_{—}23_P42224), TAPBPL (A_{—}23_P36698), ADSSL1 (A_{—}23_P76823), FLJ31153 (A_{—}23_P390744), LRP1B (A_{—}23_P5342), BRD1 (A_{—}23_P166536), QTRTD1 (A_{—}23_P91930), (A_{—}23_P18023), FLJ25555 (A_{—}23_P380881), (A_{—}23_P70998), SYNJ2 (A_{—}23_P316974), (A_{—}23_P167624), ALS2CR3 (A_{—}23_P209426), FLJ34443 (A_{—}23_P431330), SOX4 (A_{—}23_P82176), VDR (A_{—}23_P162589), FBXL2 (A_{—}23_P17955), D4S234E (A_{—}23_P371106), CXorf6 (A_{—}23_P251075), KIF24 (A_{—}23_P43595), ETV7 (A_{—}23_P42347), FLJ14721 (A_{—}23_P128375), MGC13168 (A_{—}23_P150960), CAP350 (A_{—}23_P201816), BCAS1 (A_{—}23_P17420), IL4R (A_{—}23_P129556), CTAG2 (A_{—}23_P22693), CDV1 (A_{—}23_P72680), RBM15 (A_{—}23_P34879), MGC34923 (A_{—}23_P91850), ING2 (A_{—}23_P125687), BTBD11 (A_{—}23_P99349), SUV420H1 (A_{—}23_P217968), (A_{—}23_P158925), CTSD (A23—P52556), C1QTNF3 (A_{—}23_P122068), MTA1 (A_{—}23_P158129), TNK2 (A_{—}23_P259846), C21orf81 (A_{—}23_P392529), MTCP1 (A_{—}23_P11295), TDRD3 (A_{—}23_P2711), (A_{—}23_P253774), (A_{—}23_P139725), USP10 (A_{—}23_P100196), BCL3 (A_{—}23_P4662), GALNS (A_{—}23_P106562), DIRAS2 (A_{—}23_P379778), KIAA0467 (A_{—}23_P51639), RHOU (A_{—}23_P114814), (A_{—}23_P50297), (A_{—}23_P252175), FLJ37099 (A_{—}23_P46315), MFHAS1 (A_{—}23_P112078), GAGED4 (A_{—}23_P114343), PIK3CB (A_{—}23_P92253), ECT2 (A_{—}23_P9571), MAP3K71P1 (A_{—}23_P80345), (A_{—}23_P110557), ASB12 (A_{—}23_P96395), C1QTNF6 (A_{—}23_P57513), FGF12 (A_{—}23_P169553), CNTN3 (A_{—}23_P250173), LOC150678 (A_{—}23_P377212), SUPT3H (A_{—}23_P251621), C19orf14 (A_{—}23_P339705), PRKCZ (A_{—}23_P51187), SEMA5A (A_{—}23_P213415), TRIM6 (A_{—}23_P33675), CIDEA (A_{—}23_P258592), ATR (A_{—}23_P124664), HIST1H2AC (A_{—}23_P372860), DIO2 (A_{—}23_P48740), FBXL18 (A_{—}23_P423957), DKFZP434HO115 (A_{—}23_P73150), CTSO (A_{—}23_P110175),
(g) CYorf15B (A_{—}23_P96652), MTUS1 (A_{—}23_P94358), RBPMS2 (A_{—}23_P100059), DKK1 (A_{—}23_P24129), KCNMB4 (A_{—}23_P64792), NALP2 (A_{—}23_P130824), EMILIN2 (A_{—}23_P27315), (A_{—}23_P14216), PRKG1 (A_{—}23_P136041), SERPINE2 (A_{—}23_P50919), FOXA1 (A_{—}23_P37127), SPINT2 (A_{—}23_P27795), PTPRK (A_{—}23_P254924), HSPC105 (A_{—}23_P129458), MCTP2 (A_{—}23_P65789), KBTBD7 (A_{—}23_P25605), DKFZp762K222 (A_{—}23_P251364), TBX1 (A_{—}23_P211345), COBLL1 (A_{—}23_P79289), DSP (A_{—}23_P31031), CCL28 (A_{—}23_P503072), TCF7L2 (A_{—}23_P127014), C3orf4 (A_{—}23_P155556), CTHRC1 (A_{—}23_P111888), ATP6V1C2 (A_{—}23_P250914), MTAC2D1 (A_{—}23_P413075), RBM24 (A_{—}23_P167812), FLJ20972 (A_{—}23_P366890), CLOCK (A_{—}23_P419038), KLRC3 (A_{—}23_P22232), MGC11242 (A_{—}23_P118894), HMMR (A_{—}23_P70007), FBLN5 (A_{—}23_P151805), SPINK2 (A_{—}23_P155688), RRAGD (A_{—}23_P133684), CCL2 (A_{—}23_P89431), PSCD1 (A_{—}23_P95184), PDZRN3 (A_{—}23_P21618), MYOM2 (A_{—}23_P258912), UGP2 (A_{—}23_P253046), IFITM1 (A_{—}23_P72737), PSTPIP2 (A_{—}23_P208119), CDH3 (A_{—}23_P49155), SASH1 (A_{—}23_P93442), HOXB6 (A_{—}23_P66682), PITX1 (A_{—}23_P58636), CYP27B1 (A_{—}23_P36397), STXBP6 (A_{—}23_P205713), PCK1 (A_{—}23_P408249), MYB (A_{—}23_P31073), DKFZP434P1750 (A_{—}23_P22382), TCF7L2 (A_{—}23_P328501), GALNT13 (A_{—}23_P165369), ME1 (A_{—}23_P8196), FZD3 (A_{—}23_P347471), ADD3 (A_{—}23_P202435), FLJ13391 (A_{—}23_P108676), TGFBR3 (A_{—}23_P200780), CYB5 (A_{—}23_P101208), (A_{—}23_P58697), KIAA0882 (A_{—}23_P41487), DNCLI2 (A_{—}23_P9688), GSDML (A_{—}23_P66454), SYTL2 (A_{—}23_P53193), PHGDH (A_{—}23_P85780), FZD7 (A_{—}23_P209449), GSTP1 (A_{—}23_P202658), HEYL (A_{—}23_P46245), BSN (A_{—}23_P29735), PIR (A_{—}23_P137035), CNTNAP2 (A_{—}23_P95802), C2orf23 (A_{—}23_P96285), IFITM3 (A_{—}23_P87539), 13CDNA73 (A_{—}23_P105862), UGDH (A_{—}23_P167067), HOXB8 (A_{—}23_P370586), LRP11 (A_{—}23_P19652), DLX4 (A_{—}23_P164196), SYNCRIP (A_{—}23_P214798), NCOA1 (A_{—}23_P39594), CD9 (A_{—}23_P76364), C6orf60 (A_{—}23_P167818), FGFR3 (A_{—}23_P500501), (A_{—}23_P35546), ERBB3 (A_{—}23_P349416), MAP3K5 (A_{—}23_P134125), IFRG28 (A_{—}23_P166797), SGCE (A_{—}23_P254626), LOC148418 (A_{—}23_P61438), GSTA1 (A_{—}23_P135417), MTCH1 (A_{—}23_P145388), OVOL1 (A_{—}23_P202810), PAH (A_{—}23_P2502), RBP7 (A_{—}23_P97431), ASS (A_{—}23_P31922), CAMTA2 (A_{—}23_P365189), FLJ30525 (A_{—}23_P309361), TM4SF9 (A_{—}23_P323930), EFHD1 (A_{—}23_P154338), CDH1 (A_{—}23_P206359), (A_{—}23_P138635), LPL (A_{—}23_P146233), RUNX3 (A_{—}23_P51231), PAG (A_{—}23_P347070), SLC40A1 (A_{—}23_P102391), SLC43A3 (A_{—}23_P358545), SLC12A2 (A_{—}23_P133606), MTAC2D1 (A_{—}23_P88439), CLDN4 (A_{—}23_P19944), MAP3K12 (A_{—}23_P105405), EPHB6 (A_{—}23_P145935), PERP (A_{—}23_P214950), ID1 (A_{—}23_P252306), MIDI (A_{—}23_P32838), FLJ10901 (A_{—}23_P1043), STX6 (A_{—}23_P97725), GABARAPL1 (A_{—}23_P65817), PAPSS2 (A_{—}23_P104492), PERP (A_{—}23_P369298), UBE1L (A_{—}23_P9809), EDG8 (A_{—}23_P107744), TGFB1 (A_{—}23_P412335), MIDI (A_{—}23_P170037), SMAD7 (A_{—}23_P55518), HYAL3 (A_{—}23_P212400), PRRX2 (A_{—}23_P83298), KITLG (A_{—}23_P204654), CPOX (A_{—}23_P144179), CARD15 (A_{—}23_P420863), AXIN2 (A_{—}23_P148014), ICK (A_{—}23_P214315), AXIN2 (A_{—}23_P159395), LIN7C (A_{—}23_P139277), GNAI1 (A_{—}23_P122976), LGALS3 (A_{—}23_P128918), ZFP36L2 (A_{—}23_P101960), LHX2 (A_{—}23_P32165), RECK (A_{—}23_P83028), CKB (A_{—}23_P25674), C6orf114 (A_{—}23_P134058), SNTB1 (A_{—}23_P95029), KLF5 (A_{—}23_P53891), DPP4 (A_{—}23_P39885), LOC146177 (A_{—}23_P129397), LAPTM4B (A_{—}23_P59926), (A_{—}23_P41664), PROM1 (A_{—}23_P258462), MPP1 (A_{—}23_P171296), KCNS3 (A_{—}23_P120105), PDGFC (A_{—}23_P58396), HEPH (A_{—}23_P22526), CHAT (A_{—}23_P46894), GAMT (A_{—}23_P108143), ELOVL5 (A_{—}23_P156498), KLHL13 (A_{—}23_P159974), MIDI (A_{—}23_P10031), FLJ1196 (A_{—}23_P117782), MAN1A1 (A_{—}23_P156431), MAOA (A_{—}23_P96410), DDX3Y (A_{—}23_P217797), SORL1 (A_{—}23_P87049), C1QL3 (A_{—}23_P 1186), MTERF (A_{—}23_P 111712),
(h) PLCE1 (A_{—}23_P35617), PTPRK (A_{—}23_P254242), KLRC1 (A_{—}23_P151046), PTPN12 (A_{—}23_P8763), ATP1B1 (A_{—}23_P62932), CGREF1 (A_{—}23_P210235), RHBDL6 (A_{—}23_P329870), BARD1 (A_{—}23_P67771), (A_{—}23_P61112), C14orf101 (A_{—}23_P205607), ZNF205 (A_{—}23_P3748), VPREB1 (A_{—}23_P29152), KIF12 (A_{—}23_P60550), CXCR4 (A_{—}23_P102000), CHST7 (A_{—}23_P319617), TARDBP (A_{—}23_P403955), CLDN23 (A_{—}23_P134854), MED12 (A_{—}23_P73699), IRF2BP2 (A_{—}23_P46588), CYP26A1 (A_{—}23_P138655), TJP2 (A_{—}23_P9293), SIPA1L2 (A_{—}23_P137470), GDF8 (A_{—}23_P165727), GPSM2 (A_{—}23_P63402), AES (A_{—}23_P165061), SLC6A14 (A_{—}23_P171038), FNBP1 (A_{—}23_P32249), KIAA0998 (A_{—}23_P 151756), SQRDL (A_{—}23_P3221), PCDH7 (A_{—}23_P212888), PTMA (A_{—}23_P434305), NOLC1 (A_{—}23_P202140), CXXC4 (A_{—}23_P121676), IPO7 (A_{—}23_P331598), TTC13 (A_{—}23_P103864), THBS1 (A_{—}23_P206212), MIDN (A_{—}23_P324461), RDHE2 (A_{—}23_P257457), USP43 (A_{—}23_P152696), BCLP (A_{—}23_P62768), FLJ33996 (A_{—}23_P399880), CDKN3 (A_{—}23_P48669), SEC31L2 (A_{—}23_P35564), HIST1H3F (A_{—}23_P30796), PLEKHA6 (A_{—}23_P431268), ENC1 (A_{—}23_P213424), POR (A_{—}23_P19964), DPYSL5 (A_{—}23_P210224), MLH1 (A_{—}23_P69058), HIST1H2AK (A_{—}23_P42220), B7-H4 (A_{—}23_P518), DYRK4 (A_{—}23_P87899), GAB1 (A_{—}23_P335239), IGSF9 (A_{—}23_P85441), DPYSL3 (A_{—}23_P7528), GALNT12 (A_{—}23_P415652), C10orf78 (A_{—}23_P413193), CDW92 (A_{—}23_P216630), ACVR1C (A_{—}23_P67820), GABARAPL1 (A_{—}23_P162640), EGFL9 (A_{—}23_P133786), CCR2 (A_{—}23_P212354), MYOD1 (A_{—}23_P53033), CR2 (A_{—}23_P124542), DAZAP2 (A_{—}23_P40025), C6orf85 (A_{—}23_P7882), ERP70 (A_{—}23_P42800), DST (A_{—}23_P59388), SLC35E2 (A_{—}23_P61860), FZD3 (A_{—}23_P215922), SMCY (A_{—}23_P137238), MMP11 (A_{—}23_P57417), LCE1C (A_{—}23_P114607), MESDC1 (A_{—}23_P99891), (A_{—}23_P20168), HSD17B4 (A_{—}23_P92954), BDKRB2 (A_{—}23_P304897), GALNT12 (A_{—}23_P257731), ARK5 (A_{—}23_P348257), LUZP4 (A_{—}23_P96611), SYP (A_{—}23_P136935), LHFP (A_{—}23_P88069), CSTB (A_{—}23_P154889), NR2F2 (A_{—}23_P422703), CUL5 (A_{—}23_P203009), CLDN1 (A_{—}23_P57779), KIF20A (A_{—}23_P256956), UQCRC2 (A_{—}23_P118002), NDUFA9 (A_{—}23_P76499), GALNT1 (A_{—}23_P306105), DHRS1 (A_{—}23_P48747), MOSPD1 (A_{—}23_P73828), PMM2 (A_{—}23_P432360), EIF2AK1 (A_{—}23_P251173), LNX (A_{—}23_P213139), LTBP1 (A_{—}23_P43810), TM4SF2 (A_{—}23_P114185), CHST11 (A_{—}23_P139919), TIMP3 (A_{—}23_P211468), BMP7 (A_{—}23_P68488), (A_{—}23_P34007), HRIHFB2122 (A23—P 17854), ENPEP (A_{—}23_P 144596), C1S (A_{—}23_P2492), IDH2 (A_{—}23_P129204), RNASE4 (A_{—}23_P428738), SLC17A4 (A_{—}23_P357270), NRAP (A_{—}23_P402765), BTG3 (A_{—}23_P80068), MGC23401 (A_{—}23_P150950), C1orf34 (A_{—}23_P160214), ATP5G3 (A_{—}23_P56678), LOC92305 (A_{—}23_P354175), C6orf111 (A_{—}23_P122617), TBC1D8 (A_{—}23_P253281), MTMR8 (A_{—}23_P84995), (A_{—}23_P310900), UBA2 (A_{—}23_P209020), STK33 (A_{—}23_P127915), BCHE (A_{—}23_P212050), OGFR (A_{—}23_P28707), CITED2 (A_{—}23_P214969), KIAA1961 (A_{—}23_P144994), FLJ10707 (A_{—}23_P212204), (A_{—}23_P93311), GOSR1 (A_{—}23_P4373), PMAIP1 (A_{—}23_P207999), ADAM20 (A_{—}23_P48698), LAMB2 (A_{—}23_P21382), GTDC1 (A_{—}23_P153945), PDXK (A_{—}23_P68730), SGK (A_{—}23_P19673), LOC90637 (A_{—}23_P336992), SRP72 (A_{—}23_P337201), RAB31 (A_{—}23_P141688), SLC9A3R1 (A_{—}23_P152593), CTBP2 (A_{—}23_P63897), DLGAP3 (A_{—}23_P149707), SLC9A3R1 (A_{—}23_P308519), FLJ38973 (A_{—}23_P142916), RHOBTB1 (A_{—}23_P35634), ZCCHC2 (A_{—}23_P66958), KIAA1036 (A_{—}23_P76998) CTSL2 (A_{—}23_P146456), (A_{—}23_P111797), DBC1 (A_{—}23_P94517), MTMR1 (A_{—}23_P73530), C9orf55 (A_{—}23_P157726), SERPINF1 (A_{—}23_P100660), SH3KBP1 (A_{—}23_P374777), C9orf19 (A_{—}23_P71627), ANGPTL1 (A_{—}23_P126706), (A_{—}23_P64184), FKBP1B (A_{—}23_P142631), VAMP8 (A_{—}23_P28434), HOXD3 (A_{—}23_P79652), MBIP (A_{—}23_P2922), CD48 (A_{—}23_P74145), RHBDL1 (A_{—}23_P26468), MCC (A_{—}23_P377114), PMM2 (A_{—}23_P206937), FGD1 (A_{—}23_P73559), EIF4G2 (A_{—}23_P104892), TRADD (A_{—}23_P54649), CGN (A_{—}23_P149388), HCAP-G (A_{—}23_P155815), SMARCC2 (A_{—}23_P128073), CAPS2 (A_{—}23_P124773), NHLRC2 (A_{—}23_P46767), PC (A_{—}23_P161647), VRK2 (A_{—}23_P119992), OR3A1 (A_{—}23_P50031), GRP58 (A_{—}23_P99883), FXR1 (A_{—}23_P132784), MMRN2 (A_{—}23_P150057), PON1 (A_{—}23_P168598), MCMDC1 (A_{—}23_P397347), ZA20D2 (A_{—}23_P71752), KIRREL2 (A_{—}23_P16583), SFRS2 (A_{—}23_P77776), MRPS7 (A_{—}23_P3973), HS6ST1 (A_{—}23_P90579), (A_{—}23_P258144), ZF (A_{—}23_P203649), MARCH-IX (A_{—}23_P47777), PPARA (A_{—}23_P40724), SESN3 (A_{—}23_P361448), KPNA2 (A_{—}23_P125265), RGC32 (A_{—}23_P204937), SYT1 (A_{—}23_P36795), (A_{—}23_P150996), SS18 (A_{—}23_P141738), ITIH2 (A_{—}23_P202053), C7orf24 (A_{—}23_P42695), (A_{—}23_P 169828), SP 100 (A_{—}23_P349928), LOC90379 (A_{—}23_P50399), CAMK4 (A_{—}23_P250347), USP52 (A_{—}23_P64689), PTMA (A_{—}23_P210283), TUBB4Q (A_{—}23_P140884), (A_{—}23_P166280), KIAA1536 (A_{—}23_P98995), LRP2 (A_{—}23_P28295), A4GALT (A_{—}23_P57568), C13orf11 (A_{—}23_P205188), HIBADH (A_{—}23_P168507), (A_{—}23_P15226), USP8 (A_{—}23_P129053), VPS41 (A_{—}23_P215318), PCP4 (A_{—}23_P109322), PJA2 (A_{—}23_P133470), HMGB1 (A_{—}23_P162805), NOS3 (A_{—}23_P70849), ROCK2 (A_{—}23_P209689), NEBL (A_{—}23_P104522), ADORA1 (A_{—}23_P74299), HEXA (A_{—}23_P129096), ZNF573 (A_{—}23_P339079), ABCA5 (A_{—}23_P78018), (A_{—}23_P104781), NXN (A_{—}23_P61778), FLJ14166 (A_{—}23_P7684), RYR3 (A_{—}23_P94838), NJMU-R1 (A_{—}23_P15639), FLJ13213 (A_{—}23_P26094), ING1 (A_{—}23_P99437), C2orf15 (A_{—}23_P415511), NEXN (A_{—}23_P200001), TBC1D3 (A_{—}23_P78068), HLCS (A_{—}23_P304991), (A_{—}23_P256329), SAFB2 (A_{—}23_P27894), CACNB3 (A_{—}23_P204019), CNTN1 (A_{—}23_P204541), CRLF1 (A_{—}23_P56197), CPNE4 (A_{—}23_P327551), COPZ1 (A_{—}23_P116802), Sep-09 (A_{—}23_P106973), PDGFRL (A_{—}23_P60148), C14orf129 (A_{—}23_P205336), RALA (A_{—}23_P215302), LOC90799 (A_{—}23_P307400), TMEM29 (A_{—}23_P148519), FLJ10378 (A_{—}23_P7253), HOXB9 (A_{—}23_P27013), MARK1 (A_{—}23_P424), CSNK1G1 (A_{—}23_P83704), FLJ20674 (A_{—}23_P72058), MPHOSPH1 (A_{—}23_P75071), SMARCA3 (A_{—}23_P57676), PROCA1 (A_{—}23_P427148), DHRS8 (A_{—}23_P408271), FANCL (A_{—}23_P131383), DHRSX (A_{—}23_P251339), STXBP2 (A_{—}23_P130614), ACO2 (A_{—}23_P103149), FLJ33069 (A_{—}23_P321351), FGFRL1 (A_{—}23_P92349), GALNT1 (A_{—}23_P153137), ADH1B (A_{—}23_P213184), EPSTI1 (A_{—}23_P105794), PCNT2 (A_{—}23_P57347), RNF165 (A_{—}23_P371280), HBZ (A_{—}23_P3651), LRRC4 (A_{—}23_P8625), OR5M9 (A_{—}23_P104939), PLGL (A_{—}23_P359630), GEFT (A_{—}23_P98844), PRRX1 (A_{—}23_P502731), EZH2 (A_{—}23_P259641), CHD3 (A_{—}23_P21640), SERPINA10 (A_{—}23_P128759), CGI-111 (A_{—}23_P250982), KIAA1447 (A_{—}23_P49539), FLJ13231 (A_{—}23_P213877), (A_{—}23_P146744), C1orf38 (A_{—}23_P873), ETV5 (A_{—}23_P9831), SUCLA2 (A_{—}23_P 117157), RNF44 (A_{—}23_P213592), PPM ID (A_{—}23_P89349), HDHD2 (A_{—}23_P153098), ITIH3 (A_{—}23_P6821), PCP2 (A_{—}23_P355860), C6orf210 (A_{—}23_P134167), C22orf24 (A_{—}23_P166431), FNBP1L (A_{—}23_P417942), TYMS (A_{—}23_P50096), ZMPSTE24 (A_{—}23_P137427), BTNL2 (A_{—}23_P376686), PLVAP (A_{—}23_P56328), DMXL1 (A_{—}23_P250571), CHES1 (A_{—}23_P88434), SRGAP1 (A_{—}23_P162446), RPIP8 (A_{—}23_P66579), ATP2B1 (A_{—}23_P128319), FCGR2B (A_{—}23_P34650), RCN1 (A_{—}23_P203299), FLJ10330 (A_{—}23_P201565), TNFAIP3 (A_{—}23_P156898), FLJ23191 (A_{—}23_P110266), GLG1 (A_{—}23_P206510), CCDC7 (A_{—}23_P374294), NBEA (A_{—}23_P21128), PB1 (A_{—}23_P218863), PAG (A_{—}23_P215841), ENPEP (A_{—}23_P333605), FLJ14281 (A_{—}23_P213199), CTAGE5 (A_{—}23_P128773), FLJ23467 (A_{—}23_P46355), IL18R1 (A_{—}23_P39735), RBM19 (A_{—}23_P204119), IK (A_{—}23_P133245), SC5DL (A_{—}23_P372888), LOC 115509 (A_{—}23_P129659), CYYR1 (A_{—}23_P 17620), ZNF589 (A_{—}23_P 110031), ELOVL2 (A_{—}23_P251606), KIF23 (A_{—}23_P48835), PDE6A (A_{—}23_P81588), NDP52 (A_{—}23_P4221), IRX3 (A_{—}23_P152237), COX7C (A_{—}23_P110811), APEG1 (A_{—}23_P338919), CHRDL2 (A_{—}23_P127990), MYST3 (A_{—}23_P407628), C11orf15 (A_{—}23_P162087), HMGB3 (A_{—}23_P217236), ANKRD25 (A_{—}23_P50426), (A_{—}23_P218523), BAZ1B (A23—P215449), FLJ23033 (A_{—}23_P97481), (A_{—}23_P61937), ASK (A_{—}23_P254612), AK5 (A_{—}23_P200015), LOC147650 (A_{—}23_P27392), C10orf3 (A_{—}23_P115872), FSTL3 (A_{—}23_P209160), ARMET (A_{—}23_P132793), C18orf22 (A_{—}23_P153086), C6orf62 (A_{—}23_P422222), MYH2 (A_{—}23_P38271), SEC61G (A_{—}23_P71241), ZNF31 (A_{—}23_P23102), H2AFY (A_{—}23_P70045), MESDC2 (A_{—}23_P88503), MBNL1 (A_{—}23_P357811), HCN3 (A_{—}23_P34827), (A_{—}23_P203228), (A_{—}23_P125602), DERP6 (A_{—}23_P164289), LUC7L2 (A_{—}23_P59790), MUC15 (A_{—}23_P24332), NDUFV1 (A_{—}23_P127353), YWHAH (A_{—}23_P103070), RNF103 (A_{—}23_P56709), MAN2A2 (A_{—}23_P37564), (A_{—}23_P41340), CLDN11 (A_{—}23_P29800), RNF133 (A_{—}23_P42963), UBASH3A (A_{—}23_P6293), PB1 (A_{—}23_P365874), CDK5RAP3 (A_{—}23_P66900), Sep-03 (A_{—}23_P211572), (A_{—}23_P71179), CALML5 (A_{—}23_P43841), NEK2 (A_{—}23_P35219), EME1 (A_{—}23_P368225), USMG5 (A_{—}23_P127095), BM039 (A_{—}23_P88740), LBX1 (A_{—}23_P86493), GNG4 (A_{—}23_P335329), (A_{—}23_P45712), DLG7 (A_{—}23_P88331), GPRC5C (A_{—}23_P38167), APOH (A_{—}23_P38244), APBB3 (A_{—}23_P110445), ATP6VOA2 (A_{—}23_P87513), (A_{—}23_P30055), LCE3E (A_{—}23_P340340), FLJ23506 (A_{—}23_P50217), CXXC5 (A_{—}23_P213680), RAB10 (A_{—}23_P165879), HOZFP (A_{—}23_P407090), RABGAP1L (A_{—}23_P377264), MARCO (A_{—}23_P101992), OIP5 (A_{—}23_P379614), PELP1 (A_{—}23_P49898), PPY (A_{—}23_P207336), EFNB2 (A_{—}23_P428139), PPIA (A_{—}23_P258340), C20orf100 (A_{—}23_P154566), POPDC3 (A_{—}23_P358599), PPFIA1 (A_{—}23_P75509), ZNF652 (A_{—}23_P55256), MGC10334 (A_{—}23_P63281), SC5DL (A_{—}23_P98446), OVOL1 (A_{—}23_P326700), THOC2 (A_{—}23_P11051), DKFZP566D1346 (A_{—}23_P114616), MOCS1 (A_{—}23_P58993), RAD17 (A_{—}23_P 159053), MRPL48 (A_{—}23_P 162106), (A_{—}23_P54406), SP 110 (A_{—}23_P 120002), DTX3L (A_{—}23_P347040), MYLC2PL (A_{—}23_P393015), DMPK (A_{—}23_P50535), KIAA1279 (A_{—}23_P380815), FLJ23754 (A_{—}23_P365117), DGKD (A_{—}23_P210253), GCLM (A_{—}23_P103996), SLC1A7 (A_{—}23_P325562), RBBP8 (A_{—}23_P252371), KCNJ14 (A_{—}23_P130764), PDPR (A_{—}23_P206677), FRMD1 (A_{—}23_P81717), NUCB2 (A_{—}23_P13364), SS18L2 (A_{—}23_P166698), FLJ31713 (A_{—}23_P354326), TDRD4 (A_{—}23_P99373), S100A8 (A_{—}23_P200288), (A_{—}23_P42514), APOL4 (A_{—}23_P211479), HECTD2 (A_{—}23_P355525), (A_{—}23_P133257), ZCCHC11 (A_{—}23_P34433), LMAN2 (A_{—}23_P169599), (A_{—}23_P131036), KIAA0372 (A_{—}23_P61854), OSBPL5 (A_{—}23_P53081), CBX1 (A_{—}23_P107509), MOGAT2 (A_{—}23_P203692), FASN (A_{—}23_P44132), DKFZp762E1312 (A_{—}23_P79429), PTPRA (A_{—}23_P28869), RYR2 (A_{—}23_P137797), PDPK1 (A_{—}23_P66219), DAPP1 (A_{—}23_P255444), TNRC6A (A_{—}23_P349310), KRAS2 (A_{—}23_P45140), TRIM15 (A_{—}23_P214554), RCOR2 (A_{—}23_P24397), C2orf22 (A_{—}23_P131375), TFDP1 (A_{—}23_P14243), RFC5 (A_{—}23_P95302), STAT1 (A_{—}23_P56630), MYBPH (A_{—}23_P148737), PCCA (A_{—}23_P48358), PSG9 (A_{—}23_P39309), ASPM (A_{—}23_P52017), (A_{—}23_P109676), DEGS2 (A_{—}23_P88450), FLJ32112 (A_{—}23_P167738), FLJ25179 (A_{—}23_P372962), GP1BB (A_{—}23_P29124), PRPF39 (A_{—}23_P163107), COL4A1 (A_{—}23_P65240), C1QTNF2 (A_{—}23_P92899), TFCP2L2 (A_{—}23_P5882), MTA1 (A_{—}23_P9513), PHF20 (A_{—}23_P428835), FLJ23342 (A_{—}23_P64280), R3HDM (A_{—}23_P380998), REC8L1 (A_{—}23_P322550), ACCN4 (A_{—}23_P56508), AIM1L (A_{—}23_P360324), SLCO1B1 (A_{—}23_P128254), (A_{—}23_P135837), TAC3 (A_{—}23_P2283), (A_{—}23_P73820), KLRC4 (A_{—}23_P218058), EP400 (A_{—}23_P253154), FLJ25955 (A_{—}23_P28466), GIP (A_{—}23_P141459), PSMA3 (A_{—}23_P140301), PPP2R5E (A_{—}23_P3042), P53AIP1 (A_{—}23_P340171), FLJ40873 (A_{—}23_P367014), PTPRJ (A_{—}23_P405048), FLJ39739 (A_{—}23_P74993), MAD1L1 (A_{—}23_P42657), C9orf39 (A_{—}23_P157963), EIF2B2 (A_{—}23_P25926), PIP5K2B (A_{—}23_P54983), MAML1 (A_{—}23_P110604), NRN1 (A_{—}23_P82088), Cep164 (A_{—}23_P75609), FLRT3 (A_{—}23_P166109), PAI-RBP1 (A_{—}23_P200661), C13orf6 (A_{—}23_P205027), ARID4B (A_{—}23_P201951), MKI67 (A_{—}23_P202232), LOC388152 (A_{—}23_P129103), UNG (A_{—}23_P76388), (A_{—}23_P63644), C1GALT1 (A_{—}23_P252145), RAB2 (A_{—}23_P253949), SDF2L1 (A_{—}23_P6344), GHR (A_{—}23_P 156087), IGLC2 (A_{—}23_P72271), C21 orf2 (A_{—}23_P211167), LY6G6C (A_{—}23_P8083), (A_{—}23_P20573), HSPA2 (A_{—}23_P88303), NPB (A_{—}23_P38545), C21orf91 (A_{—}23_P211015), KIAA0355 (A_{—}23_P324490), SLC17A6 (A_{—}23_P24294), BDKRB2 (A_{—}23_P140142), CDC5L (A_{—}23_P156471), ATF71P2 (A_{—}23_P129466), ABCB10 (A_{—}23_P201918), NOSTRIN (A_{—}23_P79360), FLJ1000 (A_{—}23_P31176), LMTK2 (A_{—}23_P410746), LOC124773 (A_{—}23_P164100), (A_{—}23_P255091), SP192 (A_{—}23_P593), ZFR (A_{—}23_P41818), OR51E2 (A_{—}23_P139327), TP53TG3 (A_{—}23_P37994), EIF2B1 (A_{—}23_P105313), PTPRCAP (A_{—}23_P98173), LIMR (A_{—}23_P150931), ELAC1 (A_{—}23_P15942), YWHAG (A_{—}23_P123022), (A_{—}23_P42565), ADAM11 (A_{—}23_P502158), LYZ (A_{—}23_P76192), ADK (A_{—}23_P125333), MSCP (A_{—}23_P216004), OR6T1 (A_{—}23_P161815), TFDP3 (A_{—}23_P10513), (A_{—}23_P159693), PRSS21 (A_{—}23_P129602), RTP1 (A_{—}23_P155407), COX411 (A_{—}23_P141028), RPS6KA2 (A_{—}23_P335920), GGA2 (A_{—}23_P163732), C14orf58 (A_{—}23_P140364), IFNAR2 (A_{—}23_P211083), NPDO14 (A_{—}23_P34396), FLJ10154 (A_{—}23_P162776), ATP9A (A_{—}23_P102664), NKX2-8 (A_{—}23_P341547), OR8U1 (A_{—}23_P13244), KIAA1128 (A_{—}23_P404108), CCR1 (A_{—}23_P6849), FLJ10916 (A_{—}23_P259201), BAGE (A_{—}23_P388331), CDC6 (A_{—}23_P49972), THOC4 (A_{—}23_P152984), (A_{—}23_P104970), NCKIPSD (A_{—}23_P29634), MYOC (A_{—}23_P23783), (A_{—}23_P157835), (A_{—}23_P46649), PARP1 (A_{—}23_P114783), ZNF278 (A_{—}23_P211459), (A_{—}23_P51082), UNQ3045 (A_{—}23_P122600), RPL4 (A_{—}23_P58031), MYBL2 (A_{—}23_P143184), TM4SF6 (A_{—}23_P171143), KCNC3 (A_{—}23_P101516), C21orf105 (A_{—}23_P154915), LOC220070 (A_{—}23_P388932), DUSP22 (A_{—}23_P120252), MGC16385 (A_{—}23_P343843), APG10L (A_{—}23_P92824), ITSN2 (A_{—}23_P28201), MGC14816 (A_{—}23_P330581), UGT2B28 (A_{—}23_P212968), C13orf7 (A_{—}23_P25638), PPARGC1B (A_{—}23_P213959), FLJ14712 (A_{—}23_P381505), PCQAP (A_{—}23_P154954), SLC39A7 (A_{—}23_P70571), SPFH1 (A_{—}23_P202029), ITGAL (A_{—}23_P206806), RBX1 (A_{—}23_P211550), CART1 (A_{—}23_P95200), SURB7 (A_{—}23_P2262), MAP7 (A_{—}23_P122736), MGC20533 (A_{—}23_P141965), WDR10 (A_{—}23_P212440), GNL3L (A_{—}23_P22499), (A_{—}23_P426270), CCKBR (A_{—}23_P162007), RIT2 (A_{—}23_P170050), LOC144438 (A_{—}23_P98960), C14orf87 (A_{—}23_P65370), PLD2 (A_{—}23_P4308), CRYM (A_{—}23_P77731), (A_{—}23_P16408), DKFZp7621137 (A_{—}23_P157022), FGF14 (A_{—}23_P88033), (A_{—}23_P256260), GRIK4 (A_{—}23_P116249), CLDN5 (A_{—}23_P6321), KLHL10 (A_{—}23_P27128), COL3A1 (A_{—}23_P142527), RABGAP1L (A_{—}23_P200325), C1orf27 (A_{—}23_P282), KCNK3 (A_{—}23_P91104), ZBED1 (A_{—}23_P217508), (A_{—}23_P90732), Sep-06 (A_{—}23_P22613), GCM2 (A_{—}23_P59285), TH (A_{—}23_P258633), (A_{—}23_P46068), CPN1 (A_{—}23_P98147), VAMP1 (A_{—}23_P105545), NUPL2 (A_{—}23_P123039), NDUFC2 (A_{—}23_P2152), HYAL1 (A_{—}23_P69329), MGC57211 (A_{—}23_P420-431), QKI (A_{—}23_P81759), LOC51204 (A_{—}23_P61738), FLJ90650 (A_{—}23_P58557), EFNA5 (A_{—}23_P167497), VN1R4 (A_{—}23_P78656), TCF2 (A_{—}23_P207557), MRE11A (A_{—}23_P150189), (A_{—}23_P156811), FIBCD1 (A_{—}23_P112187), ZNRF1 (A_{—}23_P163858), GK00 (A_{—}23_P49616), CDADC1 (A_{—}23_P48299), FBXO15 (A_{—}23_P342709), FLJ21019 (A_{—}23_P152755), POLR3B (A_{—}23_P87730), (A_{—}23_P36205), (A_{—}23_P200699), ALLC (A_{—}23_P108734), ADCY8 (A_{—}23_P169989), TMEM41A (A_{—}23_P80831), KIAA1909 (A_{—}23_P81640), PTPN9 (A_{—}23_P124485), CAGLP (A_{—}23_P62588), RNF126 (A_{—}23_P314086), PLEKHG3 (A_{—}23_P76901), C10orf72 (A_{—}23_P304509), CXorf53 (A_{—}23_P217659), FLJ12892 (A_{—}23_P384056), (A_{—}23_P171007), EPN2 (A_{—}23_P89310), KLF11 (A_{—}23_P319512), PLA2G4B (A_{—}23_P403-424), KCNN1 (A_{—}23_P119578), DYRK1A (A_{—}23_P211126), R-spondin (A_{—}23_P22013), CACNG7 (A_{—}23_P4782), SLC22A8 (A_{—}23_P87219), HLA-DMA (A_{—}23_P42304), DEF6 (A_{—}23_P321913), C20orf36 (A_{—}23_P210827), MCM6 (A_{—}23_P90612), OVGP1 (A_{—}23_P103756), (A_{—}23_P217727), MARVELD3 (A_{—}23_P152428), (A_{—}23_P72434), FRMD3 (A_{—}23_P135123), KCND1 (A_{—}23_P217218), LOC134548 (A_{—}23_P400-406), (A_{—}23_P206598), MGC42090 (A_{—}23_P416821), GBF1 (A_{—}23_P161237), ROS1 (A_{—}23_P70278), PTPRH (A_{—}23_P101642), MYLK (A_{—}23_P132428), LOC93349 (A_{—}23_P337753), FMNL1 (A_{—}23_P89365), PIN1L (A_{—}23_P267), ZNF307 (A_{—}23_P133868), MGC4659 (A_{—}23_P61987), ATP11A (A_{—}23_P105908), ILF3 (A_{—}23_P435987), CCBE1 (A_{—}23_P55544), (A_{—}23_P136724), PHF12 (A_{—}23_P305761), ALOX15B (A_{—}23_P60627), WWOX (A_{—}23_P206413), CNOT1 (A_{—}23_P77371), ERBB2 (A_{—}23_P89249), C2GNT3 (A_{—}23_P122077), E2F2 (A_{—}23_P125990), DKFZP566N034 (A_{—}23_P39550), TGM1 (A_{—}23_P65617), SMAP-1 (A_{—}23_P218170), TNK2 (A_{—}23_P324931), FLJ13291 (A_{—}23_P3562), PTK6 (A_{—}23_P56978), PGM2L1 (A_{—}23_P396765), (A_{—}23_P250528), KRT6A (A_{—}23_P87653), LOC93109 (A_{—}23_P428840), SENP3 (A_{—}23_P163997), STK22B (A_{—}23_P40516), PDCD11 (A_{—}23_P161257), ABCA2 (A_{—}23_P43504), (A_{—}23_P155582), MYOM1 (A_{—}23_P96271), GDDR (A_{—}23_P61318), CROT (A_{—}23_P168669), PLEKHA1 (A_{—}23_P115792), (A_{—}23_P26674), MGC20410 (A_{—}23_P370682), CHRM3 (A_{—}23_P401-472), CHRNA10 (A_{—}23_P411188), LGR6 (A_{—}23_P23837), KIAA0514 (A_{—}23_P98115), ABCG4 (A_{—}23_P203231), (A_{—}23_P38978), KIF4A (A_{—}23_P148475), CRYBB1 (A_{—}23_P143621), SELE (A_{—}23_P97112), LOC199675 (A_{—}23_P330561), APOC4 (A_{—}23_P27450), AGTRL1 (A_{—}23_P318860), BACH2 (A_{—}23_P30633), OR8A1 (A_{—}23_P75677), RNF13 (A_{—}23_P29378), TBX21 (A_{—}23_P141555), VCX2 (A_{—}23_P171188), TP531NP1 (A_{—}23_P168882), BIRC8 (A_{—}23_P90058), SRISNF2L (A_{—}23_P18202), MGC11349 (A_{—}23_P211829), MTM1 (A_{—}23_P62128), NAP1L2 (A_{—}23_P125705), IGSF4C (A_{—}23_P5070), (A_{—}23_P214511), GNB4 (A_{—}23_P18186), FN3KRP (A_{—}23_P77813), CD1A (A_{—}23_P402670), IPO13 (A_{—}23_P384600), (A_{—}23_P44054), SLC37A4 (A_{—}23_P35970), ATR (A_{—}23_P136054), GNAZ (A_{—}23_P416581), ZFPM2 (A_{—}23_P168909), PAPPA (A_{—}23_P351270), BVES (A_{—}23_P502782), MMP15 (A_{—}23_P100177), SFXN2 (A_{—}23_P161253), AQP6 (A_{—}23_P204712), OR6S1 (A_{—}23_P54075), C10orf94 (A_{—}23_P115805), SQSTM1 (A_{—}23_P81401), SEC3L1 (A_{—}23_P113972), (A_{—}23_P104867), OR52J3 (A_{—}23_P53119), POLD2 (A_{—}23_P71140), KIAA0363 (A_{—}23_P93937), INSL5 (A_{—}23_P51479), ZNF76 (A_{—}23_P8133), SOX18 (A_{—}23_P255418), C14orf27 (A_{—}23_P65388), (A_{—}23_P10091), BAPX1 (A_{—}23_P386254), ADRB2 (A_{—}23_P145024), TRIM49 (A_{—}23_P1575), ODZ1 (A_{—}23_P257263), BTD (A_{—}23_P155348), UMODL1 (A_{—}23_P315964), C9orf19 (A_{—}23_P414913), FLJ23834 (A_{—}23_P348253), TNRC5 (A_{—}23_P19352), (A_{—}23_P33429), DKFZP434H2010 (A_{—}23_P23617), LOC220929 (A_{—}23_P161156), PIGC (A_{—}23_P86250), (A_{—}23_P99498), DTNA (A_{—}23_P208158), GPR161 (A_{—}23_P354320), GNRH1 (A_{—}23_P254594), KRTAP3-3 (A_{—}23_P89649), (A_{—}23_P130496), RIP (A_{—}23_P66758),
wherein an increase in the level of expression of at least one gene selected from group (a) and/or (b) and/or (c) and/or (d) and/or wherein a decrease in the level of expression of at least one gene selected from group (e) and/or (f) and/or (g) and/or (h) indicates a reduced level or activity of HDAC2 and is therefore indicative of cancer.

6. The method of claim 5 wherein the panel of genes comprises at least one gene taken from those genes listed in groups (a) and/or (b) and/or (e) and/or (f).

7. The method of claim 4 wherein an increase in the expression of any one or more of NCOA4, CTSB, TBCD, PPP2R4 and/or CORO1C indicates a reduced level or activity of HDAC2 and is therefore indicative of cancer.

8. The method of claim 1 wherein analysis of HDAC2 recruitment to one or more gene promoters is utilised in order to determine the level or activity of HDAC2.

9. The method of claim 8 wherein a loss of recruitment of HDAC2 at one or more gene promoters indicates a reduced level or activity of HDAC2 and is therefore indicative of cancer.

10. The method of claim 9 wherein the one or more gene promoters comprise the HDAC2 and/or TBCD and/or PPP2R4 and/or CORO1C promoter.

11. The method of claim 1 wherein analysis of histone acetylation is carried out in order to determine the level or activity of HDAC2.

12. The method of claim 11 wherein an increase in the acetylation levels of one or more histones indicates a reduced level or activity of HDAC2 and is therefore indicative of cancer.

13. The method of claim 12 wherein the one or more histones comprises histone H3 and/or histone H4.

14. The method of claim 1 wherein the level or activity of HDAC2 is compared to a control sample in which HDAC2 is known to be expressed and/or active and/or a control sample in which HDAC2 is known to be inactive and/or not expressed.

15. A method for predicting the probability of successful treatment of cancer with a hydroxamic acid based HDAC inhibitor comprising, in a sample obtained from a subject, determining the level or activity of HDAC2, wherein a reduced level or activity of HDAC2 is indicative of a low probability of successful treatment with the hydroxamic acid based HDAC inhibitor.

16. A method of selecting a suitable treatment regimen for cancer comprising, in a sample obtained from a subject, determining the level or activity of HDAC2, wherein a reduced level or activity of HDAC2 indicates that treatment using a hydroxamic acid based HDAC inhibitor is unsuitable.

17. The method of claim 16, wherein a reduced level or activity of HDAC2 leads to treatment using a carboxylic acid based HDAC inhibitor being selected.

18. A method of selecting a suitable treatment regimen for cancer comprising, in a sample obtained from a subject, determining the level or activity of HDAC2, wherein a reduced level or activity of HDAC2 indicates that treatment using a carboxylic acid based HDAC inhibitor should be selected.

19. (canceled)

20. A method for treating cancer in a subject using a carboxylic acid based HDAC inhibitor, the method comprising selecting a subject for treatment according to the method of claim 1.

21. A method for treating cancer in a subject, said subject having a reduced level or activity of HDAC2, comprising reconstitution of HDAC2 activity in the subject.

22. The method of claim 21 wherein reconstituting HDAC2 activity comprises delivery of wild type copies of the HDAC2 gene into the subject.

23. The method of claim 22 wherein delivery comprises use of a vector.

24. The method of claim 23 wherein the vector comprises an adenovirus.

25. The method of claim 1 wherein the cancer is associated with microsatellite instability.

26. The method of claim 1 wherein the cancer comprises any of colorectal, gastric or endometrial cancer.

27. The method of claim 26 wherein the colorectal cancer comprises hereditary nonpolyposis colon cancer or sporadic colorectal cancer.
